(19) United States
(12) Patent Application Publication
(10) Pub. No.: US 2004/0181075 A1
(43) Pub. Date: Sep. 16, 2004

(54) Process of making chalcone derivatives

(76) Inventors: M. David Weingarten, Cummings, GA (US); Liming Ni, Duluth, GA (US); Charles Q. Meng, Alpharetta, GA (US); Kimberly J. Worsencroft, Alpharetta, GA (US); James A. Sikorski, Atlanta, GA (US)

Correspondence Address: KING & SPALDING LLP, 191 PEACHTREE STREET, N.E., ATLANTA, GA, 30303-1763, US

(21) Appl. No.: 10/741,283

(22) Filed: Dec. 19, 2003

Related U.S. Application Data
(60) Provisional application No. 60/435,611, filed on Dec. 19, 2002.

(51) Int. Cl. C07D 257/02; C07D 207/46

(52) U.S. Cl. 548/253; 548/577

(57) ABSTRACT

This invention is a novel methods of manufacturing chalcones that includes reacting a carbon-linked heteroaryl or heterocyclic substituted benzaldehyde with an acetophenone in a solvent or mixture of solvents in the presence of LiOMe. Also provided are new chalcones for the treatment of medical conditions.

This application claims priority to U.S. Provisional Patent Application Serial No. 60/435,611, filed Dec. 19, 2002.

### FIELD

This application is in the area of processes for the manufacture of chalcones.

### BACKGROUND OF THE INVENTION

Chalcone (1,3-bis-aromatic-prop-2-en-1-ones) compounds are natural products related to flavonoids. U.S. Pat. No. 6,608,101 filed Jun. 20, 2001 and U.S. patent application Ser. No. 10/324,987, filed Dec. 19, 2002, disclose chalcone compounds useful as VCAM-1 inhibitors and suitable for the treatment of medical disorders, including inflammatory and cardiovascular diseases. The specifications of these patent applications disclose numerous compounds and methods of manufacturing such compounds.

PCT WO 99/00114 (PCT/DK98/00283) discloses the use of certain chalcones, 1,3-bis-aromatic-propan-1-ones (dihydrochalcones), and 1,3-bisaromatic-prop-2-yn-1-ones for the preparation of pharmaceutical compositions for the treatment of prophylaxis of a number of serious diseases including i) conditions relating to harmful effects of inflammatory cytokines, ii) conditions involving infection by Helicobacter species, iii) conditions involving infections by viruses, iv) neoplastic disorders, and v) conditions caused by microorganisms or parasites.

U.S. Pat. No. 4,085,135 discloses 2′-(carboxymethoxy)-chalcones with antigastric and antiduodenal ulcer activities.

Japanese Patent No. 04217621 to Tomomi discloses siloxane chalcone derivatives in sunscreens.

U.S. Pat. No. 4,085,135 to Kyogoku et al. discloses a process for preparation of 2′-(carboxymethoxy)-chalcones having antigastric and anti duodenal activities with low toxicity and high absorptive ratio in the body.

U.S. Pat. No. 4,855,438 discloses a process for preparing optically active 2-hydroxyethylazole derivatives which have fungicidal and plant growth-regulating action by reacting an α-β-unsaturated ketone which could include a chalcone or a chalcone derivative with an enantiomerically pure oxathiolane in the presence of a strongly basic organometallic compound and at temperatures ranging from −80 to 120° C.

European Patent No 307762 assigned to Hofmann-La Roche discloses substituted phenyl chalcones.

E. Bakhite et al. in J. Chem. Tech. Biotech. 1992, 55, 157-161, disclosed a process for the preparation of some phenyloxazole derivatives of chalcone by condensing 5-(p-acetylphenyl)-2-phenyloxazole with aromatic aldehydes.

Herencia, et al., in Synthesis and Anti-inflammatory Activity of Chalcone Derivatives, Bioorganic & Medicinal Chemistry Letters 8 (1998) 1169-1174, discloses certain chalcone derivatives with anti-inflammatory activity.

Hsieh, et al., Synthesis and Antiinflammatory Effect of Chalcones, J. Pharm. Pharmacol. 2000, 52; 163-171 describes that certain chalcones have potent antiinflammatory activity.

Zwaagstra, et al., Synthesis and Structure-Activity Relationships of Carboxylated Chalcones: A Novel Series of CysLT₁ (LT₄) Receptor Antagonists; J. Med. Chem., 1997, 40, 1075-1089 discloses that in a series of 2-, 3-, and 4-(2-quinolinylmethoxy)- and 3- and 4-[2-(2-quinolinyl)ethenyl]-substituted, 2′, 3′, 4′, or 5′ carboxylated chalcones, certain compounds are CysLT₁ receptor antagonists.

JP 63010720 to Nippon Kayaku Co., LTD discloses that certain chalcone derivatives can be used in treating allergies.

JP 06116206 to Morinaga Milk Industry Co. Ltd, Japan, discloses certain substituted chalcones.

U.S. Pat. No. 6,046,212 to Kowa Co. Ltd. discloses heterocyclic ring-containing chalcones as antiallergic agents.

Chalcones have been reviewed by Dimmock, et al., in Bioactivities of Chalcones, Current Medicinal Chemistry 1999, 6, 1125-1149; Liu. et al., Antimalarial Alkoxylated and Hydroxylated Chalones: Structure-Activity Relationship Analysis, J. Med. Chem. 2001, 44, 4443-4452; Herencia et al, Novel Anit-inflammatory Chalcone Derivatives Inhibit the Induction of Nitric Oxide Synthase and Cyclooxygenase-2 in Mouse Peritoneal Macrophages, FEBS Letters, 1999, 453, 129-134; and Hsieh et al., Synthesis and Anti-inflammatory Effect of Chalcones and Related Compounds, Pharmaceutical Research, 1998, Vol.15, No. 1, 39-46.

Given the large number of chalcones with medical properties, there is needed a method of manufacturing chalcone derivatives that is efficient and provides sufficient yields.

Therefore, it is an object of the present invention to provide methods for the manufacture of chalcones.

It is another object to provide chalcone derivatives that are suitable as therapeutics.

### SUMMARY OF THE INVENTION

A process of manufacturing a chalcone that includes reacting a carbon-linked heteroaryl or heterocyclic substituted benzaldehyde with an acetophenone in a solvent or mixture of solvents in the presence of LiOMe. In a particular embodiment, the invention provides methods of manufacturing a compound of Formula I or salts thereof

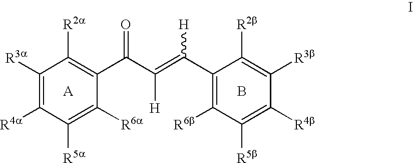

wherein

R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR, wherein y is 1, 2, 3, 4, 5, or 6, C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;

wherein at least one of R^{2α}, R^{3α}, R^{4α}, R^{5α}, and R^{6α} may be selected from the group consisting of cyano, tetrazol-5-yl, C(O)OH, C(O)OR², (CH₂)_{y}C(O)OR¹ wherein y is 1, 2, 3, 4, 5, or 6, C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR, —SO₂NHC(O)NHR², —SO₂NHC(O)N(R²)₂, and —SO₂NHC(O)NR⁷R⁸;

R^{2β}, R^{3β}, R^{4β}, R^{5β}, and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, lower alkyl S(O)-lower alkyl, lower alkyl-S(O)₂-lower alkyl, arylsulfinyl lower alkyl, arylsulfonyl lower alkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alditol, carbohydrate, polyol alkyl, alkoxy, lower alkoxy, alkoxy alkoxy alkoxy, —(O(CH₂)₂)₁₋₃—O-lower alkyl, polyoxyalkylene, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂—NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂N₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR¹, —C(O)NHSO₂N(R²), —C(O)NHSO₂NR⁷R⁸, —C(CH₃)₂C(O)OH, and —(CH₂)_{y}C(O)OH, wherein y is 1, 2, 3, 4, 5, or 6, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl; comprising reacting a carbon-linked heteroaryl or heterocyclic substituted benzaldehyde of Formula II

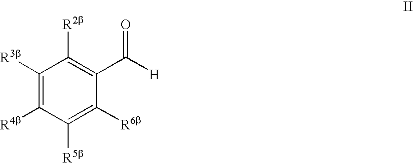

with an acetophenone of Formula III

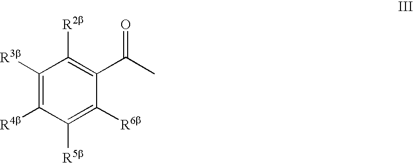

wherein R^{2α}, R^{3α}, R^{4α}, R^{5α}, R^{6α}, R^{2β}, R^{3β}, R^{4β}, R^{5β}, and R^{6β} for Formulas II and III are as defined above;

in a solvent or mixture of solvents in the presence of LiOMe.

Also included in the invention are specific compounds, pharmaceutical compositions and methods of using such compounds and pharmaceutical compositions to treat diseases.

### DETAILED DESCRIPTION OF THE INVENTION

The invention encompasses methods of manufacturing compounds of Formula I

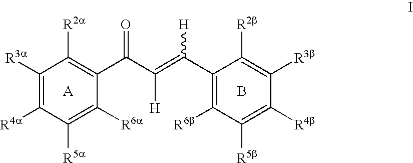

wherein

R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR¹ wherein y is 1, 2, 3, 4, 5, or 6, C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², —C(O)NHSO₂R², th iol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;

wherein at least one of R^{2α}, R^{3α}, R^{4α}, R^{5α}, and R^{6α} must be selected from the group consisting of cyano, tetrazol-5-yl, C(O)OH, C(O)OR², (CH₂)_{y}C(O)OR¹ wherein y is 1, 2, 3, 4, 5, or 6, C(R¹)₂C(O)OR¹, —C(O)NH₂, —C(O)NHR², —C(O)N(R²)₂, —C(O)NR⁷R⁸, C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂N⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂—SO₂NHC(O)N(R²) 2, and —SO₂NHC(O)NR⁷R⁸;

R^{2β}, R^{3β}, R^{4β}, R^{5β}, and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, lower alkyl S(O)-lower alkyl, lower alkyl-S(O)₂-lower alkyl, arylsulfinyl lower alkyl, arylsulfonyl lower alkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alditol, carbohydrate, polyol alkyl, alkoxy, lower alkoxy, alkoxy alkoxy alkoxy, —(O(CH₂)₂)₁₋₃—O-lower alkyl, polyoxyalkylene, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R²)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NRC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, C(O)NHSO₂R², —C(O)NHSO₂NHR², —C(O)NHSO₂N(R²), —C(O)NHSO₂NR⁷R⁸, —C(CH₃)₂C(O)OH, and —(CH₂)_{y}C(O)OH, wherein y is 1, 2, 3, 4, 5, or 6, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

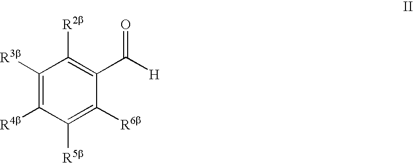

with an acetophenone of Formula III

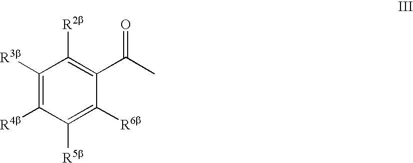

wherein R^{2α}, R^{3α}, R^{4α}, R^{5α}, R^{6α}, R^{2β}, R^{3β}, R^{4β}, R^{5β}, and R^{6β} for Formulas II and III are as defined above;

in a solvent or mixture of solvents in the presence of LiOMe.

The following embodiments of the invention are intended to illustrate the invention and are not intended to limit the invention in any way.

A 1^{st} embodiment of the invention is a method of manufacturing a compound of Formula I or salts therof

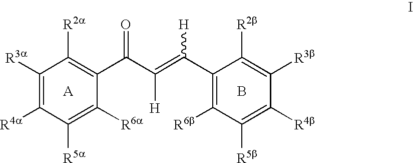

wherein

R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR¹ wherein y is 1, 2, 3, 4, 5, or 6, —C(R¹)₂C(O)OR¹, —C(O)NH₂, —C(O)NHR², —C(O)N(R²)₂, —C(O)NR⁷R⁸, —C(O)NHC(O)NHR¹, —C(O)NHC(O)N(R²)₂, —C(O)NHC(O)NR⁷R⁸, —C(O)NRSO₂NHR², —C(O)NHSO₂N(R²)₂, —C(O)NHSO₂NR⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;

wherein at least one of R^{2α}, R^{3α}, R^{4α}, R^{5α}, and R^{6α} must be selected from the group consisting of cyano, tetrazol-5-yl, C(O)OH, C(O)OR², (CH₂)_{y}C(O)OR¹ wherein y is 1, 2, 3, 4, 5, or 6, —C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, and —SO₂NHC(O)NR⁷R⁸;

R^{2β}, R^{3β}, R^{4β}, R^{5β}, and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, lower alkyl S(O)-lower alkyl, lower alkyl-S(O)₂-lower alkyl, arylsulfinyl lower alkyl, arylsulfonyl lower alkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alditol, carbohydrate, polyol alkyl, alkoxy, lower alkoxy, alkoxy alkoxy alkoxy, —(O(CH₂)₂)₁₋₃-O-lower alkyl, polyoxyalkylene, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR²², —NHSO₂R², —NHSO₂NR⁷⁸R², —N(C(O)NHR²)₂, —NR² SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, and —(CH₂)_{y}C(O)OH, wherein y is 1, 2, 3, 4, 5, or 6, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

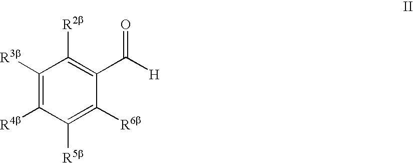

with an acetophenone of Formula III

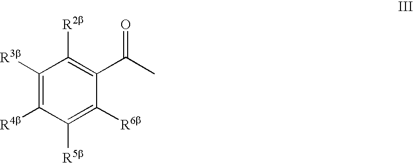

wherein R^{2α}, R^{3α}, R^{4α}, R^{5α}, R^{6α}, R^{2β}R^{3β}, R^{4β}, R^{5β}, and R^{6β} for Formulas II and III are as defined above;

in a solvent or mixture of solvents in the presence of LiOMe.

A 2^{nd} embodiment of the invention is a method of manufacturing a compound of Formula I or salts thereof

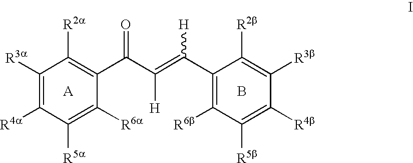

wherein

R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR¹ wherein y is 1, 2, 3, 4, 5, or 6, —C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²), —C(O)NHSO₂NR⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸;

wherein at least one of R^{2α}, R^{3α}, R^{4α}, R^{5α}, and R^{6α} must be selected from the group consisting of cyano, tetrazol-5-yl, C(O)OH, C(O)OR², (CH₂)_{y}C(O)OR, wherein y is 1, 2, 3, 4, 5, or 6, —C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

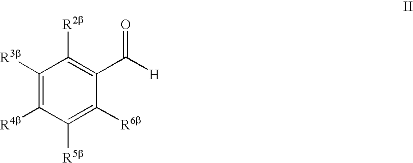

with an acetophenone of Formula III

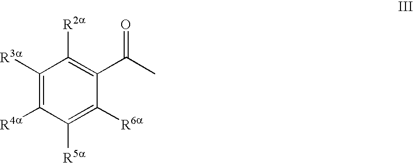

wherein R^{2α}, R^{3α}, R^{4α}, R^{5α}R⁶, R^{2β}, R^{3β}, R^{4β}, R^{5β}, and R^{6β} for Formula II and III are as defined above;

in a solvent or mixture of solvents in the presence of LiOMe.

A 3^{rd} embodiment of the invention is a method of manufacturing a compound of Formula I or salts thereof

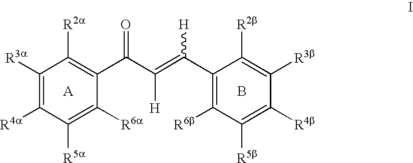

wherein

R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR¹ wherein y is 1, 2, 3, 4, 5, or 6, C(R¹)₂C(O)OR¹;

wherein at least one of R^{2α}, R^{3α}, R^{4α}, R^{5α}, and R^{6α} must be selected from the group consisting of cyano, tetrazol-5-yl, C(O)OH, C(O)OR², (CH₂)_{y}C(O)OR¹ wherein y is 1, 2, 3, 4, 5, or 6, —C(R¹)₂C(O)OR¹;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl; comprising: reacting a carbon-linked heteroaryl or heterocyclic substituted benzaldehyde of Formula II

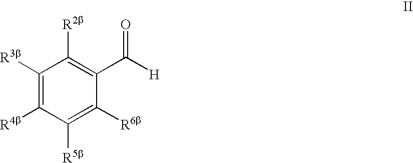

with an acetophenone of Formula III

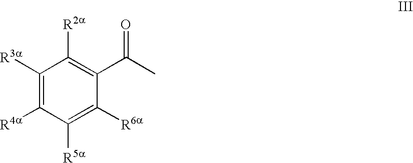

wherein R^{2α}R³, R⁴, R^{5α}, R^{6α} R^{2β}R^{3β}, R^{4β}, R^{5β}, and R^{6β} for Formula II and III are as defined above; in a solvent or mixture of solvents in the presence of LiOMe.

A 4^{th} embodiment of the invention is a method of manufacturing a compound of Formula I or salts thereof

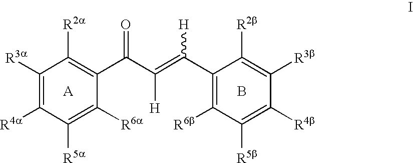

wherein

R^{2α}, R^{3α}, R^{4α}, R^{5α}, and R^{6α} are independently selected from the group consisting of hydrogen, C(O)OH, C(O)OR², (CH₂)_{y}C(O)OR¹ wherein y is 1, 2, 3, 4, 5, or 6, —C(R¹)₂C(O)OR¹;

wherein at least one of R^{2α}, R^{3α}, R^{4α}, R^{5α}, and Re must be selected from the group consisting of C(O)OH, C(O)OR², (CH₂)_{y}C(O)OR¹ wherein y is 1, 2, 3, 4, 5, or 6, —C(R¹)₂C(O)OR¹;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, —C(O)NR⁷R⁸, and —C(O)N(R²)₂;

R² is independently selected from the group consisting of alkyl, lower alkyl, cycloalkyl, aryl, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, —C(O)NR⁷R⁸, —C(O)NH₂, and —C(O)N(R²)₂;

R⁷ and R⁸ are independently selected from the group consisting of alkyl, alkenyl and aryl and linked together forming a 5- to 7-membered monocyclic, bicylic, tricyclic or benzofused ring;

wherein R⁷ and R⁸ can be optionally substituted with one or more selected from the group consisting of alkyl, lower alkyl, cycloalkyl, acyl, hydroxy, hydroxyalkyl, heterocyclic, amino, aminoalkyl, —NR⁷R⁸, alkoxy, oxo, cyano, —C(O)NR⁷ R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl; comprising:

reacting a carbon-linked heteroaryl or heterocyclic substituted benzaldehyde of Formula II

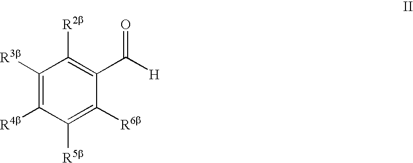

with an acetophenone of Formula III

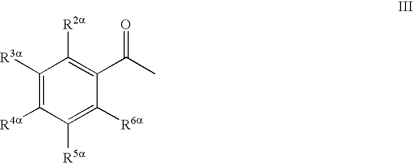

wherein R^{2α}, R^{3α}, R^{4α}, R^{5α}, R^{6α}, R^{2β}, R^{3β}, R^{4β}, R^{5β}, and R^{6β} for Formula II and III are as defined above;

in a solvent or mixture of solvents in the presence of LiOMe.

A 5^{th} embodiment of the invention is a method of manufacturing a compound of Formula I or salts thereof

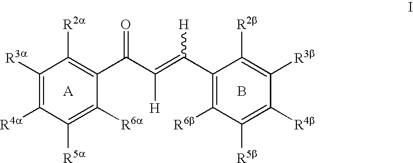

wherein

R^{2α}, R^{3α}, R^{4α}, R^{5α}, and R^{6α} are independently selected from the group consisting of hydrogen, C(O)OH, and C(O)OR²;

wherein at least one of R^{2α}, R^{3α}, R^{4α}, R^{5α}, and R^{6α} must be selected from the group consisting of C(O)OH and C(O)OR²;

R^{2β}, R^{3β}, R^{4β}, R^{5β}, and R^{6β} are independently selected from the group consisting of hydrogen, halo, alkoxy, alkoxy, heteroaryloxy, heterocyclic, and heteroaryl, all of which can be optionally substituted by one or more selected from the group consisting of halo, alkyl, lower alkyl, cycloalkyl, acyl, hydroxy, hydroxyalkyl, heteroaryl, heterocyclic, alkoxy, oxo, carboxy, carboxyalkyl, alkoxycarbonyl, and —C(O)N(R²)₂;

R² is independently selected from the group consisting of alkyl, lower alkyl, cycloalkyl, arylalkyl, and heteroarylalkyl, wherein all may be substituted by one or more selected from the group consisting of halo, alkyl, lower alkyl, cycloalkyl, acyl, hydroxy, heterocyclic, alkoxy, oxo, —C(O)NH₂, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

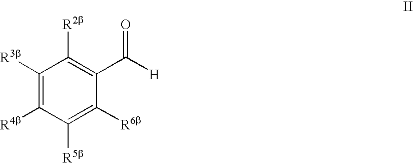

with an acetophenone of Formula III

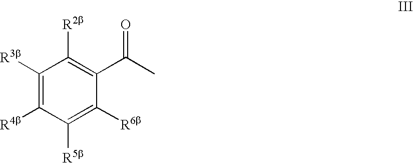

wherein R^{2α}, R^{3α}, R^{4α}, R^{5α}, R^{6α}, R^{2β}, R^{3β}, R^{4β}, R^{5β}, and R^{6β} for Formula II and III are as defined above; in a solvent or mixture of solvents in the presence of LiOMe.

A 6^{th} embodiment of the invention is a method of manufacturing a compound of Formula I or salts thereof

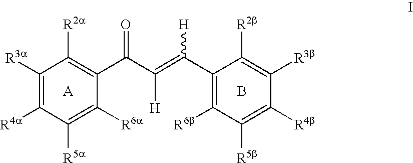

wherein

R^{2α}, R^{3α}, R^{4α}, R^{5α}, and R^{6α} are independently selected from the group consisting of hydrogen and C(O)OH;

wherein at least one of R^{2α}, R^{3α}, R^{4α}, R^{5α}, and R^{6α} must be C(O)OH;

R^{2β}, R^{3β}, R^{4β}, R^{5β}, and R^{6β} are independently selected from the group consisting of hydrogen, halo, alkoxy, alkoxy, heterocyclic, and heteroaryl, all of which can be optionally substituted by one or more selected from the group consisting of lower alkyl, hydroxy, hydroxyalkyl, heteroaryl, heterocyclic, alkoxy, alkoxycarbonyl, and —C(O)N(R²)₂;

R² is independently selected from the group consisting of alkyl, lower alkyl, arylalkyl, and heteroarylalkyl, wherein all may be substituted by one or more selected from the group consisting of lower alkyl, heterocyclic, alkoxy, —C(O)NH₂, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

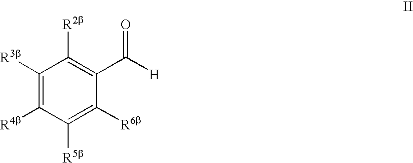

with an acetophenone of Formula III

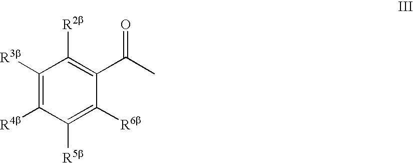

wherein R^{2α}, R^{3α}, R^{4α}, R^{5α}, R^{6α}, R^{2β}, R^{3β}, R^{4β}, R^{5β}, and R^{6β} for Formula II and III are as defined above;

in a solvent or mixture of solvents in the presence of LiOMe.

A 7^{th} embodiment of the invention is a method of manufacturing a compound selected from the group consisting of:

4-(3E-{4-Methoxy-2-[2-(2-methoxyethoxy)ethoxy]-5-thiophen-2-yl-phenyl}-acryloyl)-benzoic acid;

4-{3E-[4-(1-Carboxy-1-methyl-ethoxy)-2-methoxy-5-thiophen-2-yl-phenyl]-acryloyl}-benzoic acid;

4-[(2E)-3-(5-Benzo[b]thien-2-yl-2,4-dimethoxyphenyl)-1-oxo-2-propenyl]-benzoic acid;

4-[3E-(2,4-Dimethoxy-5-thiophen-2-yl-phenyl)-acryloyl]-benzoic acid;

4-[3E-(2,6-Dimethoxy-4-thiophen-2-yl-phenyl)-acryloyl]-benzoic acid;

4-{3E-[2,4-Dimethoxy-5-(5-methyl-thiophen-2-yl)-phenyl]-acryloyl}-benzoic acid;

4-[3E-(4-Methoxy-3-thiophen-2-yl-phenyl)-acryloyl]-benzoic acid;

4-[3E-(3-Thiophen-2-yl-phenyl)-acryloyl]-benzoic acid;

3-[3E-(2,4-Dimethoxy-5-thiophen-2-yl-phenyl)-acryloyl]-benzoic acid;

4-[3E-(3-Benzo[b]thiophen-2-yl-2,4-dimethoxy-phenyl)-acryloyl]-benzoic acid;

4-[3E-(2-Methoxy-5-thiophen-2-yl-phenyl)-acryloyl]-benzoic acid;

4-[3E-(2,4-Dimethoxy-5-pyrazin-2-yl-phenyl)-acryloyl]-benzoic acid;

4-(3E-{2-Methoxy-4-[2-(2-methoxy-ethoxy)-ethoxy]-5-thiophen-2-yl-phenyl}-acryloyl)-benzoic acid;

4-{3E-[4-(3-Hydroxy-2-hydroxymethyl-propoxy)-2-methoxy-5-thiophen-2-yl-phenyl]-acryloyl}-benzoic acid;

5-{5-[3-(4-Carboxy-phenyl)-3-oxo-E-propenyl]-2,4-dimethoxy-phenyl}-thiophene-2-carboxylic acid methyl ester;

4-[3E-(4-Ethoxy-2-methoxy-5-thiophen-2-yl-phenyl)-acryloyl]-benzoic acid;

4-[3E-(4-Hydroxy-2-methoxy-5-thiophen-2-yl-phenyl)-acryloyl]-benzoic acid;

4-[3E-(2,4-Dimethoxy-5-thiazol-2-yl-phenyl)-acryloyl]-benzoic acid;

2-{5-[3-(4-Carboxy-phenyl)-3-oxo-E-propenyl]-2,4-dimethoxy-phenyl}-pyrrole-1-carboxylic acid tert-butyl ester;

4-[3E-(2-Hydroxy-4-methoxy-5-thiophen-2-yl-phenyl)-acryloyl]-benzoic acid;

4-{3E-[2-(1-Carboxy-1-methyl-ethoxy)-4-methoxy-5-thiophen-2-yl-phenyl]-acryloyl}-benzoic acid;

4-{3E-[4-Methoxy-2-(2-morpholin-4-yl-ethoxy)-5-thiophen-2-yl-phenyl]-acryloyl}-benzoic acid, hydrochloride;

4-{3E-[5-(1H-Indol-2-yl)-2,4-dimethoxy-phenyl]-acryloyl}-benzoic acid;

4-{3E-[2-(3,5-Dimethyl-isoxazol-4-ylmethoxy)-4-methoxy-5-thiophen-2-yl-phenyl]-acryloyl}-benzoic acid;

4-[3E-(2-Pyrrolidin-1-yl-5-thiophen-2-yl-phenyl)-acryloyl]-benzoic acid;

4-{3E-[2-(3-Hydroxy-2-hydroxymethyl-propoxy)-4-methoxy-5-thiophen-2-yl-phenyl]-acryloyl}-benzoic acid;

4-{3E-[2-(3-Morpholin-4-yl-propoxy)-5-thiophen-2-yl-phenyl]-acryloyl}-benzoic acid, hydrochloride;

4-{3E-[4-Methoxy-2-(3-morpholin-4-yl-propoxy)-5-thiophen-2-yl-phenyl]-acryloyl}-benzoic acid, hydrochloride;

4-[3E-(2-Dimethylcarbamoylmethoxy-4-methoxy-5-thiophen-2-yl-phenyl)-acryloyl]-benzoic acid;

4-[3E-(4-Methoxy-2-{2-[2-(2-methoxy-ethoxy)-ethoxy]-ethoxy}-5-thiophen-2-yl-phenyl)-acryloyl]-benzoic acid;

4-{3E-[2,4-Dimethoxy-5-(2-methyl-thiazol-4-yl)-phenyl]-acryloyl}-benzoic acid;

4-{3E-[5-(1H-Benzoimidazol-2-yl)-2,4-dimethoxy-phenyl]-acryloyl}-benzoic acid;

4-[3E-(2-Carbamoylmethoxy-4-methoxy-5-thiophen-2-yl-phenyl)-acryloyl]-benzoic acid;

4-{3E-[4-Methoxy-2-(2-morpholin-4-yl-2-oxo-ethoxy)-5-thiophen-2-yl-phenyl]-acryloyl}-benzoic acid;

4-(3E-{4-Methoxy-2-[2-(1-methyl-pyrrolidin-2-yl)-ethoxy]-5-thiophen-2-yl-phenyl}-acryloyl)-benzoic acid, hydrochloride;

4-{3E-[2,4-Dimethoxy-5-(1H-pyrazol-4-yl)-phenyl]-acryloyl}-benzoic acid;

4-{3E-[2,4-Dimethoxy-5-(2H-tetrazol-5-yl)-phenyl]-acryloyl}-benzoic acid;

4-{3E-[5-(3H-Imidazo[4,5-b]pyridin-2-yl)-2,4-dimethoxy-phenyl]-acryloyl}-benzoic acid;

4-{3E-[2,4-Dimethoxy-5-(1-methyl-1H-indol-2-yl)-phenyl]-acryloyl}-benzoic acid;

4-[(2E)-3-(5-Benzofuran-2-yl-2,4-dimethoxyphenyl)-1-oxo-2-propenyl]-benzoic acid

4-{3E-[5-(2-Cyclopropyl-1H-imidazol-4-yl)-2,4-dimethoxy-phenyl]-acryloyl}-benzoic acid, hydrochloride; and

4-{3E-[5-(4-Isobutyl-4H-[1,2,4]triazol-3-yl)-2,4-dimethoxy-phenyl]-acryloyl}-benzoic acid;

comprising:

reacting a carbon-linked heteroaryl or heterocyclic substituted benzaldehyde of Formula II

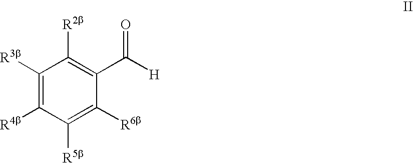

with an acetophenone of Formula III

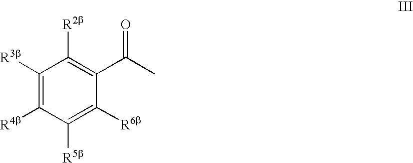

wherein

R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR¹ wherein y is 1, 2, 3, 4, 5, or 6, C(R¹)₂C(O)OR¹;

wherein at least one of R^{2α}, R^{3α}, R^{4α}, R^{5α}, and R^{6α}, must be selected from the group consisting of cyano, tetrazol-5-yl, C(O)OH, C(O)OR², (CH₂)_{y}C(O)OR¹ wherein y is 1, 2, 3, 4, 5, or 6, —C(R¹)₂C(O)OR¹;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl; in a solvent or mixture of solvents in the presence of LiOMe.

An 8^{th} embodiment of the invention is a method of manufacturing a compound of Formula I or salts thereof

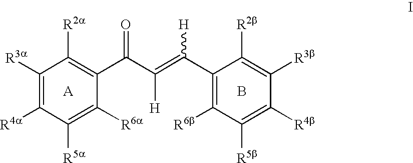

wherein

R^{2α}, R^{3α}, R^{4α}, R^{5α}, and R^{6α} are independently selected from the group consisting of hydrogen, thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR, —SO₂NHC(O)NHR², —SO₂NHC(O)N(R²)₂, —SO₂NHC(O)NR⁷R⁸;

wherein at least one of R^{2α}, R^{3α}, R^{4α}, R^{5α}, and R^{6α} must be selected from the group consisting of thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R², —SO₂NHC(O)R², —SR², —SO₂NHC(O)NHR², —SO₂NHC(O)N(R²)₂, and —SO₂NHC(O)NR⁷R⁸;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R², R³, R⁴, R⁵, and R^{6β} must be an optionally substituted carbon-carbon linked heterocyclic or heteroaryl; comprising:

reacting a carbon-linked heteroaryl or heterocyclic substituted benzaldehyde of Formula II

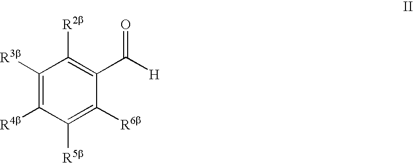

with an acetophenone of Formula III

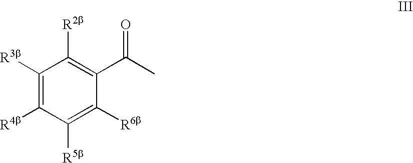

wherein R^{2α}, R^{3α}, R^{4α}, R^{5α}, R^{6α}, R^{2β}, R^{3β}, R^{4β}, R^{5β}, and R^{6β} for Formula II and III are as defined above;

in a solvent or mixture of solvents in the presence of LiOMe.

A 9^{th} embodiment of the invention is a method of manufacturing a compound of Formula I or salts thereof

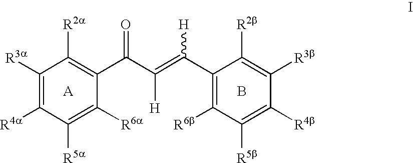

wherein

R^{2α}, R^{3α}, R^{4α}, R^{5α}, and R^{6α} are independently selected from the group consisting of hydrogen, —SO₂NH₂, —SO₂NHR², —SO₂N(R²)₂, SO₂NR⁷R⁸, —SO₂NHC(O)R², —SO₂NHC(O)NHR², —SO₂NHC(O)N(R²)₂, —SO₂NHC(O)NR⁷R⁸;

wherein at least one of R^{2α}, R^{3α}, R^{4α}, R^{5α}, and R^{6α} must be selected from the group consisting of —SO₂NH₂, —SO₂NHR², —SO₂N(R²)₂, SO₂NR⁷R⁸, —SO₂NHC(O)R², —SO₂NHC(O)NHR², —SO₂NHC(O)N(R²)₂, and —SO₂NHC(O)NR⁷R⁸;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, cycloalkyl, aryl, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, —C(O)NR⁷R⁸, —C(O)NH₂, and —C(O)N(R²)₂;

R⁷ and R⁸ are independently selected from the group consisting of alkyl, alkenyl and aryl and linked together forming a 5- to 7-membered monocyclic, bicylic, tricyclic or benzofused ring;

wherein R⁷ and R⁸ can be optionally substituted with one or more selected from the group consisting of alkyl, lower alkyl, cycloalkyl, acyl, hydroxy, hydroxyalkyl, heterocyclic, amino, aminoalkyl, —NR⁷R⁸, alkoxy, oxo, cyano, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

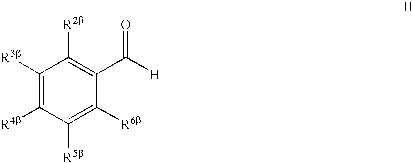

with an acetophenone of Formula III

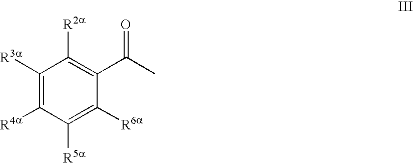

wherein R^{2α}R^{3α}, R^{4α}, R^{5α}, R^{6α}, R^{2β}, R^{3β}, R^{4β}, R^{5β}, and R^{6β} for Formula II and III are as defined above;

in a solvent or mixture of solvents in the presence of LiOMe.

A 10^{th} embodiment of the invention is a method of manufacturing a compound of Formula I or salts thereof

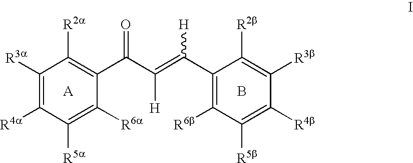

wherein

R^{2α}, R^{3α}, R^{4α}, R^{5α}, and R^{6α} are independently selected from the group consisting of hydrogen, —SO₂NH₂, —SO₂NHR², —SO₂N(R²)₂, and SO₂NR⁷R⁸;

wherein at least one of R^{2α}, R^{3α}, R^{4α}, R^{5α}, and R^{6α} must be selected from the group consisting of —SO₂NH₂, —SO₂NHR², —SO₂N(R²)₂, SO₂NR⁷R⁸, and —SO₂NHC(O)R²;

R², R^{3β}, R^{4β}, R^{5β}, and R^{6β} are independently selected from the group consisting of hydrogen, halo, alkoxy, heteroaryloxy, heterocyclic, and heteroaryl, all of which can be optionally substituted by one or more selected from the group consisting of halo, alkyl, lower alkyl, cycloalkyl, acyl, hydroxy, hydroxyalkyl, heteroaryl, heterocyclic, alkoxy, oxo, cyano, carboxy, carboxyalkyl, alkoxycarbonyl, and —C(O)N(R²)₂;

R² is independently selected from the group consisting of alkyl, lower alkyl, cycloalkyl, arylalkyl, and heteroarylalkyl wherein all may be substituted by one or more selected from the group consisting of halo, alkyl, lower alkyl, cycloalkyl, acyl, hydroxy, heterocyclic, alkoxy, oxo, —C(O)NH₂, and —C(O)N(R²)₂;

R⁷ and R⁸ are independently selected from the group consisting of alkyl, alkenyl and aryl and linked together forming a 5- to 7-membered monocyclic, bicylic, tricyclic or benzofused ring;

wherein R⁷ and R⁸ can be optionally substituted with one or more selected from the group consisting of lower alkyl, hydroxy, hydroxyalkyl, heterocyclic, amino, aminoalkyl, and cyano;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

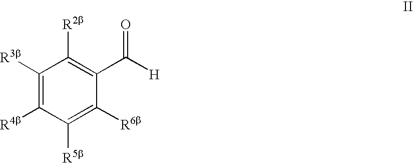

with an acetophenone of Formula III

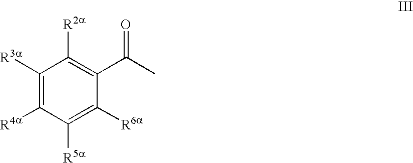

wherein R^{2α}, R^{3α}, R^{4α}, R^{5α}, R^{6α}, R^{2β}, R^{3β}, R^{4β}, R^{5β}, and R^{6β} for Formula II and III are as defined above; in a solvent or mixture of solvents in the presence of LiOMe.

An 11^{th} embodiment of the invention is a method of manufacturing a compound of Formula I or salts thereof

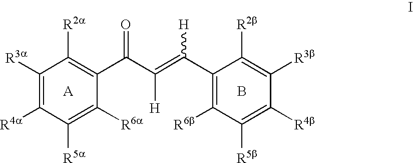

wherein

R^{2α}, R^{3α}, R^{4α}, R^{5α}, and R^{6α} are independently selected from the group consisting of hydrogen and —SO₂NH₂;

wherein at least one of R^{2α}, R^{3α}, R^{4α}, R^{5α}, and R^{6α} must be —SO₂NH₂;

R^{2β}, R^{3β}, R^{4β}, R^{5β}, and R^{6β} are independently selected from the group consisting of hydrogen, halo, alkoxy, heterocyclic, and heteroaryl, all of which can be optionally substituted by one or more selected from the group consisting of lower alkyl, hydroxy, hydroxyalkyl, heteroaryl, heterocyclic, alkoxy, cyano, alkoxycarbonyl, and —C(O)N(R²)₂;

R² is independently selected from the group consisting of alkyl, lower alkyl, arylalkyl, and heteroarylalkyl wherein all may be substituted by one or more selected from the group consisting of lower alkyl, heterocyclic, alkoxy, —C(O)NH₂, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

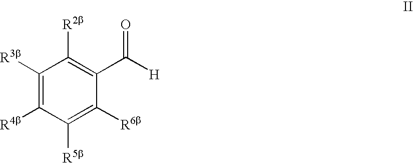

with an acetophenone of Formula III

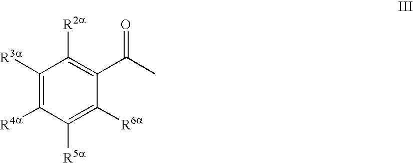

wherein R^{2α}, R^{3α}, R^{4α}, R^{5α}, R^{6α}, R^{2β}, R^{3β}, R^{4β}, R^{5β}, and R^{6β} for Formula II and III are as defined above;

in a solvent or mixture of solvents in the presence of LiOMe.

A 12^{th} embodiment of the invention is a method of manufacturing a compound selected from the group consisting of:

4-[3E-(5-Benzo[b]thiophen-2-yl-2,4-dimethoxy-phenyl)-acryloyl]-benzenesulfonamide;

4-{3E-[4-Methoxy-2-(2-morpholin-4-yl-ethoxy)-5-thiophen-2-yl-phenyl]-acryloyl}-benzenesulfonamide;

2-{5-Methoxy-2-[3-oxo-3-(4-aminosulfonyl-phenyl)-E-propenyl]-4-thiophen-2-yl-phenoxy}-2-methyl-propionic acid;

2-{2,4-Dimethoxy-5-[3-oxo-3-(4-aminosulfonyl-phenyl)-E-propenyl]-phenyl}-indole-1-carboxylic acid tert-butyl ester;

4-{3E-[5-(1H-Indol-2-yl)-2,4-dimethoxy-phenyl]-acryloyl}-benzenesulfonamide;

4-{3E-[4-Methoxy-2-(3-morpholin-4-yl-propoxy)-5-thiophen-2-yl-phenyl]-acryloyl}-benzenesulfonamide;

4-{3E-[2-(3-Hydroxy-2-hydroxymethyl-propoxy)-4-methoxy-5-thiophen-2-yl-phenyl]-acryloyl}-benzenesulfonamide;

4-{3E-[4-Methoxy-2-(1H-tetrazol-5-ylmethoxy)-5-thiophen-2-yl-phenyl]-acryloyl}-benzenesulfonamide;

4-{3E-[2,4-Dimethoxy-5-(1-methyl-1H-indol-2-yl)-phenyl]-acryloyl}-benzoic acid;

4-{3-[3E-(2,3-Dihydro-furan-2-yl)-phenyl]-acryloyl}-benzenesulfonamide;

4-{3E-[5-(2,5-Dihydro-furan-2-yl)-2,4-dimethoxy-phenyl]-acryloyl}-benzenesulfonamide;

4-{3E-[4-Methoxy-2-(6-methyl-pyridin-2-yloxy)-5-thiophen-2-yl-phenyl]-acryloyl}-benzenesulfonamide;

4-[3E-(2,4-Dimethoxy-5-pyridin-3-yl-phenyl)-acryloyl]-benzenesulfonamide;

4-{3E-[5-(2-Cyclopropyl-1H-imidazol-4-yl)-2,4-dimethoxy-phenyl]-acryloyl}-benzoic acid, hydrochloride;

4-{3E-[4-(3-Hydroxy-2-hydroxymethyl-propoxy)-2-methoxy-5-thiophen-2-yl-phenyl]-acryloyl}-benzenesulfonamide;

4-{3E-[2,4-Dimethoxy-5-(1-methyl-1H-indol-2-yl)-phenyl]-acryloyl}-benzenesulfonamide;

4-{3E-[5-(4-Isobutyl-4H-[1,2,4]triazol-3-yl)-2,4-dimethoxy-phenyl]-acryloyl}-benzenesulfonamide;

4-{3E-[5-(4-Isobutyl-4H-[1,2,4]triazol-3-yl)-2,4-dimethoxy-phenyl]-acryloyl}-benzoic acid;

4-{3E-[5-(2-Cyclopropyl-1H-imidazol-4-yl)-2,4-dimethoxy-phenyl]-acryloyl}-benzenesulfonamide;

4-{3E-[5-(3H-Imidazo[4,5-b]pyridin-2-yl)-2,4-dimethoxy-phenyl]-acryloyl}-benzenesulfonamide;

4-{3E-[2-(1H-Benzoimidazol-2-ylmethoxy)-4-methoxy-5-thiophen-2-yl-phenyl]-acryloyl}-benzenesulfonamide;

4-{3E-[4-Methoxy-2-(pyridin-2-ylmethoxy)-5-thiophen-2-yl-phenyl]-acryloyl}-benzenesulfonamide; and

4-{3E-[2-(Benzotriazol-1-ylmethoxy)-4-methoxy-5-thiophen-2-yl-phenyl]-acryloyl}-benzenesulfonamide;

comprising:

reacting a carbon-linked heteroaryl or heterocyclic substituted benzaldehyde of Formula II

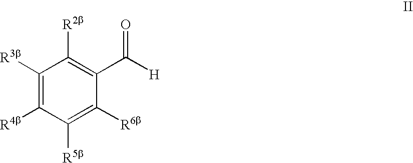

with an acetophenone of Formula III

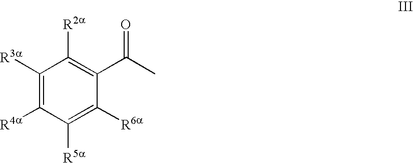

wherein

R^{2α}, R^{3α}, R^{4α}, R^{5α}, and R^{6α} are independently selected from the group consisting of hydrogen, thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸;

wherein at least one of R^{2α}, R^{3α}, R^{4α}, R^{5α}, and R^{6α}, must be selected from the group consisting of thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl; in a solvent or mixture of solvents in the presence of LiOMe.

A 13^{th} embodiment of the invention is a method of manufacturing a compound of Formula I or salts thereof

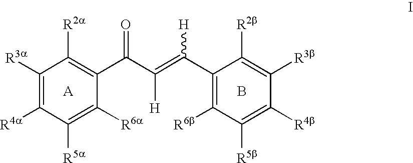

wherein

R^{2α}, R^{3α}, R^{4α}, R^{5α}, and R^{6α} are independently selected from the group consisting of hydrogen, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²), —C(O)NHSO₂NR⁷R⁸, —C(O)NHC(O)R², —C(O)NHSO₂R²;

wherein at least one of R^{2α}, R^{3α}, R^{4α}, R^{5α}, and R^{6α} must be selected from the group consisting of —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², —C(O)NHSO₂R²;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

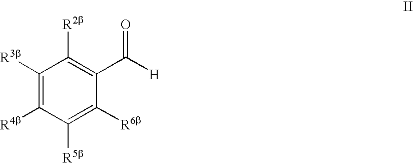

with an acetophenone of Formula III

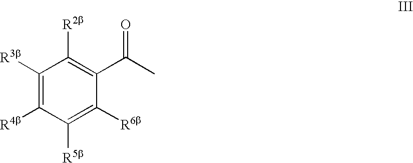

wherein R^{2α}, R^{3α}, R^{4α}, R^{5α}, R^{6α}, R^{2β}, R^{3β}, R^{4β}, R^{5β}, and R^{6β} for Formula II and III are as defined above; in a solvent or mixture of solvents in the presence of LiOMe.

A 14^{th} embodiment of the invention is a method of manufacturing a compound of Formula I or salts thereof

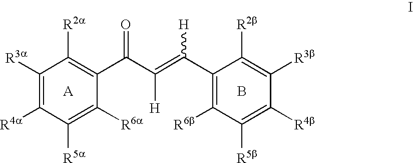

wherein

R^{2α}, R^{3α}, R^{4α}, R^{5α}, and R^{6α} are independently selected from the group consisting of hydrogen, —C(O)NH₂, —C(O)NHR², —C(O)N(R²)₂, —C(O)NR⁷R⁸, —C(O)NHSO₂NR⁷R⁸, —C(O)NHC(O)R², —C(O)NHSO₂R²;

wherein at least one of R^{2α}, R^{3α}, R^{4α}, R^{5α}, and R^{6α} must be selected from the group consisting of —C(O)NH₂, —C(O)NHR², —C(O)N(R²)₂, —C(O)NR⁷R⁸, —C(O)NHSO₂NR⁷R⁸, —C(O)NHC(O)R², —C(O)NHSO₂R²;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, cycloalkyl, aryl, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, —C(O)NR⁷R⁸, —C(O)NH₂, and —C(O)N(R²)₂;

R⁷ and R⁸ are independently selected from the group consisting of alkyl, alkenyl and aryl and linked together forming a 5- to 7-membered monocyclic benzofused ring;

wherein R⁷ and R⁸ can be optionally substituted with one or more selected from the group consisting of alkyl, lower alkyl, cycloalkyl, acyl, hydroxy, hydroxyalkyl, heterocyclic, amino, aminoalkyl, alkoxy, cyano, —C(O)NR⁷R⁸, and —C(O)N(R¹)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

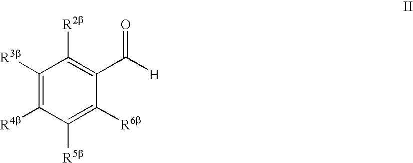

with an acetophenone of Formula III

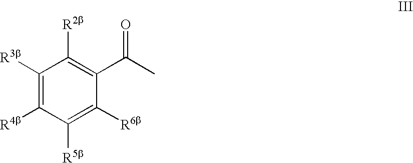

wherein R^{2α}, R^{3α}, R^{4α}, R^{5α}, R^{6α}, R^{2β}, R^{3β}, R^{4β}, R^{5β}, and R^{6β} for Formula II and III are as defined above; in a solvent or mixture of solvents in the presence of LiOMe.

A 15^{th} embodiment of the invention is a method of manufacturing a compound of Formula I or salts thereof

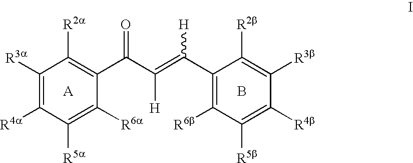

wherein

R^{2α}, R^{3α}, R^{4α}, R^{5α}, and R^{6α} are independently selected from the group consisting of hydrogen, —C(O)NH₂, —C(O)NHR², —C(O)NHC(O)R², —C(O)NHSO₂R²;

wherein at least one of R^{2α}, R^{3α}, R^{4α}, R^{5α}, and R^{6α} must be selected from the group consisting of —C(O)NH₂, —C(O)NHR², —C(O)NHC(O)R², —C(O)NHSO₂R²;

R^{2β}, R^{3β}, R^{4β}, R^{5β}, and R^{6β} are independently selected from the group consisting of hydrogen, halo, alkoxy, heteroaryloxy, heterocyclic, and heteroaryl, all of which can be optionally substituted by one or more selected from the group consisting of halo, alkyl, lower alkyl, cycloalkyl, acyl, hydroxy, hydroxyalkyl, heteroaryl, heterocyclic, alkoxy, oxo, carboxy, carboxyalkyl, alkoxycarbonyl, and —C(O)N(R²)₂;

R² is independently selected from the group consisting of alkyl, lower alkyl, cycloalkyl, arylalkyl, and heteroarylalkyl, wherein all may be substituted by one or more selected from the group consisting of halo, alkyl, lower alkyl, cycloalkyl, acyl, hydroxy, heterocyclic, alkoxy, oxo, —C(O)NH₂, and —C(O)N(R²)₂;

wherein at least one of R^{2β}, R^{3β}, R^{4β}, R^{5β}, and R^{6β} must be an optionally substituted carbon-carbon linked heterocyclic or heteroaryl;

comprising:

reacting a carbon-linked heteroaryl or heterocyclic substituted benzaldehyde of Formula II

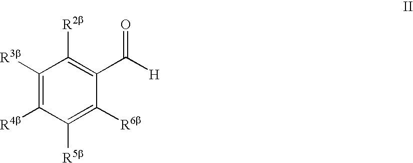

with an acetophenone of Formula III

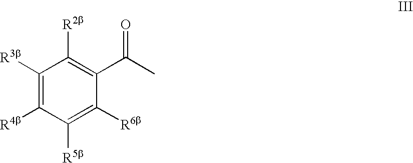

wherein R^{2α}, R^{3α}, R^{4α}, R^{5α}, R^{6α}, R^{2β}, R^{3β}, R^{4β}, R^{5β}, and R^{6β} for Formula II and III are as defined above;

in a solvent or mixture of solvents in the presence of LiOMe.

A 16^{th} embodiment of the invention is a method of manufacturing a compound of Formula I or salts thereof

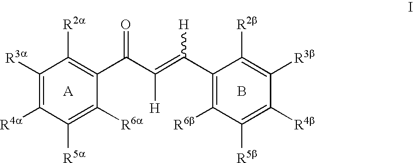

wherein

R^{2α}, R^{3α}, R^{4α}, R^{5α}, and R^{6α} are independently selected from the group consisting of hydrogen, —C(O)NH₂, —C(O)NHR², —C(O)NHC(O)R², —C(O)NHSO₂R²;

wherein at least one of R^{2α}, R^{3α}, R^{4α}, R^{5α}, and R^{6α} must be selected from the group consisting of —C(O)NH₂, —C(O)NHR², —C(O)NHC(O)R², —C(O)NHSO₂R²;

R^{2β}, R^{3β}, R^{4β}, R^{5β}, and R^{6β} are independently selected from the group consisting of hydrogen, halo, alkoxy, heterocyclic, and heteroaryl, all of which can be optionally substituted by one or more selected from the group consisting of lower alkyl, hydroxy, hydroxyalkyl, heteroaryl, heterocyclic, alkoxy, oxo, alkoxycarbonyl, and —C(O)N(R²)₂;

R² is independently selected from the group consisting of lower alkyl, arylalkyl, and heteroarylalkyl, wherein all may be substituted by one or more selected from the group consisting of lower alkyl, heterocyclic, alkoxy, —C(O)NH₂, and —C(O)N(R²)₂;

wherein at least one of R^{2β}, R^{3β}, R^{4β}, R^{5β}, and R^{6β} must be an optionally substituted carbon-carbon linked heterocyclic or heteroaryl; comprising:

reacting a carbon-linked heteroaryl or heterocyclic substituted benzaldehyde of Formula II

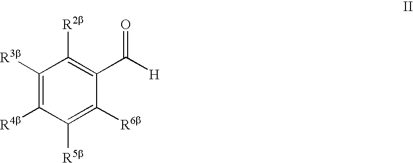

with an acetophenone of Formula III

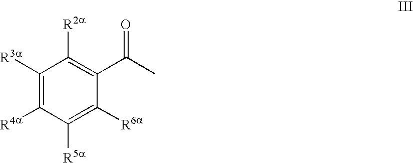

wherein R^{2α}, R^{3α}, R^{4α}, R^{5α}, R^{6α}, R^{2β}, R^{3β}, R^{4β}R^{5β}, and R^{6β} for Formula II and III are as defined above;

in a solvent or mixture of solvents in the presence of LiOMe.

A 17^{th} embodiment of the invention is a method of manufacturing a compound selected from the group consisting of

4-{3E-[4-Methoxy-2-(2-morpholin-4-yl-ethoxy)-5-thiophen-2-yl-phenyl]-acryloyl}-benzamide;

4-[3E-(5-Benzo[b]thiophen-2-yl-2,4-dimethoxy-phenyl)-acryloyl]-benzamide; and

4-{3E-[4-Methoxy-2-(3-morpholin-4-yl-propoxy)-5-thiophen-2-yl-phenyl]-acryloyl}-benzamide;

comprising:

reacting a carbon-linked heteroaryl or heterocyclic substituted benzaldehyde of Formula II

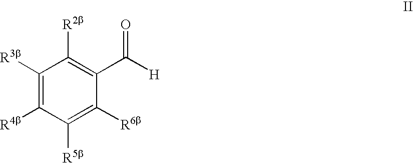

with an acetophenone of Formula III

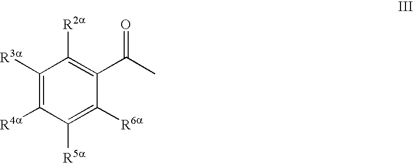

wherein

R^{2α}, R^{3α}, R^{4α}, R^{5α}, and R^{6α} are independently selected from the group consisting of hydrogen, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²), —C(O)NHSO₂NR⁷R⁸, —C(O)NHC(O)R², —C(O)NHSO₂R²;

wherein at least one of R^{2α}, R^{3α}, R^{4α}, R^{5α}, and R^{6α} must be selected from the group consisting of —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², —C(O)NHSO₂R²;

R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;

R⁷ and R⁸ are independently selected from the group consisting of alkyl, alkenyl and aryl and linked together forming a 4- to 12-membered monocyclic, bicylic, tricyclic or benzofused ring;

wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;

wherein at least one of R^{2β}, R^{3β}, R^{4β}, R^{5β}, and R^{6β} must be an optionally substituted carbon-carbon linked heterocyclic or heteroaryl; in a solvent or mixture of solvents in the presence of LiOMe.

The invention may be suitably carried out in water or protic organic solvents such as lower alcohols (e.g. methanol, ethanol, tert-butanol), or in aprotic organic solvents such as ethers (e.g. tetrahydrofuran, dioxane, diethyl ether), liquid amides (e.g. dimethylformamide, hexamethylphosphordiamide), dimethylsulfoxide, hydrocarbons (e.g. toluene, benzene), or mixtures of such solvents, all of which are contemplated by the invention.

Another aspect of the invention is to provide compounds, pharmaceutical compositions and methods to treat diseases usually associated with cardiovascular conditions and/or inflammation. Such diseases include, without limitation, arthritis, asthma, dermatitis, cystic fibrosis, post transplantation late and chronic solid organ rejection, multiple sclerosis, systemic lupus erythematosis, inflammatory bowel diseases, autoimmune diabetes, diabetic retinopathy, diabetic nephropathy, diabetic vasculopathy, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina and small artery disease. Other diseases the invention would be useful for include the treatment of inflammatory skin diseases that are mediated by VCAM-1, as well as human endothelial disorders that are mediated by VCAM-1, which include, but are not limited to, psoriasis, dermatitis, including eczematous dermatitis, Kaposi's sarcoma, multiple sclerosis, as well as proliferative disorders of smooth muscle cells.

Any host organism, including a pateint, mammal, and specifically a human, suffering from any of the above-described conditions can be treated by the administration of a composition comprising an effective amount of the compound of the invention or a pharmaceutically acceptable salt thereof, optionally in a pharmaceutically acceptable carrier or diluent.

The composition can be administered in any desired manner, including oral, topical, parenteral, intravenous, intradermal, intra-articular, intra-synovial, intrathecal, intra-arterial, intracardiac, intramuscular, subcutaneous, intraorbital, intracapsular, intraspinal, intrasternal, topical, transdermal patch, via rectal, vaginal or urethral suppository, peritoneal, percutaneous, nasal spray, surgical implant, internal surgical paint, infusion pump, or via catheter. In one embodiment, the agent and carrier are administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995).

An effective dose for any of the herein described conditions can be readily determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication. Typical systemic dosages for all of the herein described conditions are those ranging from 0.1 mg/kg to 500 mg/kg of body weight per day as a single daily dose or divided daily doses. Preferred dosages for the described conditions range from 5-1500 mg per day. A more particularly preferred dosage for the desired conditions ranges from 25-750 mg per day. Typical dosages for topical application are those ranging from 0.001 to 100% by weight of the active compound.

The compound is administered for a sufficient time period to alleviate the undesired symptoms and the clinical signs associated with the condition being treated.

The active compound is included in the pharmaceutically acceptable carrier or diluent in an amount sufficient to deliver to a patient a therapeutic amount of compound in vivo in the absence of serious toxic effects.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound for systemic delivery is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound can also be mixed with other active materials that do not impair the desired action, or with materials that supplement the desired action. The compounds can also be administered in combination with nonsteroidal antiinflammatories such as ibuprofen, indomethacin, fenoprofen, mefenamic acid, flufenamic acid, sulindac. The compound can also be administered with corticosteriods.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

If administered intravenously, preferred carriers are physiological saline, bacteriostatic water, Cremophor EL™ (BASF, Parsippany, N.J.) or phosphate buffered saline (PBS).

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the compound is then introduced into the container. The container is then swirled by hand to free lipid material from the sides of the container and to disperse lipid aggregates, thereby forming the liposomal suspension.

Suitable vehicles or carriers for topical application can be prepared by conventional techniques, such as lotions, suspensions, ointments, creams, gels, tinctures, sprays, powders, pastes, slow-release transdermal patches, suppositories for application to rectal, vaginal, nasal or oral mucosa. In addition to the other materials listed above for systemic administration, thickening agents, emollients and stabilizers can be used to prepare topical compositions. Examples of thickening agents include petrolatum, beeswax, xanthan gum, or polyethylene, humectants such as sorbitol, emollients such as mineral oil, lanolin and its derivatives, or squalene.

Definitions

A wavy line used as a bond “ [symbol] ”, denotes a bond which can be either the E- or Z-geometric isomer or a mixture of E and Z.

When not used as a bond, the wavy line indicates the point of attachment of the particular substituent.

The terms “alkyl” or “alk”, alone or in combination, unless otherwise specified, refers to a saturated straight or branched primary, secondary, or tertiary hydrocarbon which includes but is not limited to hydrocarbons from 1 to 10 carbon atoms, including, but not limited to methyl, ethyl, propyl, isopropyl, butyl, isobutyl, t-butyl, and sec-butyl. The term “lower alkyl” alone or in combination refers to an alkyl having from 1 to 4 carbon atoms. The alkyl group may be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene et al., Protective Groups in Organic Synthesis, John Wiley & Sons, Second Edition, 1991, hereby incorporated by reference. Specifically included are CF₃ and CH₂CF₃.

The term “alkenyl”, alone or in combination, includes a non-cyclic alkyl of 2 to 10 carbon atoms having one or more unsaturated carbon-carbon bonds. The alkenyl group may be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene et al., Protective Groups in Organic Synthesis, John Wiley & Sons, Second Edition, 1991, hereby incorporated by reference. Specifically included are CF₃ and CH₂CF₃.

The term “alkynyl”, alone or in combination, includes a non-cyclic alkyl of 2 to 10 carbon atoms having one or more triple carbon-carbon bonds, including but not limited to ethynyl and propynyl. The alkynyl group may be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene et al., Protective Groups in Organic Synthesis, John Wiley & Sons, Second Edition, 1991, hereby incorporated by reference. Specifically included are CF₃ and CH₂CF₃.

The terms “carboxy”, “COOH” and “C(O)OH” are used interchangeably.

The terms “alkoxycarbonyl” and “carboalkoxy” are used interchangeably. Used alone or in combination, the terms mean refer to the radical —C(O)OR, wherein R is alkyl as defined herein.

The term “thio”, alone or in combination, means the radical —S—.

The term “thiol”, alone or in combination, means the radical —SH.

The term “hydroxy” or “hydroxyl”, alone or in combination means the radical —OH.

The term “sulfonyl”, alone or in combination means the radical —S(O)₂—.

The term “oxo” refers to an oxygen attached by a double bond (═O).

The term “carbocycle”, alone or in combination, means any stable 3- to 7-membered monocyclic or bicyclic or 7- to 14-membered bicyclic or tricyclic or an up to 26-membered polycyclic carbon ring, any of which may be saturated, partially unsaturated, or aromatic. Examples of such carbocyles include, but are not limited to, cyclopropyl, cyclopentyl, cyclohexyl, phenyl, biphenyl, naphthyl, indanyl, adamantyl, or tetrahydronaphthyl (tetralin).

The term “cycloalkyl”, alone or in combination, includes a saturated or partially unsaturated cyclic alkyl, having from 3 to 10 carbon atoms, including but not limited to mono- or bi-cyclic ring systems such as cyclopropyl, cyclobutyl, cyclopentyl, cyclohexenyl, and cyclohexyl.

The term “aryl”, alone or in combination, includes a carbocyclic aromatic system containing one, two or three rings wherein such rings may be attached together in a pendent manner or may be fused. The “aryl” group can be optionally substituted with one or more of the moieties selected from the group consisting of alkyl, alkenyl, alkynyl, heteroaryl, heterocyclic, carbocycle, alkoxy, oxo, aryloxy, arylalkoxy, cycloalkyl, tetrazolyl, heteroaryloxy; heteroarylalkoxy, carbohydrate, amino acid, amino acid esters, amino acid amides, alditol, halogen, haloalkylthi, haloalkoxy, haloalkyl, hydroxyl, carboxyl, acyl, acyloxy, amino, aminoalkyl, aminoacyl, amido, alkylamino, dialkylamino, arylamino, nitro, cyano, thiol, imide, sulfonic acid, sulfate, sulfonate, sulfonyl, alkylsulfonyl, aminosulfonyl, alkylsulfonylamino, haloalkylsulfonyl, sulfanyl, sulfinyl, sulfamoyl, carboxylic ester, carboxylic acid, amide, phosphonyl, phosphinyl, phosphoryl, thioester, thioether, oxime, hydrazine, carbamate, phosphonic acid, phosphate, phosphonate, phosphinate, sulfonamido, carboxamido, hydroxamic acid, sulfonylimide or any other desired functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1999, hereby incorporated by reference. In addition, adjacent groups on an “aryl” ring may combine to form a 5- to 7-membered saturated or partially unsaturated carbocyclic, aryl, heteroaryl or heterocyclic ring, which in turn may be substituted as above.

The term “heterocyclic”, alone or in combination, includes to a nonaromatic cyclic group that may be partially (containing at least one double bond) or fully saturated and wherein the ring contains at least one heteroatom selected from oxygen, sulfur, nitrogen, or phosphorus. The terms “heteroaryl” or “heteroaromatic”, alone or in combination, refer to an aromatic ring containing at least one heteroatom selected from sulfur, oxygen, nitrogen or phosphorus. The heteroaryl or heterocyclic ring may optionally be substituted by one or more substituent listed as optional substituents for aryl. In addition, adjacent groups on the heteroaryl or heterocyclic ring may combine to form a 5- to 7-membered carbocyclic, aryl, heteroaryl or heterocyclic ring, which in turn may be substituted as above. Nonlimiting examples of heterocylics and heteroaromatics are pyrrolidinyl, tetrahydrofuryl, tetrahydrofuranyl, pyranyl, purinyl, tetrahydropyranyl, piperazinyl, piperidinyl, morpholino, thiomorpholino, tetrahydropyranyl, imidazolyl, pyrolinyl, pyrazolinyl, indolinyl, dioxolanyl, or 1,4-dioxanyl. aziridinyl, furyl, furanyl, pyridyl, pyridinyl, pyridazinyl, pyrimidinyl, benzoxazolyl, 1,2,4-oxadiazolyl, 1,3,4-oxadiazolyl, 1,3,4-thiadiazole, indazolyl, triazinayl, 1,3,5-triazinyl, thienyl, isothiazolyl, imidazolyl, tetrazolyl, pyrazinyl, benzofuranyl, quinolyl, isoquinolyl, benzothienyl, isobenzofuryl, pyrazolyl, indolyl, isoindolyl, benzimidazolyl, purinyl, carbazolyl, oxazolyl, thiazolyl, benzothiazolyl, isothiazolyl, 1,2,4-thiadiazolyl, isooxazolyl, 1,2,4-oxadiazolyl, 1,3,4-oxadiazolyl, pyrrolyl, quinazolinyl, quinoxalinyl, benzoxazolyl, quinolinyl, isoquinolinyl, cinnolinyl, phthalazinyl, xanthinyl, hypoxanthinyl, pyrazole, imidazole, 1,2,3-triazole, 1,2,4-triazole, 1,2,3-oxadiazole, thiazine, pyridazine, triazolopyridinyl or pteridinyl wherein said heteroaryl or heterocyclic group can be optionally substituted with one or more substituent selected from the same substituents as set out above for aryl groups. Functional oxygen and nitrogen groups on the heteroaryl group can be protected as necessary or desired. Suitable protecting groups can include trimethylsilyl, dimethylhexylsilyl, t-butyidimethylsilyl, and t-butyldiphenylsilyl, trityl or substituted trityl, alkyl groups, acyl groups such as acetyl and propionyl, methanesulfonyl, and p-toluenesulfonyl.

The term “aryloxy”, alone or in combination, refers to an aryl group bound to the molecule through an oxygen atom.

The term “heteroaryloxy”, alone or in combination, refers to a heteroaryl group bound to the molecule through an oxygen atom.

The term “aralkyl”, alone or in combination, refers to an aryl group attached to an alkyl group which is attached to the molecule through a carbon atom

The term “aralkoxy”, alone or in combination, refers to an aryl group attached to an alkyl group which is attached to the molecule through an oxygen atom.

The term “heterocyclearalkoxy” refers to a heterocyclic group attached to an aryl group attached to an alkyl-O-group. The heterocyclic, aryl and alkyl groups can be optionally substituted as described above.

The terms “halo” and “halogen”, alone or in combination, refer to chloro, bromo, iodo and fluoro.

The terms “alkoxy” or “alkylthio”, alone or in combination, refers to an alkyl group as defined above bonded through an oxygen linkage (—O—) or a sulfur linkage (—S—), respectively. The terms “lower alkoxy” or “lower alkylthio”, alone or in combination, refers to a lower alkyl group as defined above bonded through an oxygen linkage (—O—) or a sulfur linkage (—S—), respectively.

The term “acyl”, alone or in combination, refers to a group of the formula C(O)R′, wherein R′ is an alkyl, aryl, alkaryl or aralkyl group, or substituted alkyl, aryl, aralkyl or alkaryl, wherein these groups are as defined above.

The term “acetyl”, alone or in combination, refers to the radical —C(O)CH₃.

The term “amino”, alone or in combination, denotes the radical —NH₂, —NH—, or

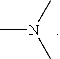

The term “nitro”, alone or in combination, denotes the radical —NO₂.

The term “substituted”, means that one or more hydrogen on the designated atom or substituent is replaced with a selection from the indicated group, provided that the designated atom's normal valency is not exceeded, and the that the substitution results in a stable compound. When a subsitutent is “oxo” (keto) (i.e., ═O), then 2 hydrogens on the atom are replaced.

The term “alditol”, as referred to herein, and unless otherwise specified, refers to a carbohydrate in which the aldehyde or ketone group has been reduced to an alcohol moiety. The alditols of the present invention can also be optionally substituted or deoxygenated at one or more positions. Exemplary substituents include hydrogen, halo, haloalkyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, amino acid, amino acid esters and amides, phosphonyl, phosphinyl, phosphoryl, thioester, thioether, oxime, hydrazine, carbamate, phosphonic acid, and phosphonate. Particular exemplary substituents include amine and halo, particularly fluorine. The substituent or alditol can be either unprotected, or protected as necessary, as known to those skilled in the art, for example, as taught in Greene, et al., Protective Groups in Organic Synthesis, John Wiley and Sons, Second Edition, 1999, hereby incorporated by reference. The alditol may have 3, 4, 5, 6 or 7 carbons. Examples of useful alditols are those derived from reduction of monosaccharides, including specifically those derived from the reduction of pyranose and furanose sugars.

The term “carbohydrate”, as referred to herein, and unless otherwise specified, refers to a compound of carbon, hydrogen and oxygen that contains an aldehyde or ketone group in combination with at least two hydroxyl groups. The carbohydrates of the present invention can also be optionally substituted or deoxygenated at one or more positions. Carbohydrates thus include substituted and unsubstituted monosaccharides, disaccharides, oligosaccharides, and polysaccharides. The saccharide can be an aldose or ketose, and may comprise 3, 4, 5, 6, or 7 carbons. In one embodiment the carbohydrates are monosaccharides. In another embodiment the carbohydrates are pyranose and furanose sugars.

Schemes

The following schemes are nonlimiting embodiments that describe the invention. For the purposes of the schemes, R, R′, R″, and R′″ are considered independent for each scheme and can be any substituent including hydrogen. R, R′, R″, and R′″ can be suitably funtionalized and can represent multiple substitutions. In addition two adjacent R, R′, R″, and R′″ can form a ring. A dashed double bond can be at any location of a ring. X, independently for each scheme, represents Cl, Br, or 1. HetAr represents a suitably substituted heteroaryl. “n” is an integer selected from 0, 1, 2, 3, and 4.

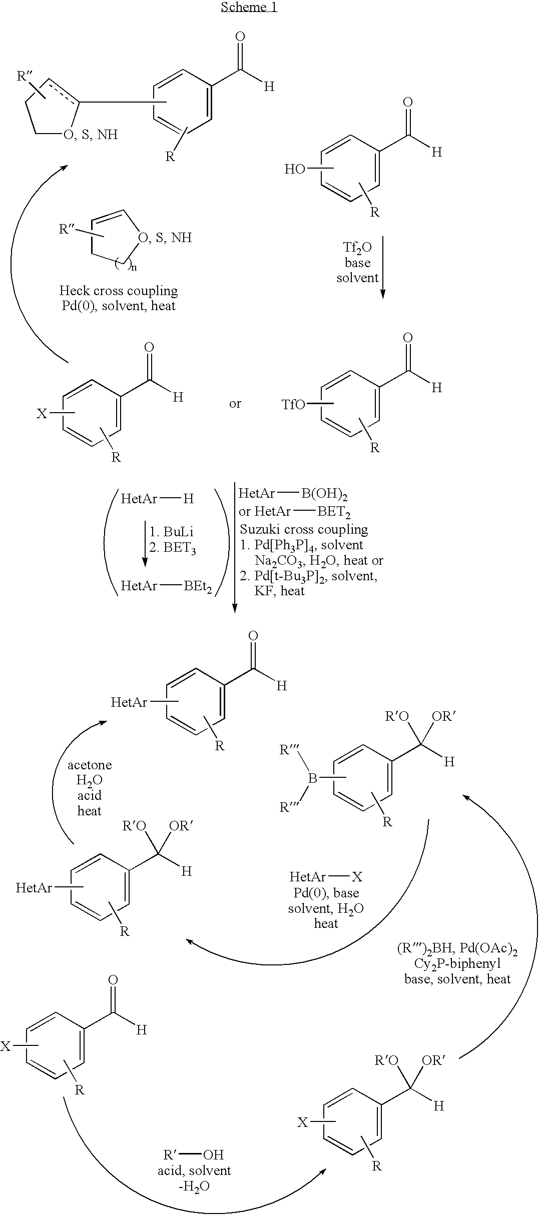

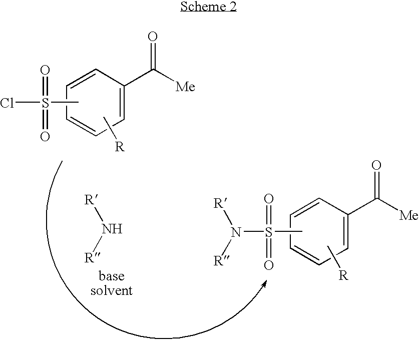

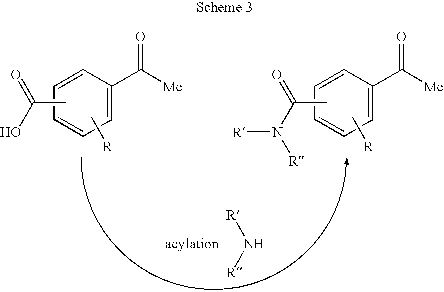

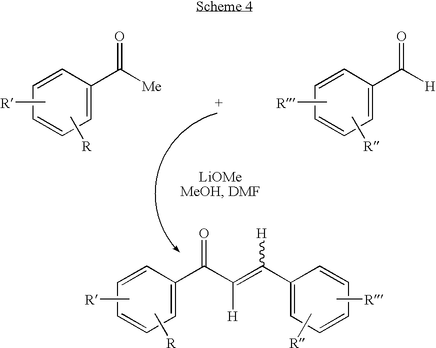

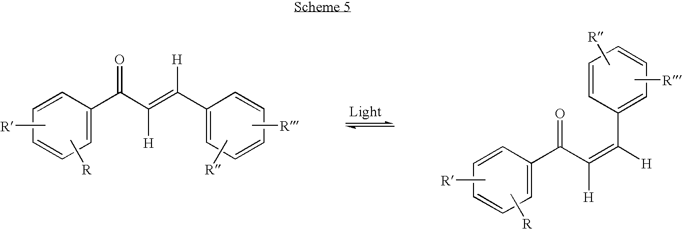

### EXAMPLES

The following examples are provided to illustrate the present invention and are not intended to limit the scope thereof. Those skilled in the art will readily understand that known variations of the conditions and processes of the following preparative procedures can be used to manufacture the desired compounds. The materials required for the embodiments and the examples are known in the literature, readily commercially available, or can be made by known methods from known starting materials by those skilled in the art.

### Example 1

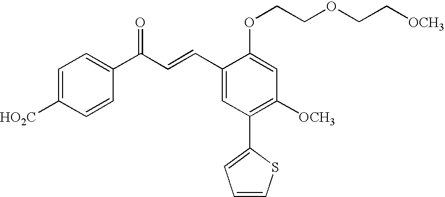

4-(3E-{4-Methoxy-2-[2-(2-methoxyethoxy)ethoxy]-5-thiophen-2-yl-phenyl}-acryloyl)-benzoic Acid

Ex-1A: 2-Hydroxy-4-methoxybenzaldehyde (6.0 g, 39 mmol) was dissolved in dichloromethane (50 mL) and cooled to 0° C. using an ice-water bath. Bromine (6.8 g, 43 mmol) in dichloromethane (2 mL) was added dropwise to the cooled solution and stirred for 2 h at 0° C. The mixture was warmed to room temperature and stirred for an additional 1 h and the resulting yellow precipitate was collected. Recrystallization (ethyl acetate/hexanes) yielded 7.1 g (80%) of 5-bromo-2-hydroxy-4-methoxybenzaldehyde as white needles, m.p. 63-64° C. ¹H-NMR (300 MHz, CDCl₃) δ 11.43 (s, 1H), 9.69 (s, 1H), 7.68 (s, 1H), 6.48 (s, 1H), 3.95 (s, 3H). Anal. Calcd. for C₈H₇BrO₃: C, 41.59; H, 3.05. Found: C, 41.86; H, 3.05.

Ex-1B: 5-Bromo-2-hydroxy-4-methoxybenzaldehyde obtained from Ex-1A (1.5 g, 6.5 mmol) and thiophene-2-boronic acid (0.91 g, 7.1 mmol) were dissolved in tetrahydrofuran (15 mL). Nitrogen was bubbled into the solution for 10 min followed by the sequential addition of potassium fluoride (0.80 g, 14 mmol, spray-dried) and bis(tri-t-butylphosphine)palladium (0) (0.033 g, 0.065 mmol). The solution was immediately heated to 60° C. and aged for 1.5 h. Upon completion, as determined by HPLC, the reaction was diluted with water (25 mL) and extracted with ethyl acetate (3×30 mL). The combined organic extracts were dried over sodium sulfate and concentrated to a brown solid. Silica gel chromatography (ethyl acetate/hexanes, 1:3) gave 1.46 g (97%) of 2-hydroxy-4-methoxy-5-thiophen-2-yl-benzaldehyde as a yellow solid, m.p. 118-119-C. ¹H-NMR (300 MHz, CDCl₃) δ 11.48 (s, 1H), 9.79 (s, 1H), 7.72 (s, 1H), 7.37 (dd, 1H), 7.31 (dd, 1H), 7.08 (dd, 1H), 6.54 (s, 1H), 3.98 (s, 3H). Anal. Calcd. for C₈H₇O₃S: C, 61.52; H, 4.30; S, 13.69. Found: C, 61.12; H, 4.34; S, 13.56.

Ex-1C: To a solution of 2-hydroxy-4-methoxy-5-thiophen-2-yl-benzaldehyde from Ex-1B (0.10 g, 0.43 mmol) in N,N-dimethylformamide (3 mL) was added potassium carbonate (0.18 g, 1.3 mmol) and the resulting yellow slurry was heated to 80° C. Once at 80° C., 1-bromo-2-(2-methoxyethoxy)ethane (0.24 g, 1.3 mmol) was added dropwise in three equal portions with stirring at 1 h intervals. After the last addition, the reaction was stirred for an additional 1 h at 80° C. and cooled to room temperature. The mixture was diluted with water (15 mL) and extracted with ethyl acetate (3×15 mL). The combined organic layers was sequentially washed with a saturated ammonium chloride solution (1×15 mL), water (1×15 mL), and brine (1×15 mL), dried over sodium sulfate, and concentrated to a brown oil. Silica gel chromatography (ethyl acetate/hexanes, 4:1) afforded 0.13 g (87%) of 4-methoxy-2-[2-(2-methoxyethoxy)ethoxy]-5-thiophen-2-yl-benzaldehyde as a pale yellow oil. ¹H-NMR (300 MHz, CDCl₃) δ 10.38 (s, 1H), 8.12 (s, 1H), 7.44 (dd, 1H), 7.30 (dd, 1H), 7.07 (dd, 1H), 6.57 (s, 1H), 4.33 (t, 2H), 4.00 (s, 3H), 3.94 (t, 2H), 3.74 m, 2H), 3.59 (m, 2H), 3.40 (s, 3H). HRMS (EI) Calcd. for C₁₇H₂₀O₅S: 336.1031. Found: 336.1027.

Ex-1D: 4-Methoxy-2-[2-(2-methoxyethoxy)ethoxy]-5-thiophen-2-yl-benzaldehyde obtained from Ex-1C (0.13 g, 0.37 mmol) and 4-acetylbenzoic acid (0.061 g, 0.37 mmol) were dissolved in a tetrahydrofuran-methanol solution (2 mL, 7:3). After complete dissolution, lithium methoxide (0.057 g, 1.5 mmol) was added and the resulting bright orange slurry was stirred in the dark at room temperature for 4 h. Upon completion, as determined by HPLC, the mixture was diluted with water (10 mL), acidified with a 1 N hydrochloric acid solution, and extracted with ethyl acetate (3×15 mL). The combined organic extracts were dried over sodium sulfate and evaporated to dryness. The crude oil was taken up in ethyl alcohol (3 mL) and warmed to 60° C. to obtain complete dissolution and allowed to cool to room temperature. The resulting precipitate was collected and dried in vacuo to yield 0.14 g (85%) of the title compound as a yellow solid, m.p. 145-146-C. ¹H-NMR (300 MHz, DMSO-d₆) δ 8.22 (m, 3H), 8.09 (d, 2H), 8.01 (d, 2H), 7.66 (dd, 1H), 7.52 (d, 1H), 7.13 (dd, 1H), 6.88 (s, 1H), 4.36 (t, 2H), 4.00 (s, 3H), 3.88 (t, 2H), 3.65 (m, 2H), 3.46 (m, 2H), 3.22 (s, 3H). Anal. Calcd. for C₂₆H₂₆NO₇S: C, 64.71; H, 5.43; S, 6.64. Found: C, 64.64; H, 5.44; S, 6.61.

### Example 2

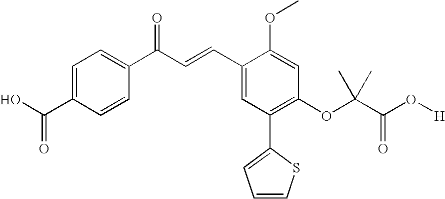

4-{3E-[4-(1-Carboxy-1-methyl-ethoxy)-2-methoxy-5-thiophen-2-yl-phenyl]-acryloyl}-benzoic acid

Ex-2A: 5-Bromo-4-hydroxy-2-methoxy-benzaldehyde was prepared in an analogous fashion as described in Ex-1A using 4-hydroxy-2-methoxybenzaldehyde. The crude solid was slurried in water to remove residual HBr and dried in vacuo to give the bromide as an off-white solid (98%), mp 199-201-C. ¹H-NMR (300 MHz, DMSO-d₆) δ 11.58 (s, 1H), 10.07 (s, 1H), 7.75 (s, 1H), 6.69 (s, 1H), 3.87 (s, 3H). MS (EI) m/z=230 ([M]⁺, 100%). Anal. Calcd. for C₈H₇BrO₃.¼H₂O: C, 40.79; H, 3.21. Found: C, 40.66; H, 3.01.

Ex-2B: 4-Hydroxy-2-methoxy-5-thiophen-2-yl-benzaldehyde was prepared in an analogous fashion as described in Ex-1B. Silica gel chromatography (ethyl acetate/hexanes, 2:1) gave the expected product as a solid (85%), mp 200° C. (dec.). ¹H-NMR (300 MHz, CDCl₃) δ 10.31 (s, 1H), 7.89 (s, 1H), 7.42 (dd, 1H, J=4.8, 1.2 Hz), 7.14-7.19 (m, 2H), 6.59 (s, 1H), 6.14 (brs, 1H), 3.94 (s, 3H). MS (EI) m/z: 234 ([M]⁺, 100%). Anal. Calcd. for C₁₂H₁₀O₃S.H₂O: C, 57.13; H, 4.79; S, 12.71. Found: C, 57.16; H, 4.47; S, 12.48.

Ex-2C: 2-(4-Formyl-5-methoxy-2-thiophen-2-yl-phenoxy)-2-methyl-propionic acid ethyl ester was prepared in an analogous fashion as described in Ex-1C using ethyl 2-bromoisobutyrate. Silica gel chromatography (ethyl acetate/hexanes, 1:1) gave the expected product as a solid (82%), mp 111-113-C. ¹H-NMR (300 MHz, CDCl₃) δ 10.32 (s, 1H), 8.14 (s, 1H), 7.45 (dd, 1H, J=3.7, 1.3 Hz), 7.30 (dd, 1H, J=5.2, 1.3 Hz), 7.07 (dd, 1H, J=5.2, 3.7 Hz), 6.35 (s, 1H), 4.25 (q, 2H, J=7.2 Hz), 3.85 (s, 3H), 1.76 (s, 6H), 1.23 (t, 3H, J=7.2 Hz). MS (EI) m/z=348 ([M]+, 100%). Anal. Calcd. for C₁₈H₂₀O₅S: C, 62.05; H, 5.79; S, 9.20. Found: C, 61.81; H, 5.81; S, 9.12.

Ex-2D: To a solution of 2-(4-formyl-5-methoxy-2-thiophen-2-yl-phenoxy)-2-methylpropionic acid ethyl ester (0.29 g, 0.83 mmol) in a mixture of tetrahydrofuran, water and methanol (9 mL, 4:1:1) was added lithium hydroxide (0.10 g, 2.49 mmol) and the resulting yellow slurry was stirred at rt for 5 h. The mixture was diluted with water (5 mL) and extracted with ethyl acetate (1×5 mL). The aqueous layer was acidified with a 1 N HCl solution and extracted with ethyl acetate (3×15 mL). The combined organic layers was dried over sodium sulfate and concentrated to afford 0.13 g (87%) of 2-(4-formyl-5-methoxy-2-thiophen-2-yl-phenoxy)-2-methyl-propionic acid as a pale green solid, mp 183-184° C. ¹H-NMR (300 MHz, CDCl₃) δ 10.32 (s, 1H), 8.12 (s, 1H), 7.40 (d, 1H, J=3.6 Hz), 7.32 (d, 1H, J=4.8 Hz), 7.08 (dd, 1H, J=4.8, 3.6 Hz), 6.47 (s, 1H), 3.86 (s, 3H), 1.78 (s, 6H). MS (EI) m/z=320 ([M]+, 100%). Anal. Calcd. for C₁₆H₁₆O₅S: C, 59.99; H, 5.03; S, 10.01. Found: C, 60.04; H, 5.26; S, 9.70.

Ex-2E: 2-(4-Formyl-5-methoxy-2-thiophen-2-yl-phenoxy)-2-methyl-propionic acid (Ex-2D, 0.23 g, 0.72 mmol) and 4-acetylbenzoic acid (0.12 g, 0.72 mmol) were dissolved in a dimethylformamide-methanol solution (5 mL, 7:3). After complete dissolution, lithium methoxide (0.11 g, 2.9 mmol) was added and the resulting orange slurry was stirred in the dark at room temperature for 4 h. Upon completion, as determined by HPLC, the mixture was diluted with water (15 mL), acidified with a 1 N hydrochloric acid solution, and extracted with ethyl acetate (4×25 mL). The combined organic extracts were dried over sodium sulfate and evaporated to dryness. The crude oil was taken up in a tetrahydrofuran-heptane solution (5 mL, 10:1) and warmed to 60° C. to obtain complete dissolution and allowed to cool to room temperature. The resulting precipitate was collected on filter paper and dried in vacuo to yield 0.30 g (90%) of the title compound as a dark yellow solid, mp 135-137° C. ¹H-NMR (300 MHz, DMSO-d₆) δ 8.32 (s, 1H), 8.23 (d, 2H, J=8.4 Hz), 8.10 (d, 2H, J=8.4 Hz), 7.99 (d, 2H, J=15.6 Hz), 7.71 (d, 1H, J=3.0 Hz), 7.54 (d, 1H, J=5.1 Hz), 7.14 (dd, 1H, J=5.1, 3.0 Hz), 6.49 (s, 1H), 3.85 (s, 3H), 1.69 (s, 6H). MS (ESI) m/z=467 ([M+H]⁺, 100%). Anal. Calcd. for C₂₅H₂₈O₈S.EtOH: C, 63.27; H, 5.51; S, 6.26. Found: C, 63.40; H, 5.19; S, 6.38.

### Example 3

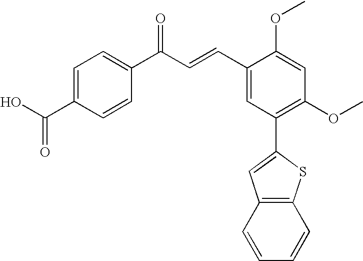

4-[(2E)-3-(5-benzo[b]thien-2-yl-2,4-dimethoxyphenyl)-1-oxo-2-propenyl]-benzoic acid

Ex-3A: A sample of 5-bromo-2,4-dimethoxybenzaldehyde (4.9 g, 20.0 mmol) was dissolved in ethylene glycol dimethyl ether (50 mL). Tetrakis(triphenylphosphine)-palladium(0) (2.32 g, 2 mmol) was added, and the mixture was stirred at room temperature under nitrogen for 5 min. Benzo[b]thiophene-2-boronic acid (4.27 g, 24 mmol) and sodium carbonate solution (2 M, 20 mL) were added. The mixture was stirred at reflux under nitrogen for 24 hours. Upon cooling to room temperature, the mixture was poured into water and extracted with ethyl acetate. The organic phase was dried over sodium sulfate and evaporated. Silica gel chromatography (hexane/ethyl acetate 2:1 then 1:1) gave 4.75 g (83%) of the desired 5-(benzo[b]thien-2-yl)-2,4-dimethoxybenzaldehyde. ¹H NMR (CDCl₃) δ 10.36 (s, 1H), 8.20 (s, 1H), 7.83-7.78 (m, 2H), 7.68 (s, 1H), 7.36-7.27 (m, 2H), 6.54 (s, 1H), 4.06 (s, 3H), 4.00 (s, 3H).

Ex-3AA: (An alternative procedure) 5-bromo-2,4-dimethoxybenzaldehyde (20 g), benzo[b]thiophene-2-boronic acid (16 g) and THF (200 mL) were sequentially charged into a clean reaction vessel fitted with a reflux condenser, mechanical stirrer and nitrogen inlet adapter. Nitrogen was bubbled into the resulting solution for 20 min followed by the sequential addition of KF (10 g), and Pd(^{t}Bu₃P)₂ (0.417 g). The solution was immediately heated to 60° C. and aged for 1.5 h. (Note: The HPLC assay at this point routinely indicated complete consumption of 5-bromo-2,4-dimethoxybenzaldehyde, <0.5 area % of benzo[b]thiophene-2-boronic acid along with 0.5 area % of an unknown (0.55 RRT). These impurities are removed during crystallization.) Upon completion, as determined by HPLC, the reaction was diluted with H₂O (200 mL) and transferred to a separatory funnel containing EtOAc (200 mL) and H₂O (200 mL). The layers were cut and the aqueous layer was extracted with EtOAc (100 mL). The combined organic cuts were filtered through a pre-washed pad of solka floc (5 g). The pad of solka floc and spent catalyst were washed with fresh EtOAc (200 mL) and this wash combined with the batch. The resultant filtrate was batch concentrated and solvent switched to 33 wt % 5-(benzo[b]thien-2-yl)-2,4-dimethoxybenzaldehyde in THF in preparation for crystallization. (Note: The internal temperature during batch concentration should be kept above 45° C. to prevent premature crystallization.) The resulting THF solution of 5-(benzo[b]thien-2-yl)-2,4-dimethoxybenzaldehyde was then charged with heptane (20 mL) and slowly cooled to ambient temperature. Crystallization was then completed with the slow addition of heptane (175 mL) and cooling to 4° C. After aging for 1 h, the batch was filtered and then dried on the filter funnel under a stream of N₂. The semi-wet cake was then transferred to clean trays and dried to a constant weight in the vacuum oven (40° C., 20 in Hg) affording 23.74 g (97% yield) of desired 5-(benzo[b]thien-2-yl)-2,4-dimethoxybenzaldehyde as a light orange crystalline solid, m.p. 134-136° C. HPLC assay of this solid indicated >99.9 LCAP. ¹H-NMR identical as above.

Ex-3AAA: (An alternative procedure) 5-bromo-2,4-dimethoxybenzaldehyde (2150 g, 8.77 mol) was charged to a 72-L reactor followed by THF (13.0 L). The mixture was stirred whilst sparging with argon for 25 minutes. Potassium fluoride (1290 g, 22.20 mol) was added to the reactor and the batch heated to 65° C. under a nitrogen atmosphere, which resulted in a yellow-brown suspension. A solution of Pd(t-Bu₃P)₂ (4.3 g, 8.4 mmol) in THF (110 mL) was sparged with argon for 21 minutes and was then added to the reactor resulting in a dark green suspension. A solution of benzothiophene-2-boronic acid (1634 g, 9.18 mol) in THF (8.6 L) was sparged with argon for 21 minutes, and then added to the hot suspension via an addition funnel. The addition rate was approximately 100 mL/min and the total addition required 85 minutes. During the addition, the suspension became lighter in color and ended as a yellow suspension. After 3.8 L of the boronic acid solution had been added, the suspension began to reflux more vigorously and the addition was suspended until the reflux had returned to normal (approximately 3 minutes). The suspension was maintained at 65° C. for 1 hour after the addition was complete, sampled for HPLC analysis and the heat discontinued.

Water (4.3 L) was added to the cooled batch (<30° C.) and the mixture stirred for 30 minutes and allowed to settle for 25 minutes. The organic phase was washed with saturated sodium chloride solution (6.5 L) for 31 minutes, settled for 20 minutes and the aqueous phase separated. The organic phase was dried with sodium sulfate (1075 g) for 70 minutes. A filter pad was prepared from celite 545 (1075 g) and THF (3.8 L) and the THF discarded. The contents of the 72-L reactor were transferred to the filter pad and the mixture filtered under vacuum. Once the transfer was complete, the reactor was rinsed with THF (3.2 L) and the rinse used to wash the filter cake. The orange organic phases were concentrated in vacuo at 35° C. The wet solid was dried in a vacuum oven (25° C., 30 in Hg) for 15 hours, 32 minutes and weighed. Drying was continued for a further 4 hours at which point the weight was constant and the crude dry product transferred to two amber glass containers and blanketed with nitrogen affording 2542 g (97% of theory) of crude product. Crystallization from THF/heptane as in Ex-3AA results in analytically pure 5-(benzo[b]thien-2-yl)-2,4-dimethoxybenzaldehyde.

Ex-3B: 5-(Benzo[b]thien-2-yl)-2,4-dimethoxybenzaldehyde from Ex-3A, Ex-3AA, or Ex-3AAA (42.3 g), 4-acetylbenzoic acid (22.1 g), MeOH (250 mL) and DMF (600 mL) were sequentially charged into a clean reaction vessel fitted with a mechanical stirrer and nitrogen inlet adapter. After complete dissolution, LiOMe (10.5 g) was added in one portion and the resulting solution was aged at 40° C. for 2 h. Upon completion, as determined by HPLC, the reaction mixture was transferred to a separatory funnel containing cold H₂O (800 mL, precooled to 10 deg C.). An additional 400 mL cold H₂O was used to rinse the reaction vessel and this rinse was also added to the seperatory funnel. The combined aqueous was washed with iPrOAc (500 mL) and then acidified to a pH of 3 with 6 N HCl (ca. 60 mL). The resulting heterogeneous solution was aged for 30 min and then the precipitate was filtered, washed with 70% EtOH (100 mL) and dried on the filter funnel under a stream of N₂ affording desired acid 5 as a crude yellow solid. The crude dry product and THF (260 mL) were charged into a clean reaction vessel fitted with a mechanical stirrer and nitrogen inlet adapter. Heptane (30 mL) was slowly added to the resulting solution over 30 min and then aged resulting in crystallization. Additional heptane (270 mL) was added over 1 h, aged for an additional 1 h and then filtered. The reaction vessel was then rinsed with 70% EtOH (100 mL) and this rinse was added to the filter cake. The wet cake was then transferred to a clean reaction vessel containing 70% EtOH (750 mL) and the resulting heterogeneous mixture was stirred overnight. The product was then filtered, rinsed with fresh 70% EtOH (100 mL) and then dried on the filter funnel under a stream of N₂. The semi-wet cake was then transferred to clean trays and dried to a constant weight in the vacuum oven (40° C., 20 in Hg) affording 52.05 g (87% yield) of desired 4-[(2E)-3-(5-benzo[b]thien-2-yl-2,4-dimethoxyphenyl)-1-oxo-2-propenyl]-benzoic acid as a yellow crystalline solid, m.p. 231-232° C. (dec.). HPLC assay of this solid indicated >99.9 LCAP. ¹H NMR (300 MHz, DMSO-d₆) δ 8.36 (s, 1H), 8.21 (d, 2H), 8.07 (m, 3H), 7.93 (m, 3H), 7.82 (d, 1H), 7.32 (m, 2H), 6.86 (s, 1H), 4.08 (s, 3H), 4.00 (s, 3H). Ex-3BB: 5-(Benzo[b]thien-2-yl)-2,4-dimethoxybenzaldehyde from Ex-3A, Ex-3AA, or Ex-3AAA (1867 g), 4-acetylbenzoic acid (1120 g), MeOH (5.6 L) and DMF (15 L) were charged to a 72-L reactor. Lithium methoxide (485.4 g.) was added to the stirred suspension over approximately 90 minutes in four equal portions. The internal batch temperature increased with each addition of LiOMe, except for the final addition and the overall temperature increased from 17° C. to 30° C. The batch was then heated to 40° C. over 49 minutes and maintained at that temperature for 2 hours, 26 minutes. Ethanol (13.1 L) was added to the very thick yellow slurry and the batch maintained at 40° C. for 2.5 hours and then water (8.4 L) was added over 15 minutes. 6N Hydrochloric acid (2990 mL) was added over 59 minutes. Once addition of the acid was complete, the heat was discontinued and the batch allowed to cool to <30° C. over 14 hours, 34 minutes. The orange suspension was filtered through a 24 inch filter and the reactor rinsed with ethanol (7.5 L, 4 volumes). The rinse was transferred to the filter cake under a stream of nitrogen; the total filtration time was 1 hour, 8 minutes. The filter cake was transferred to glass drying trays and dried in a vacuum oven at 25±5° C. for a total of 27 hours, 27 minutes until constant weight was achieved affording 4-[(2E)-3-(5-benzo[b]thien-2-yl-2,4-dimethoxyphenyl)-1-oxo-2-propenyl]-benzoic acid as an orange solid (2163 g, 78% of theory). The compound of Ex-3 can easily be converted to a salt by those skilled in the art. Suitable salts include but are not limited to arginine (see Ex-67), diethanol amine, lithium, lysine, sodium, meglumine, magnesium, potassium, and triethylamine.

### Example 4

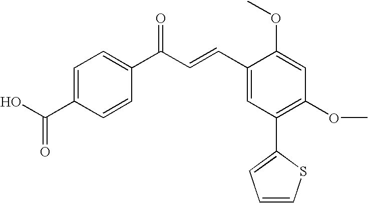

4-[3E-(2,4-Dimethoxy-5-thiophen-2-yl-phenyl)-acryloyl]-benzoic acid

Ex-4A: 5-bromo-2,4-dimethoxybenzaldehyde (20.3 g), thiophene-2-boronic acid (11.6 g) and THF (200 mL) were sequentially charged into a clean reaction vessel fitted with a reflux condenser, mechanical stirrer and nitrogen inlet adapter. Nitrogen was bubbled into the resulting solution for 20 min followed by the sequential addition of KF (10.1 g), and Pd(^{t}Bu₃P)₂ (0.424 g). The solution was immediately heated to 60° C. and aged for 1.5 h. The reaction was diluted with H₂O (200 mL) and transferred to a separatory funnel containing EtOAc (200 mL) and H₂O (200 mL). The layers were cut and the aqueous layer was extracted with EtOAc (100 mL). The combined organic cuts were filtered through a pre-washed pad of solka floc (5 g). The pad of solka floc and spent catalyst were washed with fresh EtOAc (200 mL) and this wash combined with the batch. The resultant filtrate was concentrated to dryness. The crude product was dissolved in THF (38 mL) and crystallized upon heptane (152 mL) addition. The product was filtered and then dried to a constant weight in the vacuum oven (38° C., 20 in Hg) affording 19.32 g (94% yield) of desired 2,4-dimethoxy-5-thiophen-2-yl-benzaldehyde as a light off-white solid, m.p. 125-126° C. ¹H-NMR (300 MHz, CDCl₃): 10.34 (s, 1H), 8.12 (s, 1H), 7.44 (dd, 1H, J=3.5 and 1.5 Hz), 7.31 (dd, 1H, J=5.2 and 1.5 Hz), 7.07 (dd, 1H, J=5.2 and 3.5 Hz), 6.51 (s, 1H), 4.02 (s, 3H), 3.99 (s, 3H).

Ex-4B: 2,4-Dimethoxy-5-thiophen-2-yl-benzaldehyde from Ex-4A (7.81 g), 4-acetylbenzoic acid (4.9 g), MeOH (60 mL) and DMF (150 mL) were sequentially charged into a clean reaction vessel fitted with a stir bar and nitrogen inlet adapter. After complete dissolution LiOMe (4.60 g) was added and the resulting solution was aged for 5 h. The reaction was diluted with H₂O (200 mL) and transferred to a separatory funnel containing iPrOAc (100 mL). The layers were cut and the aqueous layer was acidified to a pH of 1 with 3 N HCl. The resulting precipitate was filtered and then dried on the filter funnel under a stream of N₂. The crude product was then dissolved in THF (60 mL) and crystallized with the addition of heptane (60 mL). The product was filtered and then dried to a constant weight in the vacuum oven affording 8.9 g (75% yield) of the title compound as a yellow solid, m.p. 213-216° C. ¹H-NMR (300 MHz, CDCl₃): 8.20 (d, 2H, J=8.5 Hz), 8.09 (d, 1H, J=16.1 Hz), 8.06 (d, 2H, J=8.5 Hz), 7.85 (s, 1H), 7.52 (d, 1H, J=16.1 Hz), 7.40 (m, 1H), 7.30 (dd, 1H, J=5.2 and 1.7 Hz), 7.08 (dd, 1H, J=5.2 and 3.6 Hz), 6.53 (s, 1H), 3.98 (s, 3H), 3.97 (s, 3H); EIMS m/z=394 (M+). Anal. calc. for C₂₂H₁₈O₅S: C, 66.99; H, 4.60; S, 8.13; found: C, 66.71; H, 4.59; S, 8.10.

### Example 5

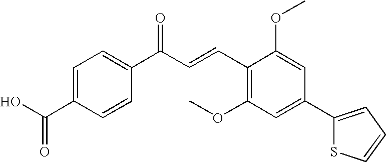

4-[3E-(2,6-Dimethoxy-4-thiophen-2-yl-phenyl)-acryloyl]-benzoic acid

Ex-5A: A solution of 4-hydroxy-2,6-dimethoxy-benzaldehyde (2.3 g, 12.62 mmol) in dichloromethane (25 mL) was cooled to 0° C. and then dimethylamino pyridine (5.6 g, 45.84 mmol) was added in 1 portion. Triflic anhydride (2.5 mL, 14.86 mmol) was then added over 15 min while maintaining an internal temperature below 5° C. The resulting solution was aged for 1 h and then was slowly poured into cold 1 N HCl. The organic phase was dried over magnesium sulfate and concentrated under reduced pressure affording 3.76 g (73%) of the desired methanesulfonic acid 4-formyl-3,5-dimethoxy-phenyl ester.

Ex-5B: A solution of methanesulfonic acid 4-formyl-3,5-dimethoxy-phenyl ester (2.71 g, 8.63 mmol) in 1,4-dioxane (35 mL) was stirred at room temperature under nitrogen for 15 min. Thiophene-2-boronic acid (1.64 g, 12.82 mmol), tetrakis(triphenylphosphine)-palladium(0) (1.02 g, 0.88 mmol) and a potassium phosphate (4.59 g, 21.62 mmol) were then added and the resulting mixture was heated to 95° C. under nitrogen overnight. Upon cooling to room temperature the reaction was diluted with EtOAc and water and the layers were cut. The organic phase was concentrated under reduced pressure. Silica gel chromatography (hexane/ethyl acetate, 4:1) gave 2.14 g (75%) of the desired 2,6-dimethoxy-4-thiophen-2-yl-benzaldehyde product, m.p. 168-170° C. ¹H-NMR (300 MHz, CDCl₃): 10.48 (s, 1H), 7.43 (dd, 1H, J=3.6 and 1.3 Hz), 7.41 (d, 1H, J=5.3 Hz), 7.13 (dd, 1H, J=5.3 and 3.6 Hz), 6.79 (s, 2H), 3.96 (s, 6H).

Ex-5C: The title compound was prepared by condensing 2,6-dimethoxy-4-thiophen-2-yl-benzaldehyde (Ex-5B) and 4-acetylbenzoic acid in a similar manner as described in Ex-3B. Yellow solid, 79% yield, m.p. 256-258° C. ¹H-NMR (300 MHz, d₆-DMSO): 8.11 (d, 1H, J=15.9 Hz), 8.10 (m, 4H), 8.05 (d, 1H, J=15.9 Hz), 7.73 (d, 1H, J=3.6 Hz), 7.61 (d, 1H, J=5.3 Hz), 7.16 (dd, 1H, J=5.3 and 3.6 Hz), 6.95 (s, 2H), 3.98 (s, 6H). MS m/z=394 ([M]+, 100%). HRMS (EI) Calcd. for C₂₂H₁₈O₅S: 394.0875. Found: 394.0877.

### Example 6

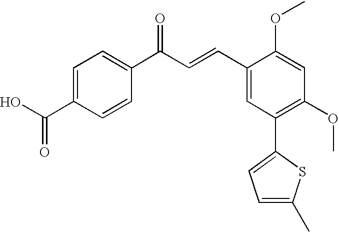

4-{3E-[2,4-Dimethoxy-5-(5-methyl-thiophen-2-yl)-phenyl]-acryloyl}-benzoic acid

Ex-6A: 2,4-Dimethoxy-5-(5-methyl-thiophen-2-yl)-benzaldehyde was prepared from 5-bromo-2,4-dimethoxybenzaldehyde and 5-methyl-thiophene-2-boronic acid in a similar manner as described in Ex-3A, 100% yield. ¹H-NMR (CDCl₃) δ 10.33 (s, 1H), 8.05 (s, 1H), 7.22 (d, J=4 Hz, 1H), 6.72 (d, J=4 Hz, 1H), 6.49 (s, 1H), 4.00 (s, 3H), 3.97 (s, 3H), 2.50 (s, 3H). HMRS (EI) calcd. for C₁₄H₁₄O₃S: 262.0664; found: 262.0665.

Ex-6B: The title compound was prepared by condensing 2,4-dimethoxy-5-(5-methylthiophen-2-yl)-benzaldehyde (Ex-6A) and 4-acetylbenzoic acid in a similar manner as described in Ex-3B. Yellow solid, mp 213-215° C., 27% yield. ¹H-NMR (DMSO-d₆) δ 8.18 (d, J=7 Hz, 2H), 8.17 (s, 1H), 8.00-8.06 (m, 3H), 7.85 (d, J=15 Hz, 1H), 7.42(d, J=4 Hz, 1H), 6.78(m, 2H), 3.96 (s, 3H), 3.95(s, 3H), 2.42 (s, 3H). MS m/z=408 ([M]+, 100%). HMRS (EI) calcd. for C₂₃H₂₀O₅S: 408.1031; found: 408.1023.

### Example 7

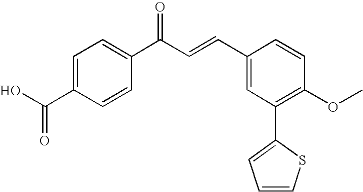

4-[3E-(4-Methoxy-3-thiophen-2-yl-phenyl)-acryloyl]-benzoic acid

Ex-7A: 4-Methoxy-3-(thiophen-2-yl)-benzaldehyde was prepared from 3-bromo-4-methoxybenzaldehyde and thiophene-2-boronic acid in a similar manner as described in Ex-3A. Orange oil, 96% yield. ¹H-NMR (CDCl₃) δ 9.94 (s, 1H), 8.16 (d, J=1.8 Hz, 1H), 7.80 (dd, J=2.4, 8.4 Hz, 1H), 7.57 (dd, J=1.8, 3.6 Hz, 1H), 7.38 (d, J=5.1 Hz, 1H), 7.12 (dd, J=3.6, 5.1 Hz, 1H), 7.09 (d, J=8.4 Hz, 1H), 4.02 (s, 3H). HRMS m/z: calc. 218.0402, found 218.0406.

Ex-7B: The title compound was prepared by condensing 4-methoxy-3-(thiophen-2-yl)-benzaldehyde (Ex-7A) and 4-acetylbenzoic acid in a similar manner as described in Ex-3B. Yellow solid, mp 219-220° C., 71% yield. ¹H-NMR (DMSO-D₆) δ 13.36 (br s, 1H), 8.25-8.31 (m, 3H), 8.11 (d, J=8 Hz, 2H), 7.85-7.98 (m, 3H), 7.78-7.80 (m, 1H), 7.61 (d, J=5 Hz, 1H), 7.25 (d, J=9 Hz, 1H), 7.17 (dd, J=4, 6 Hz, 1H), 3.99 (s, 3H). HRMS m/z=calc. 365.0848, found 365.0833.

### Example 8

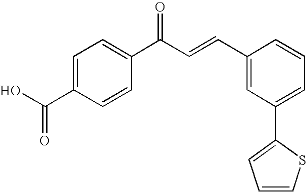

4-[3E-(3-Thiophen-2-yl-phenyl)-acryloyl]-benzoic acid

Ex-8A: 3-(Thiophen-2-yl)-benzaldehyde was prepared from 3-bromobenzaldehyde and thiophene-2-boronic acid in a similar manner as described in Ex-3A. Orange oil, 93% yield. ¹H-NMR (CDCl₃) δ 10.06 (s, 1H), 8.10 (s, 1H), 7.86 (d, J=8.4 Hz, 1H), 7.78 (d, J=7.2 Hz, 1H), 7.55 (dd, J=7.2, 8.4 Hz, 1H), 7.40 (dd, J=1.5, 3.6 Hz, 1H), 7.34 (dd, J=1.5, 5.3 Hz, 1H), 7.11 (dd, J=3.6, 5.3 Hz, 1H). HRMS m/z: calc. 188.0296, found 188.0293.

Ex-8B: The title compound was prepared by condensing 3-(thiophen-2-yl)-benzaldehyde (Ex-8A) and 4-acetylbenzoic acid in a similar manner as described in Ex-3B. Yellow solid, mp 238° C. (dec), 71% yield. ¹H-NMR (DMSO-D₆) δ 13.40 (bs, 1H), 8.29 (d, J=8 Hz, 2H), 8.22 (s, 1H), 8.13 (d, J=8 Hz, 2H), 8.04 (s, 1H), 7.87 (s, 1H), 7.83 (d, J=8 Hz, 1H), 7.73 (d, J=9 Hz, 1H), 7.69 (d, J=4 Hz, 1H), 7.63 (d, J=5 Hz, 1H), 7.52 (t, J=8 Hz, 1H), 7.20 (dd, J=4, 5 Hz, 1H). HRMS m/z=calc. 335.0742, found 335.0749.

### Example 9

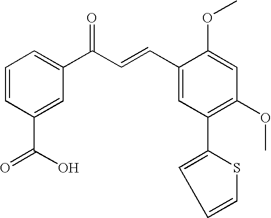

3-[3E-(2,4-Dimethoxy-5-thiophen-2-yl-phenyl)-acryloyl]-benzoic acid

Ex-9: 2,4-dimethoxy-5-(thiophen-2-yl)-benzaldehyde (Ex-4A) and 3-acetylbenzoic acid in a similar manner as described in Ex-3B. Yellow solid, 65% yield, mp 179-182-C. ¹H-NMR (DMSO-d₆) δ 8.54 (s, 1H), 8.39 (d, 1H), 8.25 (s, 1H), 8.15 (d, 1H), 8.04 (d, 1H), 7.90 (d, 1H), 7.67 (m, 2H), 7.48 (d, 1H), 7.09(t, 1H), 6.81 (s, 1H), 3.98 (s, 3H), 3.97 (s, 3H). MS m/z=394 ([M]+, 72%), 363 (100%). Anal. calculated for C₂₂H₁₈O₅S: C, 66.99, H, 4.60, S, 8.13; found C: 66.80, H: 4.60, S: 8.07.

### Example 10

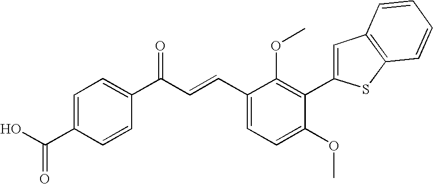

4-[3E-(3-Benzo[b]thiophen-2-yl-2,4-dimethoxy-phenyl)-acryloyl]-benzoic acid

Ex-10A: 3-Benzo[b]thiophen-2-yl-2-hydroxy-4-methoxy-benzaldehyde was prepared through Suzuki coupling as described in Ex-3A using 3-bromo-2-hydroxy-4-methoxybenzaldehyde. ¹H-NMR (CDCl₃) δ 12.08 (s, 1H), 9.80 (s, 1H), 7.80-7.87 (m, 2H), 7.70 (s, 1H), 7.56 (d, J=9 Hz, 1H), 7.31-7.35 (m, 2H), 6.71 (d, J=9 Hz, 1H), 3.97 (s, 3H). HRMS m/z: calc. 284.0507, found 284.0502.

Ex-10B: 3-Benzo[b]thiophen-2-yl-2-hydroxy-4-methoxy-benzaldehyde (Ex-10A, 57.4 mg, 0.202 mmol) was dissolved in acetone (5 mL) and potassium carbonate (31 mg, 0.22 mmol) was added. Methyl iodide (25 uL, 0.40 mmol) was added and the solution was heated to reflux for 3.5 h. After cooling, the crude reaction mix was concentrated on the rotavap. The resulting residue was taken up in 10 mL of a 1:9 mix of saturated, aqueous NH₄Cl to water and extracted with EtOAc (2×1 5 mL). The organic phase was dried over sodium sulfate, filtered, and concentrated to provide 58.5 mg of 3-benzo[b]thiophen-2-yl-2,4-dimethoxy-benzaldehyde as an orange, oily residue which was used without further purification, 97% yield. ¹H-NMR (CDCl₃) δ 10.31 (s, 1H), 7.92 (d, J=9 Hz, 1H), 7.81-7.88 (m, 2H), 7.56 (d, 1H), 7.33-7.39 (m, 2H), 6.88 (d, J=9 Hz, 1H), 3.91 (s, 3H), 3.64 (s, 3H).

Ex-10C: The title compound was prepared by condensing 3-benzo[b]thiophen-2-yl-2,4-dimethoxy-benzaldehyde (Ex-10B) and 4-acetylbenzoic acid in a similar manner as described in Ex-3B. Yellow solid, mp 237° C. (dec.), 64% yield. ¹H-NMR (DMSO-d₆) δ 13.37 (bs, 1H), 8.20-8.25 (m, 3H), 8.11 (d, J=8 Hz, 2H), 8.02 (d, J=8 Hz, 1H), 7.96 (d, J=9 Hz, 2H), 7.88-7.91 (m, 1H), 7.65 (s, 1H), 7.35-7.43 (m, 2H), 7.14 (d, J=9 Hz, 1H), 3.90 (s, 3H), 3.53 (s, 3H). HRMS m/z=calc. 445.1110, found 445.1112.

### Example 11

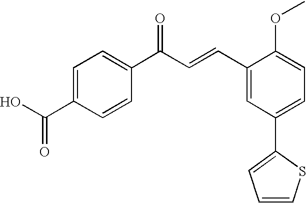

4-[3E-(2-Methoxy-5-thiophen-2-yl-phenyl)-acryloyl]-benzoic acid

Ex-11A: 2-Methoxy-5-(thiophen-2-yl)-benzaldehyde was prepared from 5-bromo-2-methoxybenzaldehyde and thiophene-2-boronic acid in a similar manner as described in Ex-3A. ¹H NMR (CDCl₃) δ 10.49 (s, 1H), 8.07 (d, J=3 Hz, 1H), 7.79 (dd, J=3, 9.0 Hz, 1H), 7.28-7.26 (m, 2H), 7.09-7.06 (m, 1H), 7.02 (d, J=9 Hz, 1H), 3.97 (s, 3H).

Ex-11B: The title compound was prepared by condensing 2-methoxy-5-(thiophen-2-yl)-benzaldehyde (Ex-11A) and 4-acetylbenzoic acid in a similar manner as described in Ex-3B. Yellow solid, mp 195-196° C. ¹H-NMR (DMSO-d₆) δ 8.23-8.20 (m, 3H), 8.08-7.96 (m, 4H), 7.67 (dd, J=2.1, 6.8 Hz, 1H), 7.55 (d, J=3.8 Hz, 1H), 7.49 (d, J=5.1 Hz, 1H), 7.16-7.11 (m, 2H), 3.90 (s, 3H). MS m/z=364 (M+, 100%).

### Example 12

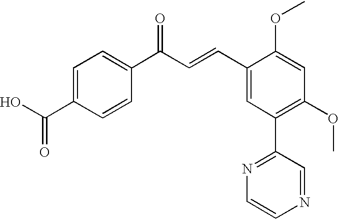

4-[3E-(2,4-Dimethoxy-5-pyrazin-2-yl-phenyl)-acryloyl]-benzoic acid

Ex-12A: 5-Bromo-2,4-dimethoxybenzaldehyde (4.92 g, 20.1 mmol) was dissolved in benzene (41 mL). Ethylene glycol (3 mL, 54 mmol) and p-toluenesulfonic acid (25 mg, 0.13 mmol) were added and the solution was refluxed with a Dean-Stark trap attached. After 6 h, the reaction was cooled and washed with water (1×20 mL), saturated, aqueous NaHCO₃ (1×20 mL), and water (1×20 mL). The organic phase was dried over sodium sulfate, filtered, concentrated, and dried to provide 5.32 g of 2-(5-bromo-2,4-dimethoxy-phenyl)-[1,3]dioxolane as a faint yellow oil which solidified upon standing (92% yield).

¹H-NMR (CDCl₃) δ 7.67 (s, 1H), 6.47 (s, 1H), 6.06 (s, 1H), 4.11-4.13 (m, 2H), 3.98-4.03 (m, 2H), 3.91 (s, 3H), 3.87 (s, 3H). HRMS (ES+) Calcd. for C₁₁H₁₃BrO₄: 289.0075. Found: 289.0077.

Ex-12B: 2-(5-Bromo-2,4-dimethoxy-phenyl)-[1,3]dioxolane (Ex-12A, 4.78 g, 10.5 mmol) was dissolved in dioxane (75 mL) and the solution was purged with nitrogen for 15 min. Pd(OAc)₂ (188 mg, 0.84 mmol), Et₃N (6.91 mL, 49.6 mmol), and 2-(dicyclohexylphosphino)biphenyl (1.16 g, 3.31 mmol) were added. 4,4,5,5-Tetramethyl-[1,3,2]dioxaborolane (3.6 mL, 24.8 mmol) was added slowly, accompanied by gas evolution and the darkening of the reaction solution. The solution was heated at reflux for 2.5 h and then cooled. Saturated, aqueous NH₄Cl (60 mL) and water (20 mL) were added and the solution extracted with EtOAc (1×100 mL). The organic phase was dried over sodium sulfate, filtered, and concentrated to a dark oil. The oil was purified via silica gel chromatography (1:1 EtOAc/hexanes after a column pre-wash of 5% Et₃N in 1:1 EtOAc/hexanes) to provide 3.27 g of 2-(5-[1,3]dioxolan-2-yl-2,4-dimethoxy-phenyl)-4,4,5,5-tetramethyl-[1,3,2]dioxaborolane as a yellow solid (with some starting borolane present), 59% yield. ¹H-NMR (CDCl₃) δ 7.85 (s, 1H), 6.39 (s, 1H), 6.07 (s, 1H), 4.13-4.18 (m, 2H), 3.98-4.02 (m, 2H), 3.89 (s, 3H), 3.84 (s, 3H), 1.33 (s, 9H).

Ex-12C: 2-(5-[1,3]Dioxolan-2-yl-2,4-dimethoxy-phenyl)-4,4,5,5-tetramethyl-[1,3,2]dioxaborolane (Ex-12B, 2.22 g, 6.60 mmol, containing borolane impurity) was dissolved in DME (60 mL) and 2-iodopyrazine (0.59 mL, 6.0 mmol) was added. 2M aqueous Na₂CO₃ (17.8 mL, 35.6 mmol) was added and the mixture was purged with nitrogen for 20 min. Tetrakis(triphenylphosphine)palladium(0) (0.69 g, 0.60 mmol) was added and the mixture was heated at reflux for 2.5 h. After cooling, water (50 mL) was added and the mixture was extracted with CH₂Cl₂ (2×30 mL). The organic phase was washed with brine (1×20 mL), dried over sodium sulfate, filtered, and concentrated. Purification of the resulting yellow-orange solids via silica chromatography (50-80% EtOAc/hexanes) provided 1.02 g of 2-(5-[1,3]dioxolan-2-yl-2,4-dimethoxy-phenyl)-pyrazine as a yellow solid (59% yield). ¹H-NMR (CDCl₃) δ 9.10 (d, J=2 Hz, 1H), 8.61 (m, 1H), 8.39 (d, J=3 Hz, 1H), 8.07 (s, 1H), 6.57 (s, 1H), 6.14 (s, 1H), 4.13-4.18 (m, 2H), 4.01-4.05 (m, 2H), 3.95 (s, 3H), 3.93 (s, 3H).

Ex-12D: 2-(5-[1,3]Dioxolan-2-yl-2,4-dimethoxy-phenyl)-pyrazine (1.02 g, 3.54 mmol) was dissolved in acetone and p-toluenesulfonic acid (100 mg, 0.53 mmol) and water (5 mL) were added. The solution was stirred for 3 h at room temperature, then concentrated on the rotavap. The resulting mixture was diluted with water (50 mL) and extracted with EtOAc (3×100 mL). The organic phase was washed with 25% saturated aqueous NaHCO₃, dried over sodium sulfate, filtered, and concentrated. Drying gave 0.30 g of 2,4-dimethoxy-5-pyrazin-2-yl-benzaldehyde as a yellow solid (18% yield). ¹H-NMR (CDCl₃) δ 10.35 (s, 1H), 9.06 (d, J=2 Hz, 1H), 8.63-8.65 (m, 1H), 8.45 (d, J=2 Hz, 1H), 8.39 (s, 1H), 6.56 (s, 1H), 4.03 (s, 3H), 4.01 (s, 3H). HRMS m/z: calc. 244.0848, found 244.0853.

Ex-12E: The title compound was prepared by condensing 2,4-dimethoxy-5-pyrazin-2-yl-benzaldehyde (Ex-12D) and 4-acetylbenzoic acid in a similar manner as described in Ex-3B. Yellow solid, mp 238° C. (dec.), 4% yield. ¹H-NMR (DMSO-D₆) δ 9.04 (d, J=2 Hz, 1H), 8.75-8.76 (m, 1H), 8.56 (d, J=2 Hz, 1H), 8.32 (s, 1H), 8.19 (d, J=9 Hz, 2H), 8.05-8.11 (m, 3H), 7.83 (d, J=16 Hz, 1H), 6.90 (s, 1H), 4.05 (s, 3H), 4.00 (s, 3H). HRMS m/z=calc. 391.1294, found 391.1313.

### Example 13

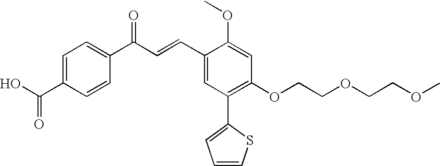

4-(3E-{2-Methoxy-4-[2-(2-methoxy-ethoxy)-ethoxy]-5-thiophen-2-yl-phenyl}-acryloyl)-benzoic acid

Ex-13A: To a solution of 4-hydroxy-2-methoxy-5-thiophen-2-yl-benzaldehyde (Ex-2B, 0.50 g, 2.14 mmol) and tri(ethylene glycol) monomethyl ether (0.38 g, 3.2 mmol) in tetrahydrofuran (20 mL) was added triphenylphosphine (0.84 g, 3.2 mmol) and the resulting mixture was cooled to 0° C. Diethyl azodicarboxylate (0.55 g, 3.2 mmol) was then added drop wise, stirred at 0° C. for 30 min, and allowed to warm to rt. The solution was stirred for an additional 24 and concentrated under reduced pressure to a brown oil. Silica gel chromatography (ethyl acetate/hexanes, 8:1) afforded 0.31 g (45%) of the expected 2-methoxy-4-[2-(2-methoxy-ethoxy)-ethoxy]-5-thiophen-2-yl-benzaldehyde as a viscous clear oil. ¹H-NMR (300 MHz, CDCl₃) δ 10.34 (s, 1H), 8.13 (s, 1H), 7.48 (d, 1H, J=3.6 Hz), 7.30 (t, 1H, J=5.1 Hz), 7.06 (dd, 1H, J=5.1, 3.6 Hz), 6.56 (s, 1H), 4.34 (t, 2H, J=5.1 Hz), 3.94 (t, 2H, J=5.1 Hz), 3.96 (s, 3H), 3.72-3.75 (m, 2H), 3.56-3.59 (m, 2H), 3.39 (s, 3H). MS (ESI) m/z=337 ([M+H]⁺, 100%). HRMS (EI) Calcd. for C₁₇H₂₀O₅S: 336.1031. Found: 336.1028.

Ex-13B: The title compound was prepared by condensing 2-methoxy-4-[2-(2-methoxy-ethoxy)-ethoxy]-5-thiophen-2-yl-benzaldehyde (Ex-13A) and 4-acetylbenzoic acid in a similar manner as described in Ex-3B. Yellow solid, mp 174-175° C., 61% yield. ¹H-NMR (300 MHz, DMSO-d₆) δ 8.28 (s, 1H), 8.23 (d, 2H, J=8.1 Hz), 8.05-8.11 (m, 3H), 7.91 (d, 1H, J=15.3 Hz), 7.72 (d, 1H, J=2.7 Hz), 7.52 (d, 1H, J=4.2 Hz), 7.11-7.15 (m, 1H), 6.86 (s, 1H), 4.39 (t, 2H, J=3.9 Hz), 3.99 (s, 3H), 3.89 (t, 2H, J=3.9 Hz), 3.64 (t, 2H, J=3.9 Hz), 3.48 (t, 2H, J=3.9 Hz), 3.25 (s, 3H). MS (ESI) m/z=483 ([M+H]⁺, 100%). Anal. Calcd. for C₂₆H₂₆O₇S: C, 64.71; H, 5.43; S, 6.64. Found: C, 64.43; H, 5.34; S, 6.54.

### Example 14

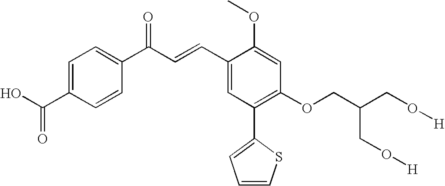

4-{3E-[4-(3-Hydroxy-2-hydroxymethyl-propoxy)-2-methoxy-5-thiophen-2-yl-phenyl]-acryloyl}-benzoic acid

Ex-14A: To a solution of 3-(tert-butyl-dimethyl-silanyloxy)-2-(tert-butyl-dimethyl-silanyloxymethyl)-propan-1-ol (25.0 g, 74.3 mmol) and triethylamine (22.6 g, 223 mmol) in dichloromethane (150 mL) at 0° C. was added mesyl chloride (12.8 g, 111 mmol) and the resulting slurry was stirred at 0° C. for 15 min and allowed to warm to rt. The solution was stirred for an additional 3 h at rt and diluted with water (130 mL) and ethyl acetate (350 mL). The layers were separated and the aqueous was extracted with ethyl acetate (1×150 mL). The combined organic extracts were washed with a saturated sodium bicarbonate (1×200 mL), a 50% sodium chloride solution (2×200 mL), dried over sodium sulfate and concentrated to afford 29.5 g (97%) of the expected methanesulfonic acid 3-(tert-butyl-dimethyl-silanyloxy)-2-(tert-butyl-dimethyl-silanyloxymethyl)-propyl ester as a yellow oil, 97% yield. ¹H-NMR (300 MHz, CDCl₃) δ 4.29 (d, 2H, J=5.7 Hz), 3.61-3.68 (m, 4H), 2.99 (s, 3H), 2.04-2.11 (m, 1H), 0.88 (s, 18H), 0.049 (s, 12H). HRMS (ESI) Calcd. for C₁₇H₄₀O₅SSi₂: 413.2213. Found: 413.2226.

Ex-14B: 4-[3-(tert-Butyl-dimethyl-silanyloxy)-2-(tert-butyl-dimethyl-silanyloxymethyl)-propoxy]-2-methoxy-5-thiophen-2-yl-benzaldehyde was prepared in an analogous fashion as described in Ex-1C using methanesulfonic acid 3-(tert-butyl-dimethyl-silanyloxy)-2-(tert-butyl-dimethyl-silanyloxymethyl)-propyl ester (Ex-14A). Silica gel chromatography (ethyl acetate/hexanes, 1:6) gave the expected product as a pale green solid, 90% yield. ¹H-NMR (300 MHz, CDCl₃) δ 10.34 (s, 1H), 8.13 (s, 1H), 7.41 (dd, 1H, J=3.6, 1.2 Hz), 7.28 (dd, 1H, J=5.1, 1.2 Hz), 7.05 (dd, 1H, J=5.1, 3.6 Hz), 6.54 (s, 1H), 4.22 (d, 2H, J=5.7 Hz), 3.96 (s, 3H), 3.80 (d, 4H, J=5.7 Hz), 2.33 (pentet, 1H, J=5.7 Hz), 0.88 (s, 18H), 0.012 (s, 12H). MS (ESI) m/z=551 ([M+H]⁺, 100%). HRMS (EI) Calcd. for C₂₈H₄₆O₅SSi₂: 550.2604. Found: 550.2593.

Ex-14C: To a solution of 4-[3-(tert-butyl-dimethyl-silanyloxy)-2-(tert-butyl-dimethyl-silanyloxymethyl)-propoxy]-2-methoxy-5-thiophen-2-yl-benzaldehyde (Ex-14B, 0.78 g, 1.41 mmol) in tetrahydrofuran (5 mL) was added tetrabutylammonium fluoride (1 M in tetrahydrofuran, 3.0 mL, 2.9 mmol) and the mixture was stirred at rt for 30 min. The reaction was diluted with ethyl acetate (50 mL) and washed with a 50% ammonium chloride solution (1×30 mL), water (2×30 mL), brine (1×30 mL), dried over sodium sulfate and concentrated to a crude yellow solid. Silica gel chromatography afforded 0.37 g (99%) of the expected 4-(3-hydroxy-2-hydroxymethyl-propoxy)-2-methoxy-5-thiophen-2-yl-benzaldehyde as a pale yellow solid, 90% yield, mp 144-145° C. ¹H-NMR (300 MHz, CDCl₃) δ 10.33 (s, 1H), 8.10 (s, 1H), 7.38 (dd, 1H, J=3.6, 1.5 Hz), 7.30 (dd, 1H, J=5.1, 1.5 Hz), 7.07 (dd, 1H, J=5.1, 3.6 Hz), 6.59 (s, 1H), 4.35 (d, 2H, J=6.0 Hz), 4.02 (t, 4H, J=4.8 Hz), 3.96 (s, 3H), 2.33 (pentet, 1H, J=6.0 Hz), 1.89 (t, 2H, J=4.8 Hz). MS (ESI) m/z=323 ([M+H]⁺, 100%). Anal. Calcd. for C₁₆H₁₈O₅S: C, 59.61; H, 5.63; S, 9.95. Found: C, 59.34; H, 5.75; S, 9.82.

Ex-14D: The title compound was prepared by condensing 4-(3-hydroxy-2-hydroxymethyl-propoxy)-2-methoxy-5-thiophen-2-yl-benzaldehyde (Ex-14C) and 4-acetylbenzoic acid in a similar manner as described in Ex-3B. Yellow solid, mp 199-201° C., 60% yield. ¹H-NMR (300 MHz, DMSO-d₆) δ 8.31 (s, 1H), 8.23 (d, 2H, J=8.7 Hz), 8.06-8.11 (m, 3H), 7.93 (d, 1H, J=15.0 Hz), 7.71 (d, 1H, J=3.3 Hz), 7.54 (d, 1H, J=5.1 Hz), 7.13-7.16 (m, 1H), 6.87 (s, 1H), 4.62 (brs, 2H), 4.27 (d, 2H, J=5.1 Hz), 4.00 (s, 3H), 3.62 (brs, 4H), 2.11-2.15 (m, 1H). MS (ESI) m/z=469 ([M+H]⁺, 100%). Anal. Calcd. for C₂₅H₂₄O₇S.¼H₂O: C, 63.48; H, 5.22; S, 6.78. Found: C, 63.45; H, 5.29; S, 6.61.

### Example 15

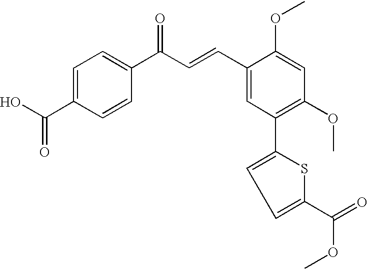

5-{5-[3-(4-Carboxy-phenyl)-3-oxo-E-propenyl]-2,4-dimethoxy-phenyl}-thiophene-2-carboxylic acid methyl ester

Ex-15A: 5-(5-Formyl-2,4-dimethoxy-phenyl)-thiophene-2-carboxylic acid methyl ester was prepared starting from 5-bromo-thiophene-2-carboxylic acid methyl ester in a similar manner as described in Ex-12A through Ex-12D. Yellow solid, 18% yield. ¹H-NMR (CDCl₃) δ 10.32 (s, 1H), 8.16 (s, 1H), 7.74 (d, J=4.4 Hz, 1H), 7.42 (d, J=4.4 Hz, 1H), 6.51 (s, 1H), 4.05 (s, 3H), 3.98 (s, 3H), 3.90 (s, 3H). HRMS (ES+) Calcd. for C₁₅H₁₄O₅S: 307.0640. Found: 307.0630.

Ex-15B: 4-Acetylbenzoic acid (24 mg, 0.15 mmol) and 5-(5-formyl-2,4-dimethoxyphenyl)-thiophene-2-carboxylic acid methyl ester (Ex-15A, 46 mg, 0.15 mmol) were dissolved in DMF (4 mL). Lithium methoxide, 1M in methanol (0.29 mL) was added and the solution stirred at room temperature overnight. The reaction solution was poured into cold 1N HCl (3 mL) and extracted with EtOAc (3×20 mL); the organic phase was washed with brine (1×10 mL), dried over sodium sulfate, filtered, and concentrated. The resulting orange residue was purified via silica gel chromatography (0-10% MeOH/CH₂Cl₂) to provide 89 mg of yellow solid which still contained DMF. The solid was slurried in EtOH for several hours, filtered, and dried to provide 31 mg of final product as a yellow solid (47% yield). ¹H-NMR (DMSO-d₆) δ 8.47 (s, 1H), 8.23 (d, J=9 Hz, 2H), 8.01-8.11 (m, 4H), 7.89 (d, J=4 Hz, 1H), 7.82 (d, J=4 Hz, 1H), 6.90 (s, 1H), 4.09 (s, 3H), 4.03 (s, 3H), 3.84 (s, 3H). HRMS (ES+) Calcd. for C₂₄H₂₀O₇S: 453.1008. Found: 453.1020.

### Example 16

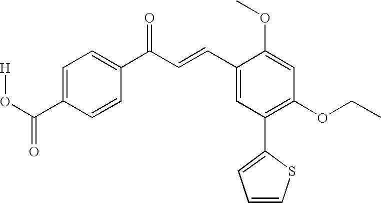

4-[3E-(4-Ethoxy-2-methoxy-5-thiophen-2-yl-phenyl)-acryloyl]-benzoic acid

Ex-16A: Reaction of 4-hydroxy-2-methoxy-5-thiophen-2-yl-benzaldehyde (Ex-2B) and (2-ethoxymethyl-5-hydroxymethyl-[1,3]dioxolan-4-yl)methanol was preformed under the Mitsunobu condition using triphenylphosphine and diethyl azodicarboxylate in THF. However, the expected product, 4-(2-ethoxymethyl-5-hydroxymethyl-[1,3]dioxolan-4-ylmethoxy)-2-methoxy-5-thiophen-2-yl-benzaldehyde, was not obtained. Instead, 4-ethoxy-2-methoxy-5-thiophen-2-yl-benzaldehyde was formed via cleavage of the cyclic ethyl orthoformate group under the reaction conditions. Silica gel chromatography (ethyl acetate/hexanes, 1:2) gave 0.16 g (90%) of 4-ethoxy-2-methoxy-5-thiophen-2-yl-benzaldehyde, mp 101-103-C. ¹H-NMR (300 MHz, CDCl₃) δ 10.33 (s, 1H), 8.15 (s, 1H), 7.48 (d, 1H, J=3.6 Hz), 7.29 (d, 1H, J=5.2 Hz), 7.07 (dd, 1H, J=5.2, 3.6 Hz), 6.50 (s, 1H), 4.25 (q, 2H, J=7.2 Hz), 3.97 (s, 3H), 1.59 (t, 3H, J=7.2 Hz). MS (EI) m/z=262 ([M]+, 100%). HMRS (EI) Calcd. for C₁₄H₁₄O₃S: 262.0664. Found: 262.0667.

Ex-16B: The title compound was prepared by condensing 4-ethoxy-2-methoxy-5-thiophen-2-yl-benzaldehyde (Ex-16A) and 4-acetylbenzoic acid in a similar manner as described in Ex-3B. Yellow solid, mp 210-212° C., 76% yield. ¹H-NMR (300 MHz, DMSO-d₆) δ 8.31 (s, 1H), 8.23 (d, 2H, J=9.0 Hz), 8.06-8.11 (m, 3H), 7.92 (d, 1H, J=16.2 Hz), 7.71 (d, 1H, J=3.9 Hz), 7.52 (d, 1H, J=5.1 Hz), 7.13 (dd, 1H, J=5.1, 3.9 Hz), 6.82 (s, 1H), 4.33 (q, 2H, J=6.1 Hz), 3.99 (s, 3H), 1.48 (t, 3H, J=6.1 Hz). MS (ESI) m/z=409 ([M+H]⁺, 100%). Anal. Calcd. for C₂₃H₂₀O₅S.½H₂O: C, 66.17; H, 5.07; S, 7.68. Found: C, 65.88; H, 5.24; S, 7.36.

### Example 17

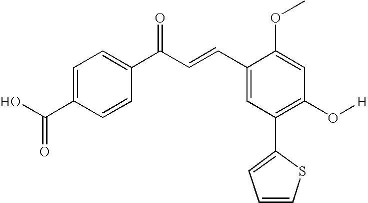

4-[3E-(4-Hydroxy-2-methoxy-5-thiophen-2-yl-phenyl)-acryloyl]-benzoic acid

Ex-17: 4-Hydroxy-2-methoxy-5-thiophen-2-yl-benzaldehyde (Ex-2B, 0.30 g, 0.86 mmol) and 4-acetylbenzoic acid (0.13 g, 0.86 mmol) were dissolved in a dimethylformamide-methanol solution (6 mL, 7:3). After complete dissolution, lithium methoxide (0.12 g, 3.3 mmol) was added and the resulting red slurry was stirred in the dark at room temperature for 18 h. The mixture was diluted with water (15 mL), acidified with a 1 N hydrochloric acid solution, and extracted with ethyl acetate (4×25 mL). The combined organic extracts were dried over sodium sulfate and evaporated to dryness. The crude oil was subjected to silica gel chromatography (CH₂Cl₂:MeOH, 20:1) to yield an orange solid containing residual amounts of starting acid. The solid was taken up in ethyl alcohol (5 mL) to remove acid impurity and the resulting precipitate was collected on filter paper and dried in vacuo to yield 0.010 g (5%) of the title compound as an orange solid, mp 243° C. (dec). ¹H-NMR (300 MHz, DMSO-d₆) δ 8.18-8.23 (m, 3H), 8.06-8.09 (m, 2H), 8.02 (s, 1H), 7.85 (d, 1H, J=15.6 Hz), 7.68 (d, 1H, J=3.6 Hz), 7.47 (d, 1H, J=5.1 Hz), 7.11 (dd, 1H, J=5.1, 3.6 Hz), 6.67 (s, 1H), 4.13 (s, 1H), 3.89 (s, 3H). MS (ESI) m/z=381 ([M+H]⁺, 100%). HRMS (ESI) Calcd. for C₂₁H₁₆O₅S: 381.0796. Found: 381.0800.

### Example 18

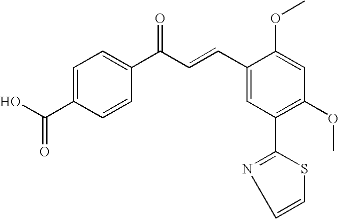

4-[3E-(2,4-Dimethoxy-5-thiazol-2-yl-phenyl)-acryloyl]-benzoic acid

Ex-18A: 2,4-Dimethoxy-5-thiazol-2-yl-benzaldehyde was prepared from 2-bromothiazole in a similar manner as described in Ex-12A through Ex-12D. Off-white solid, 83% yield. ¹H-NMR (CDCl₃) δ 10.34 (s, 1H), 8.86 (s, 1H), 7.89 (d, J=3.6 Hz, 1H), 7.36 (d, J=3.6 Hz, 1H), 6.56 (s, 1H), 4.12 (s, 3H), 4.02 (s, 3H). HRMS m/z: calc. 249.0460, found 249.0461.

Ex-18B: The title compound was prepared by condensing 2,4-dimethoxy-5-thiazol-2-yl-benzaldehyde (Ex-18A) and 4-acetylbenzoic acid in a similar manner as described in Ex-3B. Yellow solid, mp>260° C., 65% yield. ¹H-NMR (DMSO-d₆) δ 13.33 (bs, 1H), 8.74 (s, 1H), 8.22 (d, J=8 Hz, 2H), 8.04-8.12 (m, 3H), 7.95 (d, J=2 Hz, 1H), 7.82 (d, J=16 Hz, 1H), 7.76 (d, J=3 Hz, 1H), 6.94 (s, 1H), 4.14 (s, 3H), 4.05 (s, 1H). HRMS m/z=calc. 396.0906, found 396.0903.

### Example 19

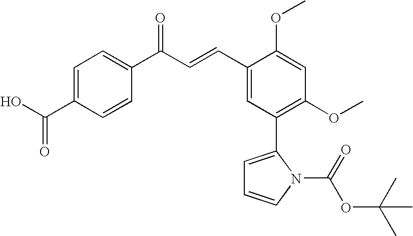

2-{5-[3-(4-Carboxy-phenyl)-3-oxo-E-propenyl]-2,4-dimethoxy-phenyl}-pyrrole-1-carboxylic acid tert-butyl ester

Ex-19A: 2-(5-Formyl-2,4-dimethoxy-phenyl)-pyrrole-1-carboxylic acid tert-butyl ester was prepared from pyrrole-1-carboxylic acid tert-butyl ester-2-boronic acid in a similar manner as described in Ex-3A, 81% yield. ¹H-NMR (CDCl₃) δ 10.32 (s, 1H), 7.76 (s, 1H), 7.31-7.33 (m, 1H), 6.43 (s, 1H), 6.22-6.24 (m, 1H), 6.14-6.16 (m, 1H), 3.98(s, 3H), 3.85 (s, 3H), 1.40 (s, 9H). HRMS (EI) Calcd. for C₁₈H₂₁NO₅: 331.1420. Found: 331.1421.

Ex-19B: The title compound was prepared by condensing 2-(5-formyl-2,4-dimethoxy-phenyl)-pyrrole-1-carboxylic acid tert-butyl ester (Ex-19A) and 4-acetylbenzoic acid in a similar manner as described in Ex-3B. Yellow solid, mp 205-207° C., 6% yield. ¹H-NMR (DMSO-d₆) δ 8.19 (d, J=5 Hz, 2H), 8.00-8.10 (m, 3H), 7.87 (s, 1H), 7.80 (d, J=16 Hz, 1H), 7.27-7.28(m, 1H), 6.71(s, 1H), 6.22-6.23 (m, 1H), 6.14-6.16 (m, 1H), 3.96 (s, 3H), 3.79(s, 3H), 1.29 (s, 9H). MS m/z=476 ([M-H]⁺). HMRS (EI) calcd. for C₂₇H₂₇NO₇: 477.1788; found: 477.1793.

### Example 20

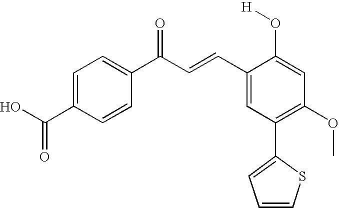

4-[3E-(2-Hydroxy-4-methoxy-5-thiophen-2-yl-phenyl)-acryloyl]-benzoic acid

Ex-20: 2-Hydroxy-4-methoxy-5-thiophen-2-yl-benzaldehyde (Ex-1B, 0.10 g, 0.43 mmol) and 4-acetylbenzoic acid (0.070 g, 0.43 mmol) were dissolved in a dimethylformamide-methanol solution (2.8 mL, 7:3). After complete dissolution, lithium methoxide (0.065 g, 1.7 mmol) was added and the resulting red slurry was stirred in the dark at room temperature for 18 h. The mixture was diluted with water (10 mL), acidified with a 1 N hydrochloric acid solution, and extracted with ethyl acetate (3×20 mL). The combined organic extracts were dried over sodium sulfate and evaporated to dryness. The crude oil was taken up in ethyl alcohol (5 mL) and warmed to 60° C. to obtain complete dissolution and allowed to cool to room temperature. Note: the compound appears to decompose with heating. The resulting precipitate was collected on filter paper and dried in vacuo to yield 0.025 g (15%) of the title compound as a dark yellow solid, mp 125° C. (dec). ¹H-NMR (300 MHz, DMSO-d₆) δ 10.73 (s, 1H), 8.18-8.22 (m, 3H), 8.09 (d, 2H, J=8.1 Hz), 8.05 (s, 1H), 7.87 (d, 1H, J=14.7 Hz), 7.60 (d, 1H, J=3.0 Hz), 7.49 (d, 1H, J=4.2 Hz), 7.11 (dd, 1H, J=4.2, 3.0 Hz), 6.67 (s, 1H), 3.90 (s, 3H). MS (ESI) m/z=−381 ([M+H]⁺, 100%). Anal. Calcd. for C₂₁H₁₆O₅S-EtOH: C, 64.77; H, 5.20; S, 7.52. Found: C, 64.68; H, 5.00; S, 7.77.

### Example 21

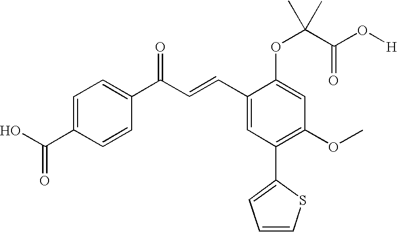

4-{3E-[2-(1-Carboxy-1-methyl-ethoxy)-4-methoxy-5-thiophen-2-yl-phenyl]-acryloyl}-benzoic acid

Ex-21A: 2-(2-Formyl-5-methoxy-4-thiophen-2-yl-phenoxy)-2-methyl-propionic acid ethyl ester was prepared in an analogous fashion as described in Ex-1C using ethyl 2-bromoisobutyrate. Silica gel chromatography (ethyl acetate/hexanes, 1:2) gave the expected product as a dark yellow solid (97%), mp 87-880C. ¹H-NMR (300 MHz, CDCl₃) δ 10.37 (s, 1H), 8.14 (s, 1H), 7.45 (dd, 1H, J=3.6, 1.2 Hz), 7.30 (d, 1H, J=5.4 Hz), 7.07 (dd, 1H, J=5.1, 3.6 Hz), 6.42 (s, 1H), 4.25 (q, 2H, J=6.9 Hz), 3.90 (s, 3H), 1.72 (s, 6H), 1.26 (t, 3H, J=6.9 Hz). MS (ESI) m/z=349 ([M+H]⁺, 100%). Anal. Calcd. for C₁₈H₂₀O₅S: C, 62.05; H, 5.79; S, 9.20. Found: C, 62.15; H, 5.82; S, 9.06.

Ex-21B: 2-(2-Formyl-5-methoxy-4-thiophen-2-yl-phenoxy)-2-methyl-propionic acid was prepared in an analogous fashion as described in Ex-2D. The crude solid was dried in vacuo to afford the product as a pale yellow solid (98%), mp 187-188° C. ¹H-NMR (300 MHz, CDCl₃) δ 9.33 (s, 1H), 7.99 (s, 1H), 7.47 (dd, 1H, J=3.6, 1.5 Hz), 7.37 (d, 1H, J=4.8 Hz), 7.11 (dd, 1H, J=4.8, 3.6 Hz), 6.67 (s, 1H), 4.00 (s, 3H), 1.75 (s, 6H). MS (ESI) m/z=321 ([M+H]⁺, 100%). Anal. Calcd. for C₁₆H₁₆O₅S: C, 59.99; H, 5.03; S, 10.01. Found: C, 59.80; H, 5.12; S, 9.87.

Ex-21C: 2-(2-Formyl-5-methoxy-4-thiophen-2-yl-phenoxy)-2-methyl-propionic acid (Ex-21B, 0.12 g, 0.39 mmol) and 4-acetylbenzoic acid (0.064 g, 0.39 mmol) were dissolved in a dimethylformamide-methanol solution (2.7 mL, 7:3). After complete dissolution, lithium methoxide (0.060 g, 1.6 mmol) was added and the resulting bright orange slurry was stirred in the dark at room temperature for 2 h. Upon completion, as determined by HPLC, the mixture was diluted with water (15 mL), acidified with a 1 N hydrochloric acid solution, and extracted with ethyl acetate (3×15 mL). The combined organic extracts were dried over sodium sulfate and evaporated to dryness. The crude oil was taken up in ethyl alcohol (5 mL) and warmed to 60° C. to obtain complete dissolution and allowed to cool to room temperature. The resulting precipitate was collected on filter paper and dried in vacuo to yield 0.15 g (85%) of the title compound as a dark yellow solid, mp 223-225-C. ¹H-NMR (300 MHz, DMSO-d₆) δ 8.31 (s, 1H), 8.23 (d, 2H, J=8.1 Hz), 8.10 (d, 2H, J=8.1 Hz), 8.06 (s, 1H), 7.95 (d, 1H, J=16.2 Hz), 7.69 (d, 1H, J=3.0 Hz), 7.55 (d, 1H, J=5.1 Hz), 7.14 (dd, 1H, J=5.1, 3.0 Hz), 6.58 (s, 1H), 3.88 (s, 3H), 1.66 (s, 6H). MS (ESI) m/z=467 ([M+H]⁺, 100%). Anal. Calcd. for C₂₅H₂₂O₇S.⅓H₂O: C, 63.55; H, 4.84; S, 6.79. Found: C, 63.39; H, 5.02; S, 6.53.

### Example 22

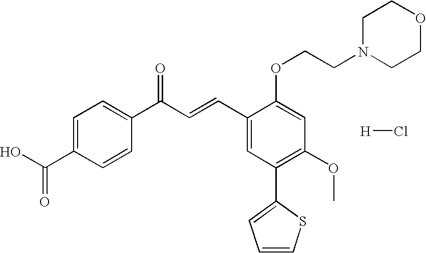

4-{3E-[4-Methoxy-2-(2-morpholin-4-yl-ethoxy)-5-thiophen-2-yl-phenyl]-acryloyl}-benzoic acid, hydrochloride

Ex-22A: 4-Methoxy-2-(2-morpholin-4-yl-ethoxy)-5-thiophen-2-yl-benzaldehyde was prepared in an analogous fashion as described in Ex-1C using 4-(2-chloroethyl)morpholine. Silica gel chromatography (80 to 100% ethyl acetate/hexanes then 5% methanol/methylene chloride) gave of the expected product as a off-white solid (81%). ¹H-NMR (300 MHz, CDCl₃) δ 10.36 (s, 1H), 8.12 (s, 1H), 7.44 (dd, 1H, J=3.6, 1.5 Hz), 7.30 (dd, 1H, J=5.1, 1.5 Hz), 7.07 (dd, 1H, J=5.1, 3.6 Hz), 6.53 (s, 1H), 4.27 (t, 2H, J=6.3 Hz), 4.00 (s, 3H), 3.72-3.76 (m, 4H), 2.89 (t, 2H, J=6.3 Hz), 2.60-2.63 (m, 4H). MS (ESI) m/z=348 ([M+H]⁺, 100%). HRMS (EI) Calcd. for C₁₈H₂₁NO₄S: 347.1191. Found: 347.1188.

Ex-22B: 4-Methoxy-2-(2-morpholin-4-yl-ethoxy)-5-thiophen-2-yl-benzaldehyde (Ex-22A, 0.15 g, 0.43 mmol) and 4-acetylbenzoic acid (0.071 g, 0.43 mmol) were dissolved in a dimethylformamide-methanol solution (3.0 mL, 7:3). After complete dissolution, lithium methoxide (0.065 g, 1.7 mmol) was added and the resulting bright orange slurry was stirred in the dark at room temperature for 2 h. Upon completion, as determined by HPLC, the mixture was diluted with water (10 mL), acidified with a 1 N hydrochloric acid solution, and extracted with an ethyl acetate:tetrahydrofuran mixture (1:1, 6×20 mL). The combined organic extracts were dried over sodium sulfate and evaporated to dryness. The crude solid was slurried in ethyl alcohol (5 mL) to remove residual impurities and the resulting solid was collected on filter paper and dried in vacuo to yield 0.21 g (98%) of the title compound as a dark yellow solid, mp: 255° C. (dec). ¹H-NMR (300 MHz, DMSO-d₆) δ 8.34 (s, 1H), 8.26 (d, 2H, J=8.7 Hz), 8.11 (d, 2H, J=8.7 Hz), 8.08 (s, 1H), 7.95 (d, 1H, J=15.9 Hz), 7.71 (d, 1H, J=3.3 Hz), 7.55 (d, 1H, J=4.5 Hz), 7.15 (dd, 1H, J=4.5, 3.3 Hz), 6.94 (s, 1H), 4.68 (brs, 2H), 4.04 (s, 3H), 3.98 (brs, 2H), 3.81-3.88 (brm, 2H), 3.70 (brs, 2H), 3.54-3.58 (brm, 2H), 3.29 (brs, 2H). MS (ESI) m/z=494 ([M+H]⁺, 100%). Anal. Calcd. for C₂₇H₂₈ClNO₆S: C, 61.18; H, 5.32; Cl, 6.69; N, 2.64; S, 6.05. Found: C, 61.18; H, 5.41; Cl, 6.16; N, 2.73; S, 5.87.

### Example 23

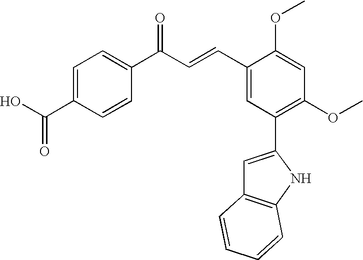

4-{3E-[5-(1H-Indol-2-yl)-2,4-dimethoxy-phenyl]-aciryloyl}-benzoic acid

Ex-23A: 2-(5-Formyl-2,4-dimethoxy-phenyl)-indole-1-carboxylic acid tert-butyl ester (2.0g, 5.2 mmol) was dissolved in 100 ml of THF, and Bu₄NF (6.86g, 26 mmol) was added. The reaction mixture was stirred at room temperature overnight. No reaction occured at this condition. Then, Bu₄NF (6.86g, 26 mmol) was added to the mixture, and the mixture was stirred at reflux for 4 days. The reaction was about 50% completion (HPLC). The reaction mixture was poured into CH₂Cl₂, and washed with water and brine. The organic phase was dried over MgSO₄, and concentrated. The residue was purified by column chromatography (EtOAc: Hex, 2:1) to give 0.45 g (30%) of 5-(1H-indol-2-yl)-2,4-dimethoxy-benzaldehyde. ¹H-NMR (CDCl₃) δ 10.37 (s, 1H), 9.25 (br, 1H), 8.28 (s, 1H), 7.63(d, J=8 Hz, 1H), 7.39 (d, J=8 Hz, 1H), 7.08-7.20 (m, 2H), 6.92(d, J=2 Hz, 1H), 6.56 (s, 1H) 4.11 (s, 3H), 4.00 (s, 3H). HMRS (EI) calcd. for C₁₇H₁₅NO₃: 281.1052; found: 281.1049.

Ex-23B: The title compound was prepared by condensing 5-(1H-indol-2-yl)-2,4-dimethoxy-benzaldehyde (Ex-23A) and 4-acetylbenzoic acid in a similar manner as described in Ex-3B. Red solid, mp 210-212° C., 66% yield. ¹H-NMR (Aceton-d₆) δ 10.53 (br, s, 1H), 8.32 (s, 1H), 8.14-8.21 (m, 5H), 7.89 (d, J=15 Hz, 1H), 7.52 (d, J=8 Hz, 1H), 7.38 (d, J=7 Hz, 1H), 6.97-7.07(m, 3H), 6.87(s, 1H), 4.07 (s, 3H), 4.02(s, 3H), MS m/z=427 ([M]+). HMRS (EI) calcd. for C₂₆H₂₁NO₅: 427.1420; found: 427.1435.

### Example 24

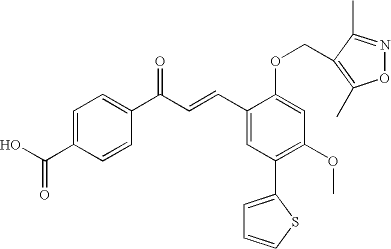

4-{3E-[2-(3,5-Dimethyl-isoxazol-4-ylmethoxy)-4-methoxy-5-thiophen-2-yl-phenyl]-acryloyl}-benzoic acid

Ex-24A: 2-(3,5-Dimethyl-isoxazol-4-ylmethoxy)-4-methoxy-5-thiophen-2-yl-benzaldehyde was prepared in a similar manner as described in Ex-1C using 4-chloromethyl-3,5-dimethyl-isoxazole. ¹H-NMR (CDCl₃) δ 10.26 (s, 1H), 8.14 (s, 1H), 7.45 (d, J=6 Hz, 1H), 7.32 (d, J=5 Hz, 1H), 7.07-710 (m, 1H), 6.58 (s, 1H), 4.96 (s, 2H), 4.04 (s, 3H), 2.46 (s, 3H), 2.32 (s, 3H).

Ex-24B: The title compound was prepared by condensing 2-(3,5-dimethyl-isoxazol-4-ylmethoxy)-4-methoxy-5-thiophen-2-yl-benzaldehyde (Ex-24A) and 4-acetylbenzoic acid in a similar manner as described in Ex-3B. Yellow solid, mp 213-215° C. ¹H-NMR (CDCl₃) δ 8.20 (d, J=9 Hz, 2H), 7.88-8.03 (m, 4H), 7.58 (d, J=16 Hz, 1H), 7.44 (d, J=4 Hz, 1H), 7.34(d, J=5 Hz, 1H), 7.12(dd, J=4, 5 Hz, 1H), 6.63 (s, 1H), 4.97(s, 2H), 4.01 (s, 3H), 2.46(s, 3H), 2.34 (s, 3H). MS m/z=490 ([M+H]⁺). HRMS (ES+) Calcd. for C₂₇H₂₂NO₆S: 490.1324. Found: 490.1321.

### Example 25

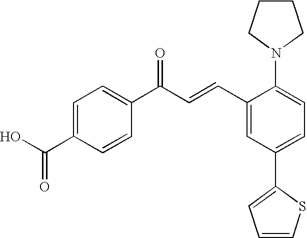

4-[3E-(2-Pyrrolidin-1-yl-5-thiophen-2-yl-phenyl)-acryloyl]-benzoic acid

Ex-25A: A solution of 2-fluoro-5-thiophen-2-yl-benzaldehyde (1.42g, 6.89 mmol) in pyrrolidine was refluxed (10 mL). After 4.5 days the reaction mixture was cooled and diluted with ethyl acetate. The solution of ethyl acetate was washed with hydrochloric acid (0.5M) sodium carbonate (2M) and saturated solution of sodium bicarbonate, dried over sodium sulfate, and concentrated. The crude product was purified by flash chromatography. Elution with ethyl acetate (20%, v/v, in hexane) afforded 2-pyrrolidin-1-yl-5-thiophen-2-yl-benzaldehyde (0.5g, 32%). ¹H NMR (CDCl₃) δ 10.14 (s, 1H), 7.94 (d, J=2 Hz, 1H), 7.62 (dd, J=2.7, 9 Hz, 1H), 7.22-7.20 (m, 2H), 7.07-7.04 (m, 1H), 6.86 (d, J=9 Hz, 1H), 3.41 (m, 4H), 2.01 (m, 4H).

Ex-25B: The title compound was prepared by condensing 2-pyrrolidin-1-yl-5-thiophen-2-yl-benzaldehyde (Ex-25A) and 4-acetylbenzoic acid in a similar manner as described in Ex-3B. Red solid, mp 208-209° C. ¹H-NMR (DMSO-d₆) δ 12.50 (bs, 1H), 8.22 (d, J=8.5 Hz, 2H), 8.09-7.99 (m, 4H), 7.73 (d, J=15.5 Hz, 1H), 7.52-7.41 (m, 3H), 7.10-7.07 (m, 1H), 6.93 (d, J=9.0 Hz, 1H), 3.28 (m, 4H), 1.87 (m, 4H).

### Example 26

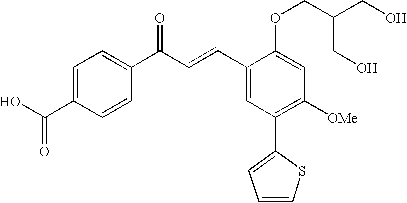

4-{3E-[2-(3-Hydroxy-2-hydroxymethyl-propoxy)-4-methoxy-5-thiophen-2-yl-phenyl]-acryloyl}-benzoic acid

Ex-26A: To a solution of 2-hydroxy-4-methoxy-5-thiophen-2-yl-benzaldehyde (10.0 g, 42.7 mmol) in N,N-dimethylformamide (100 mL) was added potassium carbonate (11.8 g, 85.4 mmol) and the resulting yellow slurry was heated to 80° C. Once at 80° C., methanesulfonic acid 3-(tert-butyl-dimethyl-silanyloxy)-2-(tert-butyl-dimethyl-silanyloxymethyl)-propyl ester (Ex-14A, 19.5 g, 46.9 mmol) was added dropwise and the reaction was stirred for an additional 24 h at 80° C. and cooled to room temperature. The mixture was diluted with water (500 mL) and extracted with ethyl acetate (3×150 mL). The combined organic layers was sequentially washed with a saturated sodium bicarbonate solution (1×150 mL), water (1×150 mL), and brine (1×150 mL), dried over sodium sulfate, and concentrated to a brown oil. Silica gel chromatography (100% ethyl acetate to 10% ethyl acetate/hexanes) gave 19.0 g (81%) of 2-[3-(tert-butyl-dimethyl-silanyloxy)-2-(tert-butyl-dimethyl-silanyloxymethyl)-propoxy]-4-methoxy-5-thiophen-2-yl-benzaldehyde as an off-white solid, mp 91-92° C. ¹H-NMR (300 MHz, CDCl₃) δ 10.37 (s, 1H), 8.12 (s, 1H), 7.44 (dd, 1H, J=3.6, 1.2 Hz), 7.29 (d, 1H, J=5.1 Hz), 7.07 (dd, 1H, J=5.1, 3.6 Hz), 6.54 (s, 1H), 4.19 (d, 2H, J=6.0 Hz), 3.99 (s, 3H), 3.72-3.82 (m, 4H), 2.28 (pentet, 1H, J=6.0 Hz), 0.88 (s, 18H), 0.048 (s, 12H). MS (EI) m/z=550 ([M]+, 100%). Anal. Calcd. for C₂₈H₄₆O₅SSi₂: C, 61.05; H, 8.42; S, 5.82. Found: C, 61.20; H, 8.74; S, 5.69.

Ex-26B: 2-(3-Hydroxy-2-hydroxymethyl-propoxy)-4-methoxy-5-thiophen-2-yl-benzaldehyde was prepared in an analogous fashion as described in Ex-14C. Silica gel chromatography (ethyl acetate/hexanes, 1:9) gave the expected product as an off-white solid. ¹H-NMR (300 MHz, CDCl₃) δ 10.17 (s, 1H), 8.03 (s, 1H), 7.43 (dd, 1H, J=3.6, 1.2 Hz), 7.31 (d, 1H, J=5.1 Hz), 7.08 (dd, 1H, J=5.1, 3.6 Hz), 6.58 (s, 1H), 4.32 (d, 2H, J=6.0 Hz), 4.01 (s, 3H), 3.95-3.99 (m, 4H), 2.51 (t, 2H, J=5.1 Hz), 2.33 (pentet, 1H, J=5.4 Hz). MS (EI) m/z=322 ([M]+, 100%). HRMS (EI) Calcd. for C₁₆H₁₈O₅S: 322.0875. Found: 322.0873.

Ex-26C: The title compound was prepared by condensing 2-(3-hydroxy-2-hydroxymethyl-propoxy)-4-methoxy-5-thiophen-2-yl-benzaldehyde (Ex-26B) and 4-acetylbenzoic acid in a similar manner as described in Ex-3B. Light orange solid, mp 219-220° C., 61% yield. ¹H-NMR (300 MHz, DMSO-d₆) δ 8.36 (s, 1H), 8.20 (d, 2H, J=7.5 Hz), 8.05-8.11 (m, 3H), 7.93 (d, 1H, J=16.2 Hz), 7.67 (d, 1H, J=3.0 Hz), 7.52 (d, 1H, J=5.1 Hz), 7.13 (dd, 1H, J=5.1, 3.0 Hz), 6.88 (s, 1H), 4.66 (brs, 2H), 4.23 (d, 2H, J=6.3 Hz), 4.01 (s, 3H), 3.55-3.66 (m, 4H), 2.09-2.14 (m, 1H). MS (ESI) m/z 10=469 ([M+H]⁺, 100%). Anal. Calcd. for C₂₅H₂₄O₇S—H₂O: C, 61.72; H, 5.39; S, 6.59.

Found: C, 61.93; H, 5.30; S, 7.06.

### Example 27

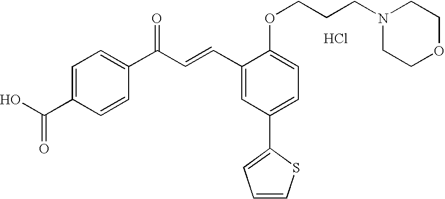

4-{3E-[2-(3-Morpholin-4-yl-propoxy)-5-thiophen-2-yl-phenyl]-acryloyl}-benzoic acid, hydrochloride

Ex-27A: 2-(3-Morpholin-4-yl-propoxy)-5-thiophen-2-yl-benzaldehyde was prepared in a similar manner as described in Ex-22A, 80% yield. ¹H-NMR (DMSO-D₆) δ 10.36 (s, 1H), 7.90 (dd, J=3, 5 Hz, 1H), 7.82 (d, 1H), 7.48 (d, 1H), 7.44 (d, 1H), 7.25 (d, 1H), 7.09 (t, 1H), 4.18 (t, 2H), 3.53 (m, 4H), 3.28 (br s, 2H), 2.43 (m, 4H), 1.89 (q, 2H).

Ex-27B: The title compound was prepared by condensing 2-(3-morpholin-4-yl-propoxy)-5-thiophen-2-yl-benzaldehyde (Ex-27A) and 4-acetylbenzoic acid in a similar manner as described in Ex-3B. Yellow solid, 67% yield, mp 234-236° C. ¹H-NMR (DMSO-d₆) δ 13.32 (br s, 1H), 11.10 (br s, 1H), 8.21 (m, 3H), 8.02 (m, 3H), 7.67 (dd, J=2, 2 Hz, 1H), 7.56 (d, 1H), 7.50 (d, 1H), 7.14 (m, 2H), 4.21(t, 2H), 3.86 (m, 4H), 3.23 (m, 6H), 2.29 (q, 2H). MS m/z=478 ([M+H]⁺, 100%). Anal. calculated for C₂₇H₂₈ClNO₅S.3/2H₂O: C, 59.94, H, 5.78, S, 5.93; found C: 60.20, H: 5.65, S: 5.94

### Example 28

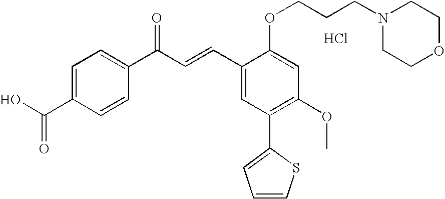

4-{3E-[4-Methoxy-2-(3-morpholin-4-yl-propoxy)-5-thiophen-2-yl-phenyl]-acryloyl}-benzoic acid, hydrochloride

Ex-28A: 4-Methoxy-2-(3-morpholin-4-yl-propoxy)-5-thiophen-2-yl-benzaldehyde was prepared in a similar manner as described in Ex-22A, 78% yield. ¹H-NMR (DMSO-D₆) δ 10.21 (s, 1H), 7.88 (s, 1H), 7.46 (m, 2H), 7.06 (t, 1H), 6.82 (s, 1H), 4.24 (t, 2H), 4.00 (s, 3H), 3.53 (m, 4H), 3.28 (m, 2H), 2.34 (m, 4H), 1.93 (q, 2H).

Ex-28B: The title compound was prepared by condensing 4-methoxy-2-(3-morpholin-4-yl-propoxy)-5-thiophen-2-yl-benzaldehyde (Ex-28A) and 4-acetylbenzoic acid in a similar manner as described in Ex-3B. Yellow solid, 72% yield, mp 188-191° C. (dec). ¹H-NMR (DMSO-d₆) δ 12.63 (br s, 1H), 11.08 (br s, 1H), 8.33 (s, 1H), 8.22 (d, 2H), 8.05 (m, 3H), 7.89 (d, 1H), 7.65 (d, 1H), 7.49 (d, 1H), 7.10 (t, 1H), 6.84 (s, 1H), 4.30 (t, 2H), 3.98 (s, 3H), 3.84 (m, 4H), 3.21 (m, 6H), 2.28 (q, 2H). MS m/z=508 ([M+H]⁺, 100%). Anal. calculated for C₂₈H₃₂ClNO₇S. H₂O: C, 59.83, H, 5.74, S, 5.70; found C: 59.69, H: 5.80, S: 5.55.

### Example 29

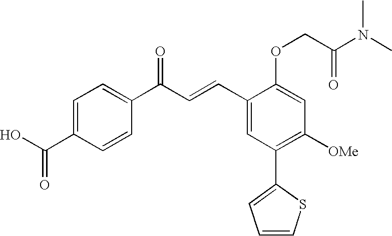

4-[3E-(2-Dimethylcarbamoylmethoxy-4-methoxy-5-thiophen-2-yl-phenyl)-acryloyl]-benzoic acid

Ex-29A: 2-(2-Formyl-5-methoxy-4-thiophen-2-yl-phenoxy)-N,N-dimethyl-acetamide was prepared in an analogous fashion as described in Ex-1C using 2-chloro-N,N-dimethylacetamide. Methylene chloride was used in place of ethyl acetate for the work up procedure. The crude solid was slurried in ethyl acetate (25 mL) to remove residual impurities. The resulting solid was collected on filter paper and dried in vacuo to give the expected product as a pale yellow solid (85%), mp 197-1980C. ¹H-NMR (300 MHz, CDCl₃) δ 10.38 (s, 1H), 8.13 (s, 1H), 7.44 (d, 1H, J=3.6 Hz), 7.30 (dd, 1H, J=5.1, 1.8 Hz), 7.07 (dd, 1H, J=5.1, 3.6 Hz), 6.73 (s, 1H), 4.89 (s, 2H), 3.99 (s, 3H), 3.15 (s, 3H), 2.99 (s, 3H). MS (EI) m/z=319 ([M]+, 100%). Anal. Calcd. for C₁₆H₁₇NO₄S.⅕H₂O: C, 59.50; H, 5.43; N, 4.34; S, 9.93. Found: C, 59.65; H, 5.42; N, 4.40; S, 9.69.

Ex-29B: The title compound was prepared by condensing 2-(2-formyl-5-methoxy-4-thiophen-2-yl-phenoxy)-N,N-dimethyl-acetamide (Ex-29A) and 4-acetylbenzoic acid in a similar manner as described in Ex-3B. Yellow solid, mp 228-229° C., 75% yield. ¹H-NMR (300 MHz, DMSO-d₆) δ 8.31 (d, 2H, J=9.3 Hz), 8.22 (d, 2H, J=13.3 Hz), 8.08 (d, 2H, J=9.3 Hz), 7.95 (s, 1H), 7.65 (d, 1H, J=2.7 Hz), 7.52 (d, 1H, J=5.1 Hz), 7.13 (dd, 1H, J=5.1, 2.7 Hz), 6.85 (s, 1H), 5.11 (s, 2H), 3.99 (s, 3H), 3.06 (s, 3H), 2.93 (s, 3H). MS (EI) m/z=465 ([M]+, 100%). HRMS (EI) Calcd. for C₂₅H₂₃NO₆S: 465.1246. Found: 465.1246.

### Example 30

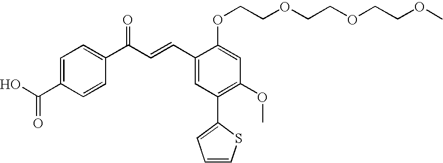

4-[3E-(4-Methoxy-2-{2-[2-(2-methoxy-ethoxy)-ethoxy]-ethoxy)-5-thiophen-2-yl-phenyl)-acryloyl]-benzoic acid

Ex-30A: Methanesulfonic acid 2-[2-(2-methoxy-ethoxy)-ethoxy]-ethyl ester was prepared in an analogous fashion as described in Ex-14A using di(ethylene glycol) methyl ether. The crude orange oil was dried in vacuo to give the expected product (oil) and was used without any further purification (99%). ¹H-NMR (300 MHz, CDCl₃) δ 4.37-4.40 (m, 2H), 3.76-3.78 (m, 2H), 3.61-3.70 (m, 6H), 3.53-3.57 (d, 2H), 3.38 (s, 3H), 3.08 (s, 3H). MS (ESI) m/z=243 ([M+H]⁺, 100%). HRMS (ESI) Calcd. for C₈H₁₈O₆S: 243.0902. Found: 243.0914.

Ex-30B: 4-Methoxy-2-{2-[2-(2-methoxy-ethoxy)-ethoxy]-ethoxy}-5-thiophen-2-yl-benzaldehyde was prepared in an analogous fashion as as described in Ex-1C using methanesulfonic acid 2-[2-(2-methoxy-ethoxy)-ethoxy]-ethyl ester (Ex-30A). Silica gel chromatography (ethyl acetate/hexanes, 8:1) gave the expected product as a pale yellow oil (70%). ¹H-NMR (300 MHz, CDCl₃) δ 10.38 (s, 1H), 8.12 (s, 1H), 7.44 (d, 1H, J=3.6 Hz), 7.30 (d, 1H, J=5.4 Hz), 7.07 (dd, 1H, J=5.4, 3.6 Hz), 6.57 (s, 1H), 4.31 (t, 2H, J=4.8 Hz), 3.99 (s, 3H), 3.94 (t, 2H, J=4.8 Hz), 3.74-3.78 (m, 2H), 3.62-3.69 (m, 4H), 3.53-3.56 (m, 2H), 3.37 (s, 3H). MS (EI) m/z=380 ([M]⁺, 100%). HRMS (ESI) Calcd. for C₈H₁₈O₆S: 243.0902. Found: 243.0914.

Ex-30C: The title compound was prepared by condensing 4-methoxy-2-{2-[2-(2-methoxy-ethoxy)-ethoxy]-ethoxy}-5-thiophen-2-yl-benzaldehyde (Ex-30B) and 4-acetylbenzoic acid in a similar manner as described in Ex-3B. Yellow solid, mp 137-138° C., 82% yield. ¹H-NMR (300 MHz, DMSO-d₆) δ 8.20-8.23 (m, 3H), 8.09 (d, 2H, J=8.3 Hz), 8.01 (m, 2H), 7.66 (d, 1H, J=3.6 Hz), 7.52 (d, 1H, J=5.1 Hz), 7.13 (dd, 1H, J=5.1, 3.6 Hz), 6.88 (s, 1H), 4.37 (t, 2H, J=3.6 Hz), 4.01 (s, 3H), 3.89 (t, 2H, J=3.6 Hz), 3.64-3.67 (m, 2H), 3.53-3.56 (m, 2H), 3.47-3.50 (m, 2H), 3.36-3.95 (m, 2H), 3.19 (s, 3H). MS (ESI) m/z=527 ([M+H]⁺, 100%). Anal. Calcd. for C₂₈H₃₀O₈S: C, 63.86; H, 5.74; S, 6.09. Found: C, 64.08; H, 5.77; S, 6.09.

### Example 31

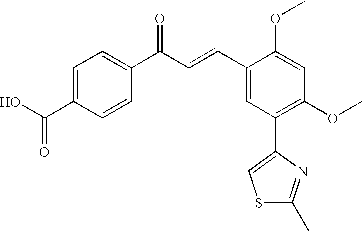

4-{3E-[2,4-Dimethoxy-5-(2-methyl-thiazol-4-yl)-phenyl]-acryloyl}-benzoic acid

Ex-31A: A solution of 2-bromo-1-(3,4-dimethoxy-phenyl)-ethanone (0.62g, 2.39 mmol) and thioacetamide (0.18g, 2.39 mmol) in ethanol (30 mL) was refluxed for 2 hours and the solvent was removed under reduced pressure. The product, 4-(3,4-dimethoxy-phenyl)-2-methyl-thiazole (0.56g, 100%) was obtained as a white solid and used without further purification. To a suspension of 4-(3,4-dimethoxy-phenyl)-2-methyl-thiazole obtained above (0.70g, 2.97 mmol) in dichloromethane (60 mL) at 0° C. was added dichloromethyl methyl ether (0.40 mL, 4.46 mmol) followed by addition of titanium tetrachloride (1.0 M solution in dichloromethane, 8.9 mL, 8.9 mmol) dropwise. The reaction mixture was allowed to stir overnight at ambient temperature and then poured into ice. The aqueous solution was extracted with dichloromethane. The solution of dichloromethane was washed with hydrochloric acid (0.5M), saturated solution of sodium bicarbonate and brine, dried over sodium sulfate and concentrated. The product, 2,4-dimethoxy-5-(2-methyl-thiazol-4-yl)-benzaldehyde, was obtained as a white solid. ¹H NMR (CDCl₃) δ 10.33 (s, 1H), 8.67 (s, 1H), 7.56 (s, 1H), 6.52 (s, 1H), 4.03 (s, 3H), 3.99 (s, 3H), 2.75 (s, 3H).

Ex-31B: The title compound was prepared by condensing 2,4-dimethoxy-5-(2-methyl-thiazol-4-yl)-benzaldehyde (Ex-31A) and 4-acetylbenzoic acid in a similar manner as described in Ex-3B. Yellow solid, mp 201-202° C. (dec.). ¹H-NMR (DMSO-d₆) δ 8.47 (s, 1H), 8.14-7.97 (m, 5H), 7.76 (s, 1H), 7.65 (d, J=15.8 Hz, 1H), 6.81 (s, 1H), 4.00 (s, 3H), 3.98 (s, 3H), 2.69 (s, 3H). MS m/z=409 (M+, 70%), 378 ([M−OCH₃]⁺, 100%).

### Example 32

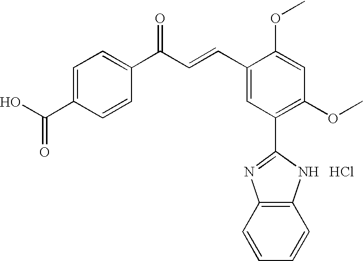

4-13E-[5-(1H-Benzoimidazol-2-yl)-2,4-dimethoxy-phenyl]-acryloyl)-benzoic acid

Ex-32A: A solution of benzene-1,2-diamine (2.60g, 24.1 mmol) and 2,4-dimethoxy-benzaldehyde (4.0g, 24.1 mmol) in ethanol (60 mL) containing catalytic amount of acetic acid was refluxed overnight. Solvent was then evaporated under reduced pressure. The residue oil was triturated in ethyl acetate to obtain 2-(2,4-dimethoxyphenyl)-1H-benzoimidazole (0.76g, 12%). The crude product was used without further purification. To a solution of 2-(2,4-dimethoxy-phenyl)-1H-benzoimidazole obtained above (0.76g, 2.99 mmol) in dichloromethane (20 mL) was added dichloromethyl methyl ether (0.41 mL, 4.48 mmol) followed by addition of titanium tetrachloride (11.0M in dichloromethane, 9.0 mL, 9.0 mmol) at 0° C. The reaction mixture was allowed to stir overnight at ambient temperature and then poured into ice. A solution of sodium hydroxide (5M) was added dropwise until the pH of the solution was about 12. The basic solution was extracted with dichloromethane. The combined solution of dichloromethane was subsequently washed with brine, dried over sodium carbonate and concentrated. The product, 5-(1H-benzoimidazol-2-yl)-2,4-dimethoxybenzaldehyde (0.40g, 47%), was obtain and used without further purification. ¹H NMR (CDCl₃) δ 10.32 (s, 1H), 10.27 (bs, 1H), 9.03 (s, 1H), 7.83 (d, J=9 Hz, 1H), 7.48-7.45 (m, 1H), 7.31-7.22 (m, 1H), 6.58 (s, 1H), 4.18 (s, 3H), 4.01 (s, 3H). MS m/z=282 (M+, 100%).

Ex-32B: The title compound was prepared by condensing 5-(1H-benzoimidazol-2-yl)-2,4-dimethoxy-benzaldehyde (Ex-32A) and 4-acetylbenzoic acid in a similar manner as described in Ex-3B. Yellow solid, mp>240° C. (dec.). ¹H-NMR (DMSO-d₆) δ 8.72 (s, 1H), 12.10 (s, 1H), 8.18 (d, J=8.4 Hz, 2H), 8.08-8.02 (m, 3H), 7.80 (d, J=15.4 Hz, 1H), 7.59 (s, 2H), 7.17-7.13 (m, 2H), 6.89 (s, 1H), 4.10 (s, 3H), 4.03 (s, 3H). MS m/z=429 ([M+H]⁺, 100%).

### Example 33

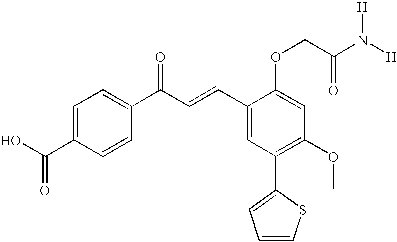

4-[3E-(2-Carbamoylmethoxy-4-methoxy-5-thiophen-2-yl-phenyl)-acryloyl]-benzoic acid

Ex-33A: 2-(2-Formyl-5-methoxy-4-thiophen-2-yl-phenoxy)-acetamide was prepared in an analogous fashion as described in Ex-1C using 2-bromoacetamide. Silica gel chromatography (ethyl acetate/hexanes, 8:1) gave the expected product as a pale yellow solid (75%), mp: 178-179° C. ¹H-NMR (300 MHz, CDCl₃) δ 10.05 (s, 1H), 7.99 (s, 1H), 7.67 (brs, 1H), 7.44 (d, 1H, J=3.6 Hz), 7.34 (d, 1H, J=5.4 Hz), 7.10 (dd, 1H, J=5.4, 3.6 Hz), 6.48 (s, 1H), 5.67 (brs, 1H), 4.64 (s, 2H), 4.02 (s, 3H). MS (EI) m/z=291 ([M]+, 100%). Anal. Calcd. for C₁₄H₁₃NO₄S: C, 57.72; H, 4.50; N, 4.81; S, 11.01. Found: C, 57.63; H, 4.50; N, 4.87; S, 11.03.

Ex-33B: The title compound was prepared by condensing 2-(2-formyl-5-methoxy-4-thiophen-2-yl-phenoxy)-acetamide (Ex-33A) and 4-acetylbenzoic acid in a similar manner as described in Ex-3B. Yellow solid, 70% yield, mp 235° C. (dec.). ¹H-NMR (300 MHz, DMSO-d₆) δ 8.26-8.30 (m, 3H), 8.08-8.11 (m, 4H), 7.67 (d, 1H, J=2.7 Hz), 7.65 (brs, 1H), 7.53 (d, 1H, J=4.0 Hz), 7.49 (brs, 1H), 7.13 (m, 1H), 6.77 (s, 1H), 4.75 (s, 2H), 3.97 (s, 3H). MS (EI) m/z=437 ([M]⁺, 100%). HRMS (EI) Calcd. for C₂₃H₁₉NO₆S: 437.0933. Found: 437.0924.

### Example 34

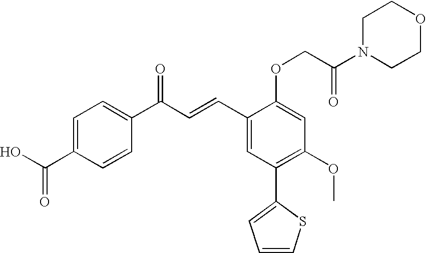

4-{3E-[4-Methoxy-2-(2-morpholin-4-yl-2-oxo-ethoxy)-5-thiophen-2-yl-phenyl]-acryloyl}-benzoic acid

Ex-34A: 4-Methoxy-2-(2-morpholin-4-yl-2-oxo-ethoxy)-5-thiophen-2-yl-benzaldehyde was prepared in an analogous fashion as described in Ex-1C using 4-(2-chloroacetyl)morpholine. Silica gel chromatography (80% ethyl acetate/hexanes to 100% ethyl acetate) gave the expected product as a pale yellow solid, mp 200-201° C. ¹H-NMR (300 MHz, CDCl₃) δ 10.33 (s, 1H), 8.12 (s, 1H), 7.44 (d, 1H, J=3.6 Hz), 7.31 (d, 1H, J=5.1 Hz), 7.08 (dd, 1H, J=5.1, 3.6 Hz), 6.74 (s, 1H), 4.89 (s, 2H), 4.00 (s, 3H), 3.67 (brs, 8H). MS (ESI) m/z=362 ([M+H]⁺, 100%). Anal. Calcd. for C₁₈H₁₉NO₅S: C, 59.82; H, 5.30; N, 3.88; S, 8.87. Found: C, 59.88; H, 5.36; N, 3.90; S, 8.75.

Ex-34B: The title compound was prepared by condensing 4-methoxy-2-(2-morpholin-4-yl-2-oxo-ethoxy)-5-thiophen-2-yl-benzaldehyde (Ex-34A) and 4-acetylbenzoic acid in a similar manner as described in Ex-3B. Orange solid, mp 231-233° C., 70% yield. ¹H-NMR (300 MHz, DMSO-d₆) δ 8.28-8.35 (m, 3H), 8.21 (s, 1H), 8.07-8.11 (m, 3H), 7.66 (d, 1H, J=3.3 Hz), 7.52 (d, 1H, J=5.1 Hz), 7.13 (dd, 1H, J=5.1, 3.3 Hz), 6.87 (s, 1H), 5.13 (s, 2H), 4.00 (s, 3H), 3.65 (brm, 4H), 3.54-3.55 (m, 4H). MS (EI) m/z=507 ([M]⁺, 100%). Anal. Calcd. for C₂₇H₂₅NO₇S.½EtOH: C, 63.55; H, 5.61; N, 2.60; S, 5.95. Found: C, 63.13; H, 5.55; N, 2.53; S, 5.84.

### Example 35

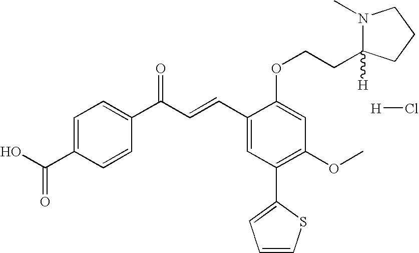

4-(3E-{4-Methoxy-2-[2-(1-methyl-pyrrolidin-2-yl)-ethoxy]-5-thiophen-2-yl-phenyl}-acryloyl)-benzoic acid, hydrochloride

Ex-35A: Methanesulfonic acid 2-(1-methyl-pyrrolidin-2-yl)-ethyl ester was prepared in an analogous fashion as described in Ex-14A using (S)-(−)-1-methyl-2-pyrrolidinemethanol. The crude orange oil was dried in vacuo to give the expected product and was used without any further purification (40%). ¹H-NMR (300 MHz, CDCl₃) δ 4.99-5.04 (m, 1H), 4.41-4.51 (m, 1H), 4.19-4.29 (m, 1H), 3.88-3.94 (m, 1H), 3.49 (s, 3H), 3.17-3.29 (m, 1H), 2.95-3.05 (m, 1H), 2.74 (s, 3H), 2.41-2.58 (m, 3H), 1.98-2.08 (m, 2H). MS (EI) m/z=207 ([M]+, 100%). HRMS (EI) Calcd. for C₁₈H₁₉NO₅S: 207.0929. Found: 207.0922.

Ex-35B: 4-Methoxy-2-[2-(1-methyl-pyrrolidin-2-yl)-ethoxy]-5-thiophen-2-yl-benzaldehyde was prepared in an analogous fashion as described in Ex-1C using Methanesulfonic acid 2-(1-methyl-pyrrolidin-2-yl)-ethyl ester (Ex-35A). Silica gel chromatography (10% methanol/methylene chloride to 15% methanol/methylene chloride) gave 0.50 g (70%) of the expected product as a pale yellow oil. ¹H-NMR (300 MHz, CDCl₃, major isomer) δ 10.35 (s, 1H), 8.09 (s, 1H), 7.42-7.44 (m, 1H), 7.30 (d, 1H, J=5.1 Hz), 7.06-7.09 (m, 1H), 6.49 (s, 1H), 4.80 (m, 1H), 4.20-4.26 (m, 1H), 3.98 (s, 3H), 2.64-2.84 (m, 2H), 2.47 (s, 3H), 1.80-2.33 (m, 7H). MS (EI) m/z=345 ([M]+, 100%). HRMS (EI) Calcd. for C₁₈H₁₉NO₅S: 345.1399. Found: 345.1401.

Ex-35C: The title compound was prepared by condensing 4-methoxy-2-[2-(1-methylpyrrolidin-2-yl)-ethoxy]-5-thiophen-2-yl-benzaldehyde (Ex-35B) and 4-acetylbenzoic acid in a similar manner as described in Ex-3B. Dark Yellow solid, 52%, mp 206-208-C. ¹H-NMR (300 MHz, DMSO-d₆, major isomer) δ 8.30 (s, 1H), 8.25 (d, 2H, J=7.8 Hz), 8.07-8.12 (m, 3H), 7.94 (d, 1H, J=15.6 Hz), 7.68 (d, 1H, J=3.3 Hz), 7.52 (d, 1H, J=5.1 Hz), 7.14 (dd, 1H, J=5.1, 3.3 Hz), 6.86 (s, 1H), 5.05 (m, 1H), 4.34 (m, 1H), 4.00 (s, 3H), 3.40-3.46 (m, 2H), 2.81 (s, 3H), 2.40-2.44 (m, 1H), 2.16-2.27 (m, 2H), 1.81-2.00 (m, 4H). MS (ESI) m/z=492 ([M+H]⁺, 100%). Anal. Calcd. for C₂₈H₃₀ClNO₅S—Y2H₂O: C, 60.59; H, 5.99; N, 2.52; S, 5.78. Found: C, 60.70; H, 5.85; N, 2.64; S, 6.15.

### Example 36

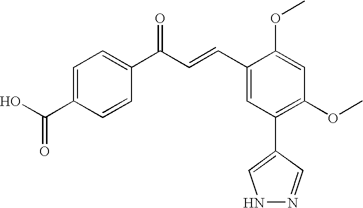

4-{3E-[2,4-Dimethoxy-5-(1H-pyrazol-4-yl)-phenyl]-acryloyl}-benzoic acid

Ex-36A: A solution of 4-(4,4,5,5-tetramethyl-[1,3,2]dioxaborolan-2-yl)-1H-pyrazole (0.33g, 1.70 mmol) and di-tert-butyl dicarbonate (0.51g, 2.34 mmol) in dichloromethane (10 mL) was allowed to stir overnight at ambient temperature. The solution was then washed with saturated solution of sodium bicarbonate and brine, dried over sodium sulfate, and concentrated. The crude product of 4-(4,4,5,5-tetramethyl-[1,3,2]dioxaborolan-2-yl)-pyrazole-1-carboxylic acid tert-butyl ester (0.61g) was used in next step without further purification.

Ex-36B: To a mixture of 2,4-dimethoxy-5-bromo-benzaldehye (0.28g, 1.13 mmol), 4-(4,4,5,5-tetramethyl-[1,3,2]dioxaborolan-2-yl)-pyrazole-1-carboxylic acid tert-butyl ester (Ex-36A, 0.61g, 1.70 mmol), bis(tri-tert-butylphosphine)palladium (43 mg, 0.085 mmol) and potassium fluoride (0.24g, 4.08 mmol) was added degassed tetrahydrofuran (15 mL). The reaction mixture was heated at 60° C. for one day. Additional potassium fluoride (0.24g, 4.08 mmol) and water (20 μL) were added. The reaction mixture continued to stir at 60° C. for another 8 hours. The reaction was then quenched by water. The aqueous solution was extracted with ethyl acetate. The solution of ethyl acetate was washed with saturated solution of sodium bicarbonate, brine, dried over sodium sulfate and concentrated. The crude product was purified by flash chromatography. Elution with ethyl acetate (50%, v/v, in hexane) afforded 4-(5-formyl-2,4-dimethoxy-phenyl)-pyrazole-1-carboxylic acid tert-butyl ester (0.15g, 40%) as white solid. ¹H NMR (CDCl₃) δ 10.35 (s, 1H), 8.43 (s, 1H), 8.09 (s, 1H), 8.02 (s, 1H), 6.52 (s, 1H), 4.02 (s, 3H), 3.99 (s, 3H), 1.68 (s, 9H). MS m/z=333 ([M+H]⁺, 100%).

Ex-36C: The title compound was prepared by condensing 2,4-dimethoxy-5-(1H-pyrazol-4-yl)-benzaldehyde (Ex-36B) and 4-acetylbenzoic acid in a similar manner as described in Ex-3B. Yellow solid, mp>250-C. ¹H-NMR (DMSO-d₆) δ 12.42 (bs, 1H), 8.20-8.03 (m, 8H), 7.85 (d, J=16.1 Hz), 6.74 (s, 1H), 3.95 (s, 3H), 3.94 (s, 3H). MS m/z=379 ([M+H]⁺, 100%).

### Example 37

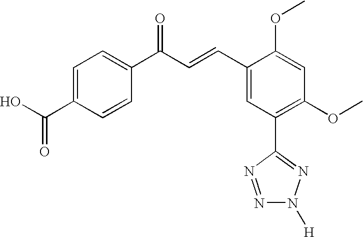

4-{3E-[2,4-Dimethoxy-5-(2H-tetrazol-5-yl)-phenyl]-acryloyl}-benzoic acid

Ex-37A: A solution of 2-(5-bromo-2,4-dimethoxy-phenyl)-[1,3]dioxolane (Ex-12A, 1.16 g, 4.9 mmol), sodium azide (641.3 mg, 9.86), and zinc bromide (552.2 mg, 2.46 mmol) in water (14 mL) and isopropanol (17 mL) were mixed and refluxed for 18 hours. The reaction mixture was quenched with 3N HCl (60 mL) and extracted with ethyl acetate (2×75 mL). The organic ws concentrated to a white solid. The solid was stirred in 0.25N NaOH (100 mL) for one hour. The suspension was filtered and the filtrate was collected and acidified with 1N HCl to a pH of 2. The aqueous solution was extracted with ethyl acetate:THF (40%). The organics were collected and concentrated to a crude brown solid of 2,4-dimethoxy-5-(2H-tetrazol-5-yl)-benzaldehyde (77.8 mg, 7%). ¹H-NMR (DMSO-d₆) δ 10.09 (s, 1H), 7.97 (s, 1H), 6.89 (s, 1H), 4.04 (s, 3H), 4.02 (s, 3H). MS m/z=234 ([M]+, 94%), 191 (100%).

Ex-37B: The title compound was prepared by condensing 2,4-dimethoxy-5-(2H-tetrazol-5-yl)-benzaldehyde (Ex-37A) and 4-acetylbenzoic acid in a similar manner as described in Ex-3B. Yellow solid, 19% yield, mp 218° C. (dec). ¹H-NMR (DMSO-d₆) δ 8.58 (s, 1H), 8.20 (d, 2H), 8.03 (m, 3H), 7.85 (d, 1H), 6.90 (s, 1H), 4.04 (s, 3H), 4.02 (s, 3H). MS m/z=422 ([M+CH₃CN+H]⁺, 100%). HRMS m/z: calc. 381.1199, found 381.1184.

### Example 38

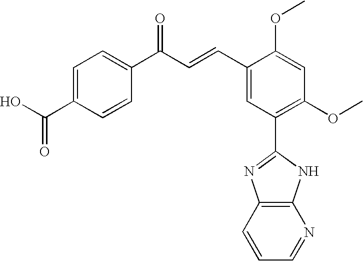

4-{3E-[5-(3H-Imidazo[4,5-b]pyridin-2-yl)-2,4-dimethoxy-phenyl]-acryloyl}-benzoic acid

Ex-38A: To a suspension of 2,4-dimethoxybenzoic acid (0.36 g, 2 mmol) and 8 ml of POCl₃ in a 50 ml of a round-bottom flask, 2,3-diaminopyridine (0.22 g, 2 mmol) was added. The mixture was heated to reflux for 4 hours and then cooled to room temperature. The reaction mixture was then concentrated to remove most of the POCl₃. The residue was carefully treated with 1N HCl at 0° C. using a water-ice bath, then neutralized with NaOH (50%). The off-white solid was filtered to give 2-(2,4-dimethoxy-phenyl)-3H-imidazo[4,5-b]pyridine (0.44 g, 88%). ¹H-NMR (DMSO-d₆) δ 8.28-8.36 (m, 2H), 7.97 (d, J=8 Hz, 1H), 7.21-7.25(m, 1H), 6.80 (s, 1H), 6.78 (d, J=9 Hz, 1H), 4.05(s, 3H), 3.91 (s, 3H). HRMS (ES+) Calcd. for C₂₄H₁₉N₃O₅: 430.1403. Found: 430.1414.

Ex-38B: To a suspension of 2-(2,4-dimethoxy-phenyl)-3H-imidazo[4,5-b]pyridine (0.44 g, 1.7 mmol) in 20 ml of CH₂Cl₂, 1, 1-dichlorodimethyl ether (0.55 g, 4.8 mmol) was added. The mixture was cooled to 0° C. with a water-ice bath, and 7 ml (7 mmol) of TiCl₄ (1.0 m in CH₂Cl₂) was added dropwise. The mixture was stirred at 0° C. for 2 hrs, then room temperature for overnight. The reaction mixture was poured into ice-water and the precipitate was filtered to give 0.31 g (63%) of 5-(3H-imidazo[4,5-b]pyridin-2-yl)-2,4-dimethoxy-benzaldehyde as a white solid. ¹H-NMR (DMSO-d₆) δ 10.22 (s, 1H), 8.67(s, 1H), 8.56 (d, J=5 Hz, 1H), 8.44 (d, J=8 Hz, 1H), 7.57-7.61(m, 1H), 6.97 (s, 1H), 4.19(s, 3H), 4.06 (s, 3H). HMRS (EI) calcd. for C₁₅H₁₃N₃O₃: 283.0957; found: 283.0952.

Ex:38C: The title compound was prepared by condensing 5-(3H-imidazo[4,5-b]pyridin-2-yl)-2,4-dimethoxy-benzaldehyde (Ex-38B) and 4-acetylbenzoic acid in a similar manner as described in Ex-3B. Yellow solid, mp 222-224° C., 60% yield. ¹H-NMR (DMSO-d₆) δ 8.75 (s, 1H), 8.38-8.40 (m, 1H), 8.18 (d, J=9 Hz, 2H), 7.99-8.08(m, 4H), 7.83(d, J=15 Hz, 1H), 7.28-7.33(m, 1H), 6.91 (s, 1H), 4.11 (s, 3H), 4.04 (s, 3H). MS m/z=430 ([M+H]⁺).

### Example 39

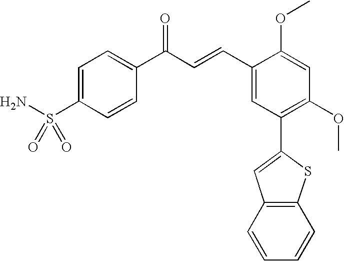

4-[3E-(5-Benzo[b]thiophen-2-yl-2,4-dimethoxy-phenyl)-acryloyl]-benzenesulfonamide

Ex-39: To a solution of 4-acetyl-benzsulfonamide (0.20g, 1.0 mmol) and 5-benzo[b]thiophene-2-yl-2,4-dimethoxyphenylbenzaldehyde (Ex-3A, 0.31 g, 1.05 mmol) in DMF (5 mL) and methanol (2 mL) was added lithium methoxide (0.15g, 4.0 mmol). The reaction mixture was allowed to stir at ambient temperature. The reaction was quenched with water (30 mL) after 2 hours. The aqueous solution was acidified to pH 4 with HCl (3 M) and extracted with ethyl acetate. The combined solution of ethyl acetate was subsequently washed with brine, dried (Na₂SO₄) and concentrated. The solid residue was stirred in ethanol (10 mL) for 1.5 hours, filtered, washed with aqueous ethanol (50%) and dried in vacuo. The title compound was obtained as a yellow solid (0.3g, 63%), mp 204-205° C. (dec.). ¹H-NMR (DMSO-d₆) δ 8.35 (s, 1H), 8.27 (d, J=7.7 Hz, 2H), 8.06 (d, J=16.0 Hz, 1H), 7.97-7.92 (m, 4H), 7.88 (d, J=6.6 Hz, 1H), 7.81 (d, J=7.4 Hz, 1H), 7.53 (s, 2H), 7.37-7.27 (m, 2H), 6.85 (s, 1H), 4.09 (s, 3H), 4.03 (s, 3H).

### Example 40

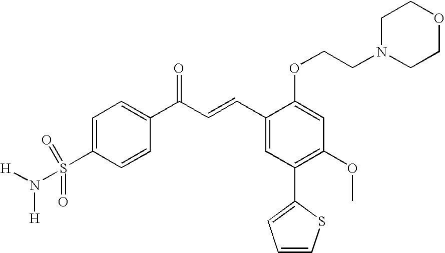

4-13E-[4-Methoxy-2-(2-morpholin-4-yl-ethoxy)-5-thiophen-2-yl-phenyl]-acryloyl}-benzenesulfonamide

Ex-40: 4-Acetyl-benzenesulfonamide (0.10 g, 0.29 mmol) and 4-acetylbenzenesulfonamide (0.057 g, 0.29 mmol) were dissolved in a dimethylformamide-methanol solution (2.0 mL, 7:3). After complete dissolution, lithium methoxide (0.044 g, 1.2 mmol) was added and the resulting orange slurry was stirred in the dark at room temperature for 4 h. Upon completion, as determined by HPLC, the mixture was diluted with water (15 mL) and extracted with ethyl acetate (3×25 mL). The combined organic extracts were dried over sodium sulfate and evaporated to dryness. The crude oil was taken up in ethanol (2 mL) and warmed to 60° C. to obtain complete dissolution and allowed to cool to room temperature. The resulting precipitate was collected on filter paper and dried in vacuo to yield 0.13 g (82%) of the title compound as a yellow solid, mp 186-188-C. ¹H-NMR (300 MHz, DMSO-d₆) δ 8.23-8.28 (m, 3H), 7.93-8.09 (m, 4H), 7.66 (d, 1H, J=3.0 Hz), 7.56 (brs, 1H), 7.52 (d, 1H, J=5.1 Hz), 7.13 (dd, 1H, J=5.1, 3.0 Hz), 6.89 (s, 1H), 4.34 (t, 2H, J=6 Hz), 4.01 (s, 3H), 3.54-3.58 (m, 4H), 2.83 (t, 2H, J=6 Hz), 2.51-2.53 (m, 4H). MS (ESI) m/z=529 ([M+H]⁺, 100%). Anal. Calcd. for C₂₆H₂₈N₂O₆S₂: C, 59.07; H, 5.34; N, 5.30; S, 12.13. Found: C, 58.90; H, 5.38; N, 5.37; S, 12.01.

### Example 41

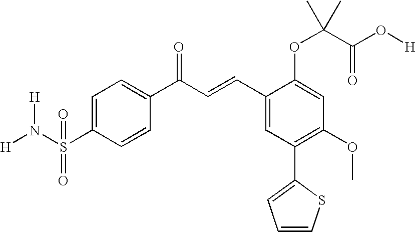

2-{5-Methoxy-2-[3-oxo-3-(4-aminosulfonyl-phenyl)-E-propenyl]-4-thiophen-2-yl-phenoxy}-2-methyl-propionic acid

Ex-41: The title compound was prepared by condensing 4-acetyl-benzenesulfonamide and 2-(2-formyl-5-methoxy-4-thiophen-2-yl-phenoxy)-2-methyl-propionic acid (Ex-21B) in a similar manner as described in Ex-3B. Yellow solid, mp 164-165° C., 85% yield. ¹H-NMR (300 MHz, DMSO-d₆) δ 8.21-8.28 (m, 3H), 7.96-8.12 (m, 4H), 7.67 (d, 1H, J=3.0 Hz), 7.56 (brs, 3.0H), 7.14 (dd, 1H, J=5.7, 3.0 Hz), 6.57 (s, 1H), 3.88 (s, 3H), 1.66 (s, 6H). MS (ESI) m/z=502 ([M+H]⁺, 100%). Anal. Calcd. for C₂₄H₂₃NO₇S₂: C, 57.47; H, 4.62; N, 2.79; S, 12.79. Found: C, 57.70; H, 4.74; N, 2.85; S, 12.51.

### Example 42

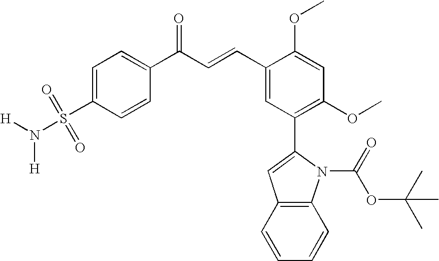

2-12,4-Dimethoxy-5-[3-oxo-3-(4-aminosulfonyl-phenyl)-E-propenyl]-phenyl)-indole-1-carboxylic acid tert-butyl ester

Ex-42: The title compound was prepared by condensing 4-acetyl-benzenesulfonamide and 2-(5-formyl-2,4-dimethoxy-phenyl)-indole-1-carboxylic acid tert-butyl ester in a similar manner as described in Ex-3B. Yellow solid, 40% yield, mp 120-122° C. ¹H-NMR (CDCl₃) δ 8.01-8.19 (m, 6H), 7.68 (s, 1H), 7.56 (d, J=8 Hz, 1H), 7.46(d, J=16 Hz, 1H), 7.21-7.35(m, 2H), 6.53 (d, J=14 Hz, 2H), 5.01(s, 2H), 4.00 (s, 3H), 3.85(s, 3H), 1.42 (s, 9H). MS m/z=563 ([M+H]⁺). HRMS (ES+) Calcd. for C₃₀H₃₀N₂O₇S: 563.1852. Found: 563.1862.

### Example 43

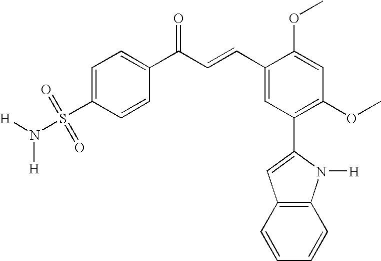

4-{3E-[5-(1N-Indol-2-yl)-2,4-dimethoxy-phenyl]-acryloyl}-benzenesulfonamide

Ex-43: The title compound was prepared by condensing 4-acetyl-benzenesulfonamide and 5-(1H-indol-2-yl)-2,4-dimethoxy-benzaldehyde (Ex-23A) in a similar manner as described in Ex-3B. Red solid, 70% yield, mp 185-187° C. ¹H-NMR (DMSO-d₆) δ 11.15 (br, s, 1H), 8.33(s, 1H), 8.24 (d, J=8 Hz, 2H), 8.07 (d, J=15 Hz, 1H), 7.98 (d, J=8 Hz, 2H), 7.80(d, J=15 Hz, 1H), 7.41-7.55(m, 4H), 7.03-7.08 (m, 1H), 6.93-6.99 (m, 2H), 6.83 (s, 1H), 4.04(s, 3H), 3.99(s, 3H). MS m/z=463 ([M+H]⁺). HRMS (ES+) Calcd. for C₂₅H₂₂N₂O₅S: 463.1327. Found: 463.1316.

### Example 44

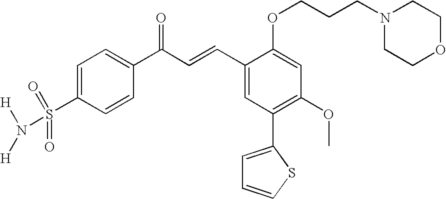

4-{3E-[4-Methoxy-2-(3-morpholin-4-yl-propoxy)-5-thiophen-2-yl-phenyl]-acryloyl}-benzenesulfonamide

Ex-44: The title compound was prepared by condensing 4-acetyl-benzenesulfonamide and 4-methoxy-2-(3-morpholin-4-yl-propoxy)-5-thiophen-2-yl-benzaldehyde (Ex-28A) in a similar manner as described in Ex-3B. Yellow solid, 48% yield, mp 193-196° C. ¹H-NMR (DMSO-d₆) δ 8.24 (m, 3H), 8.06 (s, 1H), 7.96 (d, 2H), 7.89 (d, 1H), 7.63 (d, 1H), 7.51 (m, 1H), 7.10 (dd, J=3, 4 Hz, 1H), 6.81 (s, 1H), 4.23 (t, 2H), 3.98(s, 3H), 3.55 (t, 4H), 2.47 (m, 2H), 2.35(t, 4H), 1.98(q, 2H). MS m/z=542 ([M]+, 38%), 100 (100%). Anal. calculated for C₂₇H₃₀N₂O₆S₂.⅗H₂O: C, 58.59, H, 5.68, S, 11.59; found C: 58.59, H: 5.55, S: 11.40.

### Example 45

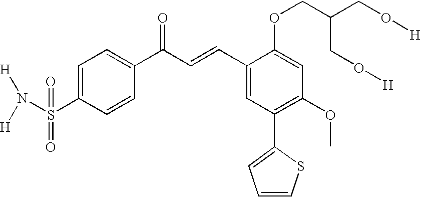

4-{3E-[2-(3-Hydroxy-2-hydroxymethyl-propoxy)-4-methoxy-5-thiophen-2-yl-phenyl]-acryloyl}-benzenesulfonamide

Ex-45: 2-(3-Hydroxy-2-hydroxymethyl-propoxy)-4-methoxy-5-thiophen-2-yl-benzaldehyde (Ex-26B) (8.0 g, 24.8 mmol) and 4-acetylbenzenesulfonamide (4.9 g, 24.8 mmol) were dissolved in a dimethylformamide-methanol solution (170 mL, 7:3). After complete dissolution, lithium methoxide (3.8 g, 99.2 mmol) was added and the resulting red-orange slurry was stirred in the dark at room temperature for 3 h. Upon completion, as determined by HPLC, the mixture was diluted with water (500 mL) and extracted with ethyl acetate (6×200 mL). The combined organic extracts were dried over sodium sulfate and evaporated to dryness. The crude oil was taken up in ethanol (150 mL) and warmed to 60° C. to obtain complete dissolution and allowed to cool to room temperature. The resulting precipitate was collected on filter paper and dried in vacuo to yield 7.0 g (60%) of the title compound as a light orange solid, mp 123-124° C. ¹H-NMR (300 MHz, DMSO-d₆) δ 8.25-8.29 (m, 3H), 7.90-8.11 (m, 4H), 7.66 (d, 1H. J=3.0 Hz), 7.56 (brs, 1H), 7.52 (d, 1H. J=5.1 Hz), 7.13 (dd, 1H. J=5.1, 3.0 Hz), 6.88 (s, 1H), 4.67 (t, 2H, J=10.8 Hz), 4.24 (d, 2H, J=6.0 Hz), 4.00 (s, 3H), 3.54-3.65 (m, 4H), 2.09-2.13 (m, 1H). MS (ESI) m/z=504 ([M+H]⁺, 100%). Anal. Calcd. C₂₄H₂₅NO₇S₂H₂O: C, 57.24; H, 5.00; N, 2.78; S, 12.73. Found: C, 56.72; H, 5.27; N, 2.71; S. 12.11.

### Example 46

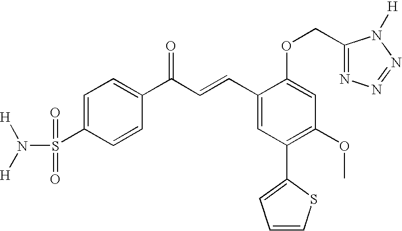

4-{3E-[4-Methoxy-2-(1H-tetrazol-5-ylmethoxy)-5-thiophen-2-yl-phenyl]-acryloyl}-benzenesulfonamide

Ex-46A: (2-Acetyl-5-methoxy-4-thiophen-2-yl-phenoxy)-acetonitrile was prepared in an analogous fashion as described in Ex-1C using iodoacetonitrile. The crude solid was slurried in ethyl acetate (50 mL) to remove residual impurities. The resulting solid was collected on filter paper and dried in vacuo to give the expected product as an orange solid (70%), mp 175-176° C. ¹H-NMR (300 MHz, CDCl₃) δ 10.29 (s, 1H), 8.17 (s, 1H), 7.48 (d, 1H, J=3.6 Hz), 7.35 (d, 1H, J=5.1 Hz), 7.10 (dd, 1H, J=5.1, 3.6 Hz), 6.64 (s, 1H), 4.96 (s, 2H), 4.06 (s, 3H). MS (EI) m/z=273 ([M]⁺, 99%), 233 (100%). Anal. Calcd. for C₁₄H₁₁NO₃S: C, 61.52; H, 4.06; N, 5.12; S, 11.73. Found: C, 61.65; H, 4.20; N, 5.16; S, 11.59.

Ex-46B: (2-Acetyl-5-methoxy-4-thiophen-2-yl-phenoxy)-acetonitrile (Ex-46A, 0.30 g, 1.1 mmol) was slurried in a mixture of water:isopropanol (3 mL, 2:1) to obtain a well-dispersed solution. Sodium azide (0.079 g, 1.2 mmol) followed by zinc bromide (0.25 g, 1.1 mmol) were added and the reaction was heated to reflux and vigorously stirred for 24 h. Additional solvent (1 mL, 1:1 water:isopropanol) was added after 10 h at reflux due to evaporation. The reaction was diluted with an ethyl acetate:tetrahydrofuran mixture (25 mL, 2:1) and a 3 N HCl solution (10 mL) and vigorously stirred until a homogenous solution was obtained (1 h). The layers were separated and the aqueous was extracted with ethyl acetate (3×50 mL). The combined organic extracts were dried over sodium sulfate and concentrated to a dark green solid. Silica gel chromatography (15% methanol/methylene chloride containing 1% acetic acid) gave 0.22 g (65%) of 4-methoxy-2-(1H-tetrazol-5-ylmethoxy)-5-thiophen-2-yl-benzaldehyde as a pale green solid. ¹H-NMR (300 MHz, DMSO-d₆) δ 10.33 (s, 1H), 7.97 (s, 1H), 7.52-7.56 (m, 2H), 7.10-7.12 (m, 2H), 5.81 (s, 2H), 4.05 (s, 3H). MS (ESI) m/z=317 ([M+H]⁺, 100%). HRMS (ESI) Calcd. for C₂₇H₂₅NO₇S: 317.0708. Found: 317.0712.

Ex-46: The title compound was prepared by condensing 4-acetylbenzenesulfonamide (Ex-26A) and 4-methoxy-2-(1H-tetrazol-5-ylmethoxy)-5-thiophen-2-yl-benzaldehyde (Ex-46A) in a similar manner as described in Ex-3B. Yellow solid, mp 163-164° C. (dec), 60% yield. ¹H-NMR (300 MHz, DMSO-d₆) δ 8.31-8.34 (m, 3H), 7.92-8.15 (m, 4H), 7.70 (d, 1H, J=4.0 Hz), 7.54 (m, 3H), 7.15-7.17 (m, 1H), 6.92 (s, I), 4.64 (brs, 2H), 4.03 (s, 5H). MS (ESI) m/z=498 ([M+H]⁺, 100%). Anal. Calcd. for C₂₂H₁₉N₅O₅S₂.1½H₂O: C, 50.37; H, 4.23; N, 13.35; S, 12.23. Found: C, 50.48; H, 4.24; N, 12.95; S, 12.35.

### Example 47

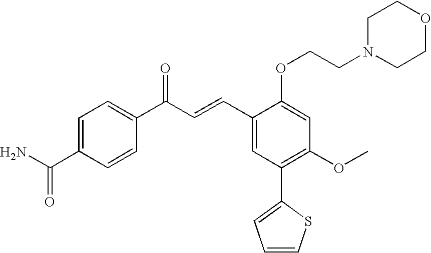

4-{3E-[4-Methoxy-2-(2-morpholin-4-yl-ethoxy)-5-thiophen-2-yl-phenyl]-acryloyl}-benzamide

Ex-47A: To a solution of 4-acetyl-benzoic acid (0.5g, 3.05 mmol) in tetrahydrofuran (10 mL) was added carbonyldiimidazole (0.74g, 4.75 mmol). The solution was allowed to stir at ambient temperature for one hour and cooled to 0° C. followed by addition of ammonia (28% in water, 3 mL, 21 mmol). The solution was continued to stir at 0° C. for another one hour. The solvent was removed under reduced pressure. The residue was treated with water, filtered, washed with water, dried in vacuo to give 4-acetyl-benzamide (0.25g, 50%) as a white solid. ¹H NMR (DMSO-d₆) δ 8.11 (bs, 1H), 8.00 (d, J=9 Hz, 2H), 7.95 (d, J=9 Hz, 2H), 7.53 (bs, 1H), 2.59 (s, 3H).

Ex-47B: To a solution of 4-acetyl-benzamide (Ex-47A, 0.25g, 1.53 mmol) and 2-(2-morpholin-4-yl-ethoxy)-4-methoxy-5-thiophen-2-yl-benzaldehyde (Ex-22A, 0.53g, 1.53 mmol) in DMF (7 mL) and methanol (3 mL) was added lithium methoxide. The solution was allowed to stir at ambient temperature. The reaction was quenched with water after 2 hours. The aqueous solution was extracted with ethyl acetate. The combined extract was washed with NaHCO₃, NH₄Cl, brine, dried (Na₂SO₄) and concentrated. The residue was stirred in ethanol overnight to afford the title compound as a yellow solid (0.43g, 57%), mp 183-184° C. ¹H-NMR (CDCl₃) δ 8.09-8.04 (m, 3H), 7.93 (d, J=8.3 Hz, 2H), 7.87 (s, 1H), 7.57 (d, J=15.7 Hz, 1H), 7.42 (d, J=3.9 Hz, 1H), 7.32 (d, 4.4 Hz, 1H), 7.11-7.08 (m, 1H), 6.55 (s, 1H), 6.25 (bs, 1H), 5.75 (bs, 1H), 4.25 (t, J=5.9 Hz, 2H), 3.98 (s, 31H), 3.71 (t, J=4.2 Hz, 4H), 2.92 (t, J=5.7 Hz, 2H), 2.59 (t, J=4.6 Hz, 4H). MS m/z=493 ([M+H]⁺, 100%).

### Example 48

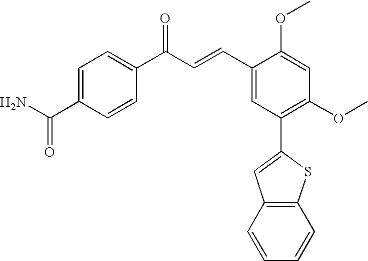

4-[3E-(5-Benzo[b]thiophen-2-yl-2,4-dimethoxy-phenyl)-acryloyl]-benzamide

Ex-48: To a solution of 4-acetyl-benzamide (0.3g, 1.84 mmol) and 5-(benzo[b]thein-2yl)-2,4-dimethoxybenzaldehyde (0.55 g, 1.84 mmol) in a mixture of N,N-dimethylformamide (7 mL) and methanol (3 mL) was added lithium methoxide (0.14 g, 3.68 mmol). The reaction mixture was allowed to stir at ambient temperature for 9 hours. The resulting precipitate was collected by filtration, washed with methanol, dried in vacuo to obtain the title compound as a yellow solid (5.56g, 68%), mp 240-241° C. ¹H-NMR (DMSO-d₆) δ 8.37 (s, 1H), 8.19 (d, J=7.8 Hz, 2H), 8.12 (d, J=15.3 Hz, 1H), 8.04-7.91 (m, 6H), 7.83 (d, J=7.5 Hz, 1H), 7.55 (s, 1H), 7.36-7.30 (m, 2H), 6.87 (s, 1H), 4.04 (s, 3H), 4.01 (s, 3H). MS m/z=444 ([M+H]⁺, 100%).

### Example 49

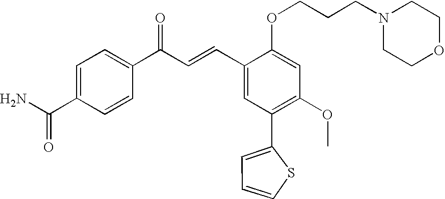

4-{3E-[4-Methoxy-2-(3-morpholin-4-yl-propoxy)-5-thiophen-2-yl-phenyl]-acryloyl}-benzamide

Ex-49: The title compound was prepared by condensing 4-Acetyl-benzamide (Ex-47A) and 4-methoxy-2-(3-morpholin-4-yl-propoxy)-5-thiophen-2-yl-benzaldehyde (Ex-28A) in a similar manner as described in Ex-47B. Orange solid, mp 81-83-C. ¹H-NMR (CDCl₃) δ 8.08 (m, 3H), 7.94 (d, 2H), 7.86 (s, 1H), 7.56 (d, 1H), 7.41 (d, 1H), 7.32 (d, 1H), 7.10 (m, 1H), 6.55 (s, 1H), 4.19 (t, 2H), 3.99(s, 3H), 3.72 (t, 4H), 2.59 (t, 2H), 2.12 (t, 4H), 1.98(quintet, 2H). MS tn/z=506 ([M]+, 34%), 100(100%). 28%. Anal. calculated for C₂₈H₃₀N₂O₅S.⅖H₂O: C, 65.45, H, 6.04, S, 6.24; found C: 65.30, H: 6.16, S: 6.17.

### Example 50

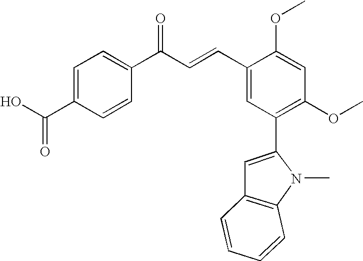

4-{3E-[2,4-Dimethoxy-5-(d-methyl-1H-indol-2-yl)-phenyl]-acryloyl}-benzoic acid

Ex-50A: To a solution of N-methyl indole (1.3 g, 10 mmol) in 50 ml THF, t-BuLi (1.7m in THF, 7.1 ml, 12 mmol) was slowly added at 0° C. under nitrogen. The mixture was stirred at room temperature for 1 hr, BEt₃ (1.0 M in THF, 12 ml, 12 mmol) was added, and the mixture stirred for another 1 hr at room temperature. Then, PdCl₂(PPh₃)₂ (0.35 g, 0.5 mmol) and 5-bromo-2,4-dimethoxybenzaldehyde (3.7g, 15 mmol) were added, and the mixture was heated to about 60° C. for 30 minutes. The reaction mixture was poured into 50 ml 10% NaOH and treated with 30% H₂O₂ and then stirred for 10 minutes. The mixture was extracted with EtOAc and combined organic phase was washed with H₂O and brine, dried over MgSO₄, and absorbed to small amount of silica gel. Column chromatography (EtOAc: Hexane, 1:2) gave 0.72 g (25%) 2,4-dimethoxy-5-(1-methyl-1H-indol-2-yl)-benzaldehyde. ¹H-NMR (CDCl₃) δ 10.33 (s, 1H), 7.84 (s, 1H), 7.60 (d, J=8 Hz, 1H), 7.31 (d, J=8 Hz, 1H), 7.18-7.24 (m, 1H), 7.07-7.12(m, 1H), 6.53 (s, 1H), 6.46(s, 1H), 4.00 (s, 3H), 3.89 (s, 3H), 3.53 (s, 3H). HRMS (EI) Calcd. for C₁₈H₁₇NO₃: 295.1208. Found: 295.1202.

Ex-50B: The title compound was prepared by condensing 4-acetylbenzoic acid and 2,4-dimethoxy-5-(1-methyl-1H-indol-2-yl)-benzaldehyde (Ex-50A) in a similar manner as described in Ex-3B. Yellow solid, 87% yield, mp 157-160° C. ¹H-NMR (DMSO-d₆) δ 8.17 (d, J=8 Hz, 2H), 8.08 (d, J=15 Hz, 1H), 7.99-9.02 (m 3H), 7.83 (d, J=15 Hz, 1H), 7.52 (d, J=8 Hz, 1H), 7.42 (d, J=8 Hz, 1H), 7.10-7.15 (m, 1H), 6.99-7.04(m, 1H), 6.85 (s, 1H), 6.42(s, 1H), 4.01 (s, 3H), 3.88 (s, 3H), 3.50 (s, 3H). MS m/z=442 ([M+H]⁺, 100%). HRMS (ES+) Calcd. for C₂₇H₂₃NO₅: 442.1654. Found: 442.1633.

### Example 51

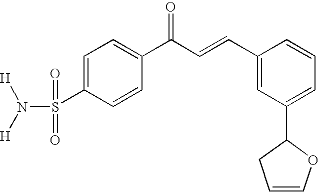

4-{3-[3E-(2,3-Dihydro-furan-2-yl)-phenyl]-acryloyl}-benzenesulfonamide

Ex-51A: 5-Bromobenzaldehyde (0.5 g, 2.7 mmol) and 2,3-dihydrofuran (0.56 g, 8.1 mmol) were dissolved in dioxane (5.0 mL). Nitrogen was bubbled into the solution for 15 min followed by the sequential addition of cesium carbonate (0.96 g, 2.9 mmol) and bis(tri-t-butylphosphine)palladium(0) (0.014 g, 0.027 mmol). The solution was immediately heated to 45° C. and aged for 24 h. Upon completion, as determined by HPLC, the reaction was diluted with water (20 mL) and extracted with ethyl acetate (3×20 mL). The combined organic extracts were dried over sodium sulfate and concentrated to a brown oil. Silica gel chromatography (ethyl acetate/hexanes, 1:9) gave 0.18 g (40%) of 3-(2,3-dihydro-furan-2-yl)-benzaldehyde as a clear, colorless oil. ¹H-NMR (300 MHz, CDCl₃) δ 10.03 (s, 1H), 7.88 (s, 1H), 7.82 (d, 1H, J=7.2 Hz), 7.62-7.64 (m, 1H), 7.53 (t, 1H, J=7.2 Hz), 6.48 (q, 1H, J=Hz), 5.60 (dd, 1H, J=8.1, 10.8 Hz), 4.98 (q, 1H, J=3.3 Hz), 3.15 (ddt, 1H, J=15.0, 8.1, 2.5 Hz), 2.59 (ddt, 1H, J=15.0, 8.1, 2.5 Hz). MS (EI) m/z=174 ([M]+, 100%). HRMS (EI) Calcd. for C₁₁H₁₀O₂: 174.0681. Found: 174.0677.

Ex-51B: The title compound was prepared by condensing 4-acetylbenzenesulfonamide (Ex-26A) and 3-(2,3-dihydro-furan-2-yl)-benzaldehyde (Ex-51A) in a similar manner as described in Ex-3B. Tan solid, 40% yield, mp 152-153-C. ¹H-NMR (300 MHz, DMSO-d₆) δ 8.31 (d, 2H, J=7.5 Hz), 7.99 (d, 2H, J=7.5 Hz), 7.95 (d, 1H, J=15.8 Hz), 7.85 (brs, 3H), 7.78 (d, 1H, J=15.8 Hz), 7.57 (brs, 1H), 7.44-7.52 (m, 2H), 6.62 (q, 1H, J=2.4 Hz), 5.58 (dd, 1H, J=8.7, 10.8 Hz), 5.59 (q, 1H, J=2.4 Hz), 3.10 (ddt, 1H, J=15.0, 8.1, 2.5 Hz), 2.54 (ddt, 1H, J=15.0, 8.1, 2.5 Hz). MS (ESI) m/z=356 ([M+H]⁺, 100%). Anal. Calcd. for C₁₉H₁₇NO₄S.⅕H₂O: C, 63.56; H, 4.89; N, 3.90; S, 8.93. Found: C, 63.64; H, 4.88; N, 4.00; S, 8.71.

### Example 52

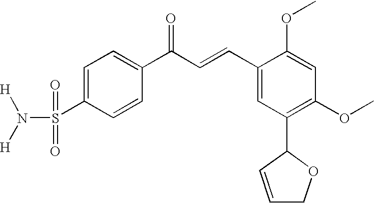

4-{3E-[5-(2,5-Dihydro-furan-2-yl)-2,4-dimethoxy-phenyl]-acryloyl}-benzenesulfonamide

Ex-52A: 5-Bromo-2,4-dimethoxybenzaldehyde (1.0 g, 4.0 mmol) and 2,3-dihydrofuran (0.85 g, 12.2 mmol) were dissolved in dioxane (10.0 mL). Nitrogen was bubbled into the solution for 15 min followed by the sequential addition of cesium carbonate (1.4 g, 4.5 mmol) and bis(tri-t-butylphosphine)palladium (0) (0.021 g, 0.041 mmol). The solution was immediately heated to 45° C. and aged for 72 h. Additional equivalents of cesium carbonate (0.70 g, 2.1 mmol), 2,3-dihydrofuran (0.85 g, 12.2 mmol), and Pd catalyst (0.0021 g, 0.0041 mmol) were added after 24 h and 48 h to drive the reaction to completion. Upon completion, as determined by HPLC, the reaction was diluted with water (30 mL) and extracted with ethyl acetate (3×30 mL). The combined organic extracts were dried over sodium sulfate and concentrated to an orange oil. Silica gel chromatography (ethyl acetate/hexanes, 1:2) afforded 0.32 g (50%) of 5-(2,5-dihydro-furan-2-yl)-2,4-dimethoxy-benzaldehyde as a pale yellow solid, mp 84-85° C. ¹H-NMR (300 MHz, CDCl₃) δ 10.29 (s, 1H), 7.79 (s, 1H), 6.42 (s, 1H), 5.99-6.06 (m, 2H), 5.89-5.92 (m, 1H), 4.80-4.87 (m, 1H), 4.71-4.77 (m, 1H), 3.95 (s, 3H), 3.92 (s, 3H). MS (EI) m/z=234 ([M]+, 100%). Anal. Calcd. C₁₃H₁₄O₄: C, 66.66; H, 6.02. Found: C, 66.49; H, 6.08.

Ex-52B: 5-(2,5-Dihydro-furan-2-yl)-2,4-dimethoxy-benzaldehyde (Ex-52A, 0.10 g, 0.43 mmol) and 4-acetylbenzenesulfonamide (Ex-26A, 0.085 g, 0.43 mmol) were dissolved in a dimethylformamide-methanol solution (2.9 mL, 7:3). After complete dissolution, lithium methoxide (0.065 g, 1.7 mmol) was added and the resulting orange slurry was stirred in the dark at room temperature for 4 h. Upon completion, as determined by HPLC, the mixture was diluted with water (15 mL) and extracted with ethyl acetate (3×20 mL). The combined organic extracts were dried over sodium sulfate and evaporated to dryness. The crude oil was taken up in ethanol (2 mL) and warmed to 60° C. to obtain complete dissolution and allowed to cool to room temperature. The resulting precipitate was collected on filter paper and dried in vacuo to yield 0.13 g (70%) of the title compound as a yellow solid, mp 194-195° C. 1H-NMR (300 MHz, DMSO-d₆) δ 8.23 (d, 2H, J=8.2 Hz), 8.03 (d, 1H, J=15.3 Hz), 7.97 (d, 2H, J=8.2 Hz), 7.69 (s, 1H), 7.65 (d, 1H, J=15.3 Hz), 7.55 (brs, 2H), 6.73 (s, 1H), 6.06-6.09 (m, 1H), 5.90-5.98 (m, 2H), 4.86-4.92 (m, 1H), 4.63-4.68 (m, 1H), 3.96 (s, 3H), 3.92 (s, 3H). MS (ESI) m/z=416 ([M+H]⁺, 100%). Anal. Calcd. C₂₁H₂₁NO₆S: C, 60.71; H, 5.09; N, 3.37; S, 7.72. Found: C, 60.95; H, 5.24; N, 3.46; S, 7.72.

### Example 53

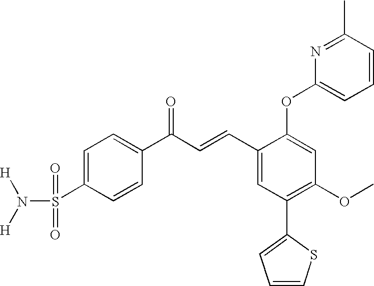

4-{3E-[4-Methoxy-2-(6-methyl-pyridin-2-yloxy)-5-thiophen-2-yl-phenyl]-acryloyl}-benzenesulfonamide

Ex-53A: To a solution of 2-hydroxy-4-methoxy-5-thiophen-2-yl-benzaldehyde (0.68 g, 2.9 mmol) and 2-bromo-6-methylpyridine (0.25 g, 1.4 mmol) in toluene (1.0 mL) was added 1-naphthoic acid (0.50 g, 2.9 mmol), 5A molecular sieves (0.36 g), cesium carbonate (0.94 g, 2.9 mmol), and copper (1) triflate-benzene complex (0.020 g, 0.036 mmol). The phenoxide crashed out of solution upon addition of cesium carbonate and additional toluene (1 mL) was added to facilitate stirring. The heterogeneous solution was immediately heated to 110° C. and aged for 24 h. Upon completion, as determined by HPLC, the reaction was diluted with a 5% sodium hydroxide solution (10 mL) and ethyl acetate (10 mL) and stirred for 30 min. The layers were separated and the aqueous layer was extracted with ethyl acetate (5×20 mL). The combined organic extracts were washed with a 50% brine solution (1×25 mL), brine (1×25 mL), dried over sodium sulfate and concentrated to an dark brown semi-solid. Silica gel chromatography (ethyl acetate/hexanes, 1:4) afforded 0.30 g (65%) of 4-methoxy-2-(6-methyl-pyridin-2-yloxy)-5-thiophen-2-yl-benzaldehydeas a light orange solid, mp 140-141° C. ¹H-NMR (300 MHz, CDCl₃) δ 10.21 (s, 1H), 8.23 (s, 1H), 7.64 (dd, 1H, J=7.8, 7.2 Hz), 7.52 (d, 1H, J=3.3 Hz), 7.35 (d, 1H, J=5.1 Hz), 7.10 (dd, 1H, J=5.1, 3.3 Hz), 6.94 (d, 1H, J=7.2 Hz), 6.78 (d, 1H, J=7.8 Hz), 6.75 (s, 1H), 3.92 (s, 3H), 2.44 (s, 3H). HRMS (EI) Calcd. for C₁₈H₁₅NO₃S: 325.0773. Found: 325.0775. Anal. Calcd. C₁₈H₁₅NO₃S: C, 66.44; H, 4.65; N, 4.30; S, 9.85. Found: C, 60.00; H, 4.58; N, 4.05; S, 9.84.

Ex-53B: 4-Methoxy-2-(6-methyl-pyridin-2-yloxy)-5-thiophen-2-yl-benzaldehyde (Ex-53A, 0.20 g, 0.62 mmol) and 4-acetylbenzenesulfonamide (Ex-26A, 0.12 g, 0.62 mmol) were dissolved in a dimethylformamide-methanol solution (4.2 mL, 7:3). After complete dissolution, lithium methoxide (0.093 g, 2.5 mmol) was added and the resulting orange slurry was stirred in the dark at room temperature for 3 h. Upon completion, as determined by HPLC, the mixture was diluted with water (10 mL) and extracted with ethyl acetate (3×20 mL). The combined organic extracts were dried over sodium sulfate and evaporated to dryness. The crude oil was taken up in ethanol (2 mL) and warmed to 60° C. to obtain complete dissolution and allowed to cool to room temperature. The resulting precipitate was collected on filter paper and dried in vacuo to yield 0.25 g (82%) of the title compound as a yellow solid, mp 164-165° C. ¹H-NMR (300 MHz, DMSO-d₆) δ 8.47 (s, 1H), 8.24 (d, 2H, J=8.1 Hz), 7.98 (d, 1H, J=15.3 Hz), 7.96 (d, 2H, J=8.1 Hz), 7.78-7.85 (m, 2H), 7.77 (d, 1H, J=15.3 Hz), 7.62 (d, 1H, J=5.1 Hz), 7.57 (s, 2H), 7.19 (dd, 1H, J=5.1, 3.6 Hz), 7.04 (d, 1H, J=7.5 Hz), 6.99 (s, 1H), 6.91 (d, 1H, J=8.4 Hz), 3.90 (s, 3H), 2.33 (s, 3H). Anal. Calcd. C₂₆H₂₂N₂O₅S₂: C, 61.64; H, 4.38; N, 5.53; S, 12.66. Found: C, 61.88; H, 4.47; N, 5.59; S, 12.62.

### Example 54

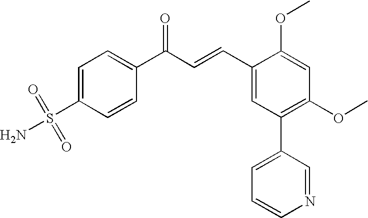

4-[3E-(2,4-Dimethoxy-5-pyridin-3-yl-phenyl)-acryloyl]-benzenesulfonamide

Ex-54A: 2,4-Dimethoxy-5-pyridin-3-yl-benzaldehyde was prepared in a similar manner as described in Ex-3A from pyridine-3-boronic acid and 5-bromo-2,4-dimethoxybenzaldehyde, 68% yield. ¹H-NMR (CDCl₃) δ 10.33 (s, 1H), 8.71 (d, J=1 Hz, 1H), 8.51-8.53(m, 1H), 7.81 (s, 1H), 7.74-7.78 (m, 1H), 7.27-7.31 (m, 1H), 6.52 (s, 1H), 3.99 (s, 3H), 3.91 (s, 3H). HMRS (EI) calcd. for C₁₄H₁₃NO₃: 243.0895; found: 243.0888.

Ex-54B: The title compound was prepared by condensing 2,4-dimethoxy-5-pyridin-3-yl-benzaldehyde (Ex-54A) and 4-acetyl-benzenesulfonamide (Ex-26A) in a similar manner as described in Ex-3B. Yellow solid, 51% yield, mp 253-255° C. ¹H-NMR (DMSO-d₆) δ 8.69 (d, J=1 Hz, 1H), 8.50 (d, J=4 Hz, 1H), 8.25 (d, J=9 Hz, 2H), 8.08 (d, J=15 Hz, 1H), 8.02 (s, 1H), 7.84-7.94(m, 4H), 7.51 (s, 2H), 7.40-7.44 (m, 1H), 6.82(s, 1H), 3.98 (s, 3H), 3.88 (s, 3H). MS m/z=424([M]+, 45%), 393 (100%). HMRS (EI) calcd. for C₂₂H₂₀N₂O₅S: 424.1093; found: 424.1100.

### Example 55

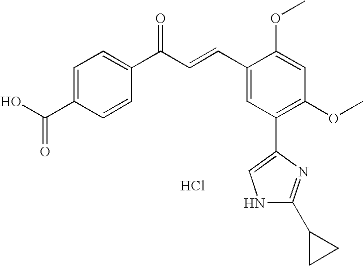

4-{3E-[5-(2-Cyclopropyl-1H-imidazol-4-yl)-2,4-dimethoxy-phenyl]-acryloyl}-benzoic acid, hydrochloride

Ex-55A: A solution of 2-bromo-1-(3,4-dimethoxy-phenyl)-ethanone (0.3g, 1.16 mmol), cyclopropanecarboxamidine (0.14g, 1.16 mmol) and sodium hydroxide (0.18g, 4.5 mmol) in ethanol was refluxed overnight. The solvent was removed under reduced pressure, the residue taken up to water. The aqueous solution was then extracted with dichloromethane which was subsequently washed with brine, dried over sodium bicarbonate and concentrated. The crude product was purified by flash chromatography. Elution with ethyl acetate (50%, v/v, in hexane) then methanol (10%, v/v in dichloromethane) afforded 2-cyclopropyl-4-(2,4-dimethoxy-phenyl)-1H-imidazole as white solid (0.15g, 53%): ¹HNMR (CDCl₃) δ 9.50 (bs, 1H), 7.63 (s, 1H), 7.20 (s, 1H), 6.57-6.53 (m, 2H), 3.93 (s, 3H), 3.03 (s, 3H), 1.97-1.93 (m, 1H), 1.00-0.94 (m, 4H). MS m/z=245 ([M+H]⁺, 100%).

Ex-55B: To a solution of 2-cyclopropyl-4-(2,4-dimethoxy-phenyl)-1H-imidazole (0.51 g, 2.09 mmol) was added dichloromethyl methyl ether (0.28 mL, 3.13 mmol) followed by addition of titanium tetrachloride (11.0M in dichloromethane, 8.4 mL, 8.4 mmol) dropwise at 0° C. The solution was allowed to warm up to ambient temperature and stir for 4.5 hours. The reaction mixture was then poured into ice. The aqueous layer was adjusted to pH 12 and extracted with dichloromethane. The combined solution of dichloromethane was washed with saturated solution of sodium bicarbonate, brine, dried over sodium sulfate and concentrated to afford 5-(2-cyclopropyl-1H-imidazol-4-yl)-2,4-dimethoxy-benzaldehyde which was used without further purification. ¹H NMR (DMSO-d₆) δ 13.95 (bs, 1H), 10.22 (s, 1H), 8.09 (s, 1H), 7.70 (s, 1H), 6.88 (s, 1H), 4.04 (s, 3H), 4.00 (s, 3H), 2.25 (m, 1H), 1.20 (m, 4H). MS m/z=245 ([M+H]⁺, 100%).

Ex-55C: The title compound was prepared by condensing 5-(2-cyclopropyl-1H-imidazol-4-yl)-2,4-dimethoxy-benzaldehyde (Ex-55B) and 4-acetylbenzoic acid in a similar manner as described in Ex-3B. Yellow solid, m.p. >240-C. ¹H NMR (DMSO-d₆) δ 13.31 (bs, 1H), 8.29 (d, J=8.9 Hz, 2H), 8.06-8.01 (m, 3H), 7.91 (s, 1H), 7.67 (s, 1H), 6.83 (s, 1H), 4.02 (s, 3H), 3.98 (s, 3H), 1.29-1.22 (m, 4H). MS m/z=419 ([M+H]⁺, 100%).

### Example 56

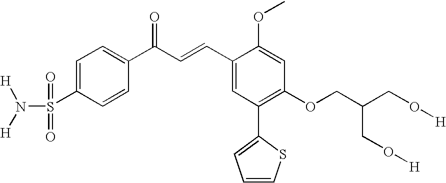

4-{3E-[4-(3-Hydroxy-2-hydroxymethyl-propoxy)-2-methoxy-5-thiophen-2-yl-phenyl]-acryloyl}-benzenesulfonamide

Ex-56: The title compound was prepared by condensing 4-(3-hydroxy-2-hydroxymethyl-propoxy)-2-methoxy-5-thiophen-2-yl-benzaldehyde (Ex-14C) and 4-acetyl-benzenesulfonamide in a similar manner as described in Ex-3B. Yellow solid, 72% yield, mp 191-192-C. ¹H-NMR (300 MHz, DMSO-d₆) δ 8.29-8.32 (m, 3H), 8.09 (d, 1H, J=16.0 Hz), 7.99 (d, 2H, J=8.1 Hz), 7.92 (d, 1H, J=16.0 Hz), 7.70 (d, 1H, J=3.3 Hz), 7.53-7.56 (m, 3H), 7.14 (dd, 1H, J=5.4, 3.3 Hz), 6.87 (s, 1H), 4.61 (t, 2H, J=5.1 Hz), 4.28 (d, 2H, J=5.1 Hz), 4.00 (s, 3H), 3.60-3.67 (m, 4H), 2.11-2.15 (m, 1H). MS (ESI) m/z=504 ([M+H]⁺, 100%). Anal. Calcd. for C₂₄H₂₅NO₇S₂.½H₂O: C, 56.23; H, 5.11; N, 2.73; S, 12.51. Found: C, 56.32; H, 5.06; N, 2.83; S, 12.55.

### Example 57

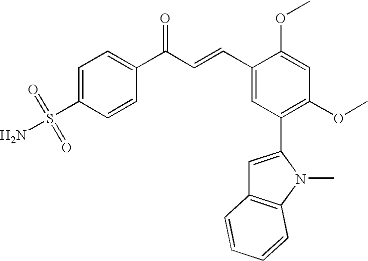

4-{3E-[2,4-Dimethoxy-5-(1-methyl-1H-indol-2-yl)-phenyl]-acryloyl}-benzenesulfonamide

Ex-57: The title compound was prepared by condensing 4-acetylbenzenesulfonamide (Ex-26A) and 2,4-dimethoxy-5-(1-methyl-1H-indol-2-yl)-benzaldehyde (Ex-50A) in a similar manner as described in Ex-3B. Yellow solid, 90% yield, mp 148-150° C. ¹H-NMR (CDCl₃) δ 8.17 (d, J=16 Hz, 1H), 8.09 (d, J=9 Hz, 2H), 8.01 (d, J=9 Hz, 2H), 7.68 (s, 1H), 7.64 (d, J=8 Hz, 1H), 7.47 (d, J=16 Hz, 1H), 7.35 (d, J=8 Hz, 1H), 7.22-7.26 (m, 1H), 7.11-7.16(m, 1H), 6.58 (s, 1H), 6.50(s, 1H), 4.92 (br, 2H), 4.02 (s, 3H), 3.90 (s, 3H), 3.58 (s, 3H). MS m/z=477 ([M+H]⁺, 100%). HRMS (ES+) Calcd. for C₂₆H₂₄NO₅S: 477.1484. Found: 477.1487.

### Example 58

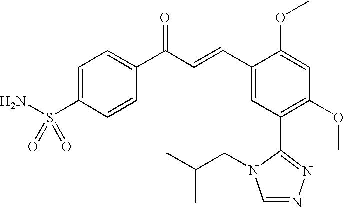

4-{3E-[5-(4-Isobutyl-4H-[1,2,4]triazol-3-yl)-2,4-dimethoxy-phenyl]-acryloyl}-benzenesulfonamide

Ex-58A: A solution of 2,4-dimethoxy-benzoic acid methyl ester (4.24g, 21.6 mmol) and hydrazine (3.4 mL, 108.1 mmol) in methanol (50 mL) was refluxed overnight. Solvent was removed under reduced pressure. The residue was re-dissolved in ethyl acetate. The solution of ethyl acetate was washed with saturated solution of sodium bicarbonate and brine, dried over sodium carbonate and concentrated to afford 2,4-dimethoxy-benzoic acid hydrazide (3.31g, 78%) as a white solid: ¹H NMR (CDCl₃) δ 8.77 (bs, 1H), 8.15 (d, J=8.8 Hz, 1H), 6.58 (dd, J=8.8, 2.2 Hz, 1H), 6.46 (d, J=2.2 Hz, 1H), 4.10 (bs, 2H), 3.91 (s, 3H), 3.83 (s, 3H).

Ex-58B: A solution of 2,4-dimethoxy-benzoic acid hydrazide (Ex-58A, 11.0 g, 5.1 mmol) and isobutyl-isothiocyanate (0.70g, 6.1 mmol) in ethanol (30 mL) was refluxed for 8 hours. The precipitate was filtered, washed with ethanol, dried in vacuo to afford 1-(2,4-dimethoxy-benzoyl)amino-3-isobutyl-thiourea (1.43g). Additional product (0.1g, 96% overall) was obtained by concentrating the mother liquid. ¹H NMR (CDCl₃) δ 10.71 (bs, 1H), 9.23 (bs, 1H), 8.03 (d, J=8.6 Hz, 1H), 6.98 (bs, 1H), 6.59 (dd, J=8.6, 2.6 Hz, 1H), 6.51 (d, J=2.6 Hz, 1H), 4.02 (s, 3H), 3.86 (s, 3H), 3.41 (dd, J=6.4, 6.6 Hz, 2H), 1.96-1.87 (m, 1H), 0.91 (d, J=6.5 Hz, 6H).

Ex-58C: A solution of 1-(2,4-dimethoxy-benzoyl)amino-3-isobutyl-thiourea (Ex-58B, 0.5g, 1.61 mmol) and sodium hydroxide (0.999M, 4.8 mL, 4.8 mmol) in ethanol (30 mL) was refluxed for one day. The solvent was removed under reduced pressure and the residue re-dissolved in ethyl acetate. The solution of ethyl acetate was washed with water and brine, dried over sodium sulfate, and concentrated to give 5-(2,4-dimethoxy-phenyl)-4-isobutyl-4H-[1,2,4]triazole-3-thiol (0.1 g). Additional product (0.36g, 98% overall) was obtained by extracting the water wash with dichloromethane and a mixture of isopropyl alcohol (33%, v/v, in dichloromethane). ¹H NMR (CDCl₃) δ 10.82 (bs, 1H), 7.24 (d, J=8.1 Hz, 1H), 6.56 (dd, J=8.1, 2.4 Hz, 1H), 6.51 (d, J=2.4 Hz, 1H), 3.85 (s, 3H), 3.77 (s, 3H), 3.72 (d, J=6.7 Hz, 2H), 2.17-2.08 (m, 1H), 0.70 (d, J=6.7 Hz, 6H).

Ex-58D: To a solution of 5-(2,4-dimethoxy-phenyl)-4-isobutyl-4H-[1,2,4]triazole-3-thiol (Ex-58C, 0.1g, 0.34 mmol) in ethanol (10 mL) was added wet Raney Ni (0.27g, 4.6 mmol). The suspension of ethanol was refluxed overnight and then passed through a bed of Hyflo Super Gel and diatomaceous earth. The filtrate was concentrated to afford 3-(2,4-dimethoxy-phenyl)-4-isobutyl-4H-[1,2,4]triazole (0.09g, 100%) as a white solid: ¹H NMR (CDCl₃) δ 8.15 (s, 1H), 7.34 (d, J=7.8 Hz, 1H), 6.57 (dd, J=7.8, 2.3 Hz, 1H), 6.51 (d, J=2.3 Hz, 1H), 3.85 (s, 3H), 3.75 (s, 3H), 3.62 (d, J=7.5 Hz, 2H), 1.89-1.80 (m, 1H), 0.76 (d, J=6.6 Hz, 6H).

Ex-58E: To a solution of 3-(2,4-dimethoxy-phenyl)-4-isobutyl-4H-[1,2,4]triazole (Ex-58D, 0.78g, 2.98 mmol) was added dichloromethyl methyl ether (0.4 mL, 4.48 mmol) followed by addition of titanium tetrachloride (1.0M in dichloromethane, 9.0 mL, 9.0 mmol) over 10 min at 0° C. The reaction mixture was allowed to stir at 0° C. for 30 min and ambient temperature overnight. The reaction mixture was poured into ice. The aqueous solution was extracted with dichloromethane and isopropyl alcohol (33%, v/v, in dichloromethane). The combined dichloromethane and isopropyl alcohol were washed with brine, dried over sodium sulfate and concentrated. The aqueous solution was treated with sodium hydroxide to pH 12 and extracted again with isopropyl alcohol (33%, v/v, in dichloromethane) to give additional product. The crude product was purified by flash chromatography. Elution with methanol (10%, v/v, in dichloromethane) afford 5-(4-isobutyl-4H-[1,2,4]triazol-3-yl)-2,4-dimethoxy-benzaldehyde (0.24g, 28%): ¹H NMR (CDCl₃) δ 10.30 (s, 1H), 8.17 (s, 1H), 7.90 (s, 1H), 6.51 (s, 1H), 4.00 (s, 3H), 3.87 (s, 3H), 3.58 (d, J=7.2 Hz, 2H), 1.91-1.80 (m, 1H), 0.77 (d, J=6.5 Hz, 6H).

Ex-58F: To a solution of 4-acetyl-benzenesulfonamide (Ex-26A, 0.12g, 0.62 mmol) and 5-(4-isobutyl-4H-[1,2,4]triazol-3-yl)-2,4-dimethoxy-benzaldehyde (Ex-58E, 0.18g, 0.62 mmol) in N,N-dimethylformamide (9 mL) was added lithium methoxide (1.0M in methanol, 2.4 mL, 2.4 mmol). The solution was allowed to stir overnight. The reaction was quenched with water. The aqueous solution was washed ethyl acetate, acidified to pH 5, extracted with dichloromethane, isopropyl alcohol (33%, v/v, in dichloromethane). The combined dichloromethane and isopropyl alcohol was washed with brine, dried over sodium sulfate and concentrated. The crude product was then stirred in ethanol (50%, v/v, in acetone) to give the title compound as a light yellow solid: m.p. >240-C. ¹H NMR (DMSO-d₆) δ 8.60 (s, 1H), 8.26 (d, J=8.1 Hz, 2H), 8.06 (d, J=15.3 Hz, 1H), 8.07 (s, 1H), 7.91 (d, J=8.1 Hz, 2H), 7.84 (d, J=15.3 Hz, 1H), 7.50 (s, 1H), 6.84 (s, 1H), 4.01 (s, 3H), 3.87 (s, 3H), 3.61 (d, J=7.3 Hz, 2H), 1.81-1.74 (m, 1H), 0.67 (d, J=16.7 Hz, 6H). MS m/z=471 ([M+H]⁺, 100%).

### Example 59

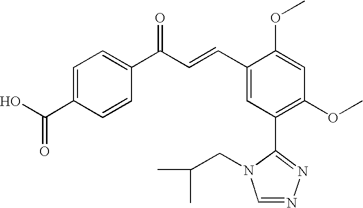

4-{3E-[5-(4-Isobutyl-4H-[1,2,4]triazol-3-yl)-2,4-dimethoxy-phenyl]-acryloyl}-benzoic acid

Ex-59: To a solution of 4-acetyl-benzoic acid (0.12g, 0.75 mmol) and 5-(4-isobutyl-4H-[1,2,4]triazol-3-yl)-2,4-dimethoxy-benzaldehyde (Ex-58E, 0.24g, 0.83 mmol) in N,N-dimethylformamide (6 mL) was added lithium methoxide (11.0M in methanol, 3.0 mL, 3.0 mmol). The solution was allowed to stir overnight and additional lithium methoxide (0.11 g, 2.8 mmol). The reaction was quenched with water after 20 hours. The aqueous solution was washed ethyl acetate, acidified to pH 4. The precipitate was filtered, washed with ethanol and dried in vacuo to afford the title compound as a light yellow solid: m.p. >240° C. (dec.). ¹H NMR (DMSO-d₆) δ 8.59 (s, 1H), 8.18 (d, J=7.9 Hz, 2H), 8.07 (s, 1H), 8.04-8.01 (m, 3H), 7.85 (d, J=15.7 Hz, 1H), 6.84 (s, 1H), 4.06 (s, 3H), 3.92 (s, 3H), 3.66 (d, J=7.2 Hz, 2H), 1.87-1.74 (m, 1H), 0.72 (d, J=6.7 Hz, 6H). MS m/z=436 ([M+H]⁺, 100%).

### Example 60

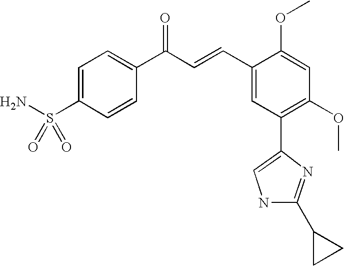

4-{3E-[5-(2-Cyclopropyl-1H-imidazol-4-yl)-2,4-dimethoxy-phenyl]-acryloyl}-benzenesulfonamide

Ex-60: To a solution of 4-acetyl-benzenesulfonamide (Ex-26A, 0.12g, 0.59 mmol) and 5-(2-cyclopropyl-1H-imidazol-4-yl)-2,4-dimethoxy-benzaldehyde (Ex-55B, 0.16 g, 0.59 mmol) in N,N-dimethylformamide (16 mL) was added lithium methoxide (1.0M in methanol, 2.4 mL, 2.4 mmol). The reaction mixture was allowed to stir for 18 hours at ambient temperature. The reaction was quenched with water. The aqueous solution was extracted with dichloromethane. The combined dichloromethane was concentrated. The crude product was purified by flash chromatography. Elution with methanol (10%, v/v, in dichloromethane) gave the title compound as red solid: m.p. 156-160-C. ¹H NMR (DMSO-d₆) δ 11.65 (bs, 1H), 8.32 (s, 1H), 8.19 (d, J=9.0 Hz, 2H), 8.00 (d, J=15.7 Hz, 1H), 7.95 (d, J=9.0 Hz, 2H), 7.62-7.52 (m, 2H), 7.24 (bs, 1H), 6.73 (s, 1H), 3.96 (s, 3H), 3.94 (s, 3H), 1.98-1.94 (m, 1H), 0.88-0.85 (m, 4H). MS m/z=454 ([M+H]⁺, 100%).

### Example 61

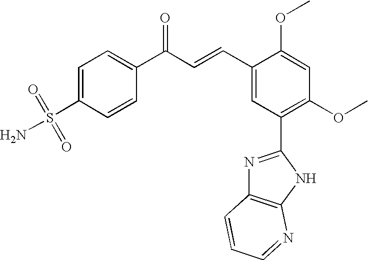

4-{3E-[5-(3H-Imidazo[4,5-b]pyridin-2-yl)-2,4-dimethoxy-phenyl]-acryloyl}-benzenesulfonamide

Ex-61: The title compound was prepared by condensing 5-(3H-imidazo[4,5-b]pyridin-2-yl)-2,4-dimethoxy-benzaldehyde with 4-acetyl-benzenesulfonamide (Ex-26A) in a similar manner as described in Ex-22. Yellow solid, 26% yield, mp>260° C. ¹H-NMR (DMSO-d₆) δ 8.73 (s, 1H), 8.31 (dd, J=1, 4 Hz, 1H), 8.26 (d, J=8 Hz, 2H), 8.05(d, J=16 Hz, 1H), 7.89-7.97 (m, 3H), 7.82(d, J=16 Hz, 1H), 7.17-7.21(m, 1H), 6.89(s, 1H), 4.09 (s, 3H), 4.03 (s, 3H). MS m/z=465([M+H]⁺, 65%), 256 (100%). HRMS (ES+) Calcd. for C₂₃H₂₀N₄O₅S: 465.1232. Found: 465.1240.

### Example 62

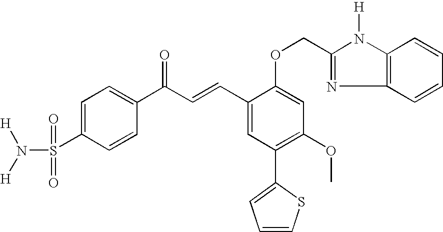

4-{3E-[2-(1H-Benzoimidazol-2-ylmethoxy)-4-methoxy-5-thiophen-2-yl-phenyl]-acryloyl}-benzenesulfonamide

Ex-62A: 2-(1H-Benzoimidazol-2-ylmethoxy)-4-methoxy-5-thiophen-2-yl-benzaldehyde was prepared in a similar manner as described in Ex-1C. Off-white solid, 67% yield, mp 230° C. (dec). ¹H-NMR (300 MHz, DMSO-d₆) δ 10.44 (s, 1H), 8.00 (s, 1H), 7.79-7.84 (m, 2H), 7.49-7.57 (m, 4H), 7.16 (s, 1H), 7.12 (dd, 1H, J=5.4, 3.6 Hz), 5.91 (s, 2H), 4.07 (s, 3H). MS (ESI) m/z=365 ([M+H]⁺, 100%). Anal. Calcd. for C₂₀H₁₇ClN₂O₃S.⅓H₂O: C, 59.04; H, 4.38; N, 6.88; S, 7.88. Found: C, 59.07; H, 4.25; N, 6.85; S, 7.77.

Ex-62B: The title compound was prepared by condensing 2-(1H-benzoimidazol-2-ylmethoxy)-4-methoxy-5-thiophen-2-yl-benzaldehyde (Ex-62A) and 4-acetylbenzenesulfonamide (Ex-26A) in a similar manner as described in Ex-3B. Light orange solid, 56% yield, mp 235-237° C. (dec). ¹H-NMR (300 MHz, DMSO-d₆) δ 8.27 (s, 1H), 8.19 (d, 2H, J=8.4 Hz), 8.11 (d, 1H, J=15.4 Hz), 7.98 (d, 1H, J=15.4 Hz), 7.89 (d, 2H, J=8.4 Hz), 7.66-7.70 (m, 3H), 7.53-7.55 (m, 3H), 7.22-7.27 (m, 2H), 7.12-7.15 (m, 2H), 5.59 (s, 2H), 4.01 (s, 3H). MS (ESI) m/z=546 ([M+H]⁺, 100%). Anal. Calcd. for C₂₈H₂₃N₃O₅S₂: C, 61.64; H, 4.25; N, 7.70; S, 11.75. Found: C, 61.49; H, 4.47; N, 7.74; S, 11.58.

### Example 63

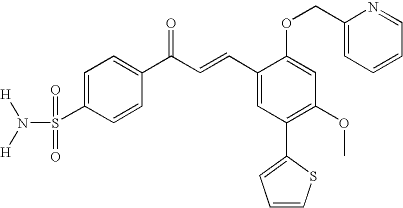

4-{3E-[4-Methoxy-2-(pyridin-2-yl methoxy)-5-thiophen-2-yl-phenyl]-acryloyl}-benzenesulfonamide

Ex-63A: 4-Methoxy-2-(pyridin-2-ylmethoxy)-5-thiophen-2-yl-benzaldehyde was prepared in a similar manner as described in Ex-1C. Yellow solid, 93% yield, mp 93-94° C. ¹H-NMR (300 MHz, CDCl₃) δ 10.49 (s, 1H), 8.62 (d, 1H, J=5.1 Hz), 8.13 (s, 1H), 7.77 (dt, 1H, J=7.5, 1.5 Hz), 7.58 (d, 1H, J=7.5 Hz), 7.44 (dd, 1H, J=3.6, 1.5 Hz), 7.28-7.31 (m, 2H), 7.07 (dd, 1H, J=5.4, 3.6 Hz), 6.64 (s, 1H), 5.39 (s, 2H), 3.94 (s, 3H). MS (ESI) m/z=326 ([M+H]⁺, 100%). Anal. Calcd. for C₁₈H₁₅NO₃S: C, 66.44; H, 4.65; N, 4.30; S, 9.85. Found: C, 66.43; H, 4.72; N, 4.37; S, 9.81.

Ex-63B: The title compound was prepared by condensing 4-methoxy-2-(pyridin-2-ylmethoxy)-5-thiophen-2-yl-benzaldehyde (Ex-63A) and 4-acetylbenzenesulfonamide (Ex-26A) in a similar manner as described in Ex-3B. Yellow solid, 90% yield, mp 188-189° C. ¹H-NMR (300 MHz, DMSO-d₆) δ 8.66 (d, 1H, J=3.6 Hz), 8.28 (s, 1H), 8.21 (d, 2H, J=7.8 Hz), 8.11 (d, 1H, J=15.4 Hz), 7.89-7.99 (m, 4H), 7.57-7.68 (m, 4H), 7.53 (dd, 1H, J=5.4, 1.5 Hz), 7.41-7.45 (m, 1H), 7.13 (dd, 1H, J=5.4, 3.6 Hz), 7.02 (s, 1H), 5.45 (s, 2H), 3.99 (s, 3H). MS (ESI) m/z=507 ([M+H]⁺, 100%). Anal. Calcd. for C₂₆H₂₂N₂O₅S₂.½H₂O: C, 60.57; H, 4.50; N, 5.43; S, 12.44. Found: C, 60.92; H, 4.54; N, 5.48; S, 12.32.

### Example 64

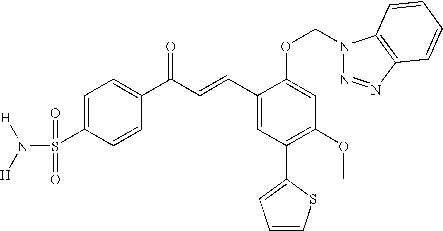

4-{3E-[2-(Benzotriazol-1-ylmethoxy)-4-methoxy-5-thiophen-2-yl-phenyl]-acryloyl}-benzenesulfonamide

Ex-64A: 2-(Benzotriazol-1-ylmethoxy)-4-methoxy-5-thiophen-2-yl-benzaldehyde was prepared in a similar manner as described in Ex-1C. Off-white solid, 92% yield, mp 137-138° C. ¹H-NMR (300 MHz, CDCl₃) δ 10.30 (s, 1H), 8.10 (d, 1H, J=8.1 Hz), 8.06 (s, 1H), 7.75 (d, 1H, J=8.1 Hz), 7.57-7.62 (m, 1H), 7.40-7.48 (m, 2H), 7.30 (d, 1H, J=5.1 Hz), 7.08 (s, 1H), 7.05 (dd, 1H, J=5.1, 3.6 Hz), 6.74 (s, 2H), 4.01 (s, 3H). MS (ESI) m/z=366 ([M+H]⁺, 100%). Anal. Calcd. for C₁₉H₁₅N₃O₃S: C, 62.45; H, 4.14; N, 11.50; S, 8.78. Found: C, 62.69; H, 4.30; N, 11.52; S, 8.62.

Ex-64B: The title compound was prepared by condensing 2-(benzotriazol-1-ylmethoxy)-4-methoxy-5-thiophen-2-yl-benzaldehyde (Ex-64A) and 4-acetylbenzenesulfonamide (Ex-26A) in a similar manner as described in Ex-3B. Light yellow solid, 56% yield, mp 255° C. (dec). ¹H-NMR (300 MHz, DMSO-d₆) δ 8.21 (s, 1H), 8.09 (d, 3H, J=9.4 Hz), 8.01 (d, 1H, J=7.8 Hz), 7.93 (d, 2H, J=7.8 Hz), 7.75 (d, 2H, J=9.4 Hz), 7.56-7.69 (m, 4H), 7.42-7.47 (m, 1H), 7.38 (s, 1H), 7.13 (dd, 1H, J=5.4, 3.6 Hz), 7.05 (s, 2H), 4.05 (s, 3H). MS (ESI) m/z=547 ([M+H]⁺, 100%). Anal. Calcd. C₂₇H₂₂N₄O₅S₂: C, 59.33; H, 4.06; N, 10.25; S, 11.73. Found: C, 59.45; H, 4.27; N, 9.92; S, 11.27.

### Example 65

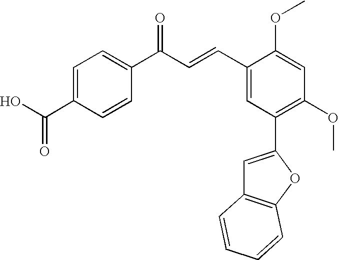

4-[(2E)-3-(5-benzofuran-2-yl-2,4-dimethoxyphenyl)-1-oxo-2-propenyl]-benzoic acid

Ex-65: The title compound was prepared by condensing 5-benzofuran-2-yl-2,4-dimethoxy-benzaldehyde and 4-acetylbenzoic acid in a similar manner as described in Ex-3B. Yellow solid, mp 227-9° C. ¹H-NMR (Acetone-d₆) δ 8.42 (s, 1H), 8.19 (m, 5H), 7.84 (d, J=15.4 Hz, 1H), 7.59 (d, J=8.3 Hz, 1H), 7.53 (d, J=8.3 Hz, 1H), 7.28 (m, 3H), 6.93 (s, 1H), 4.14 (s, 3H), 4.06 (s, 3H).

### Example 66

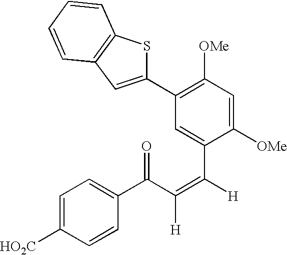

4-[(2Z)-3-(5-benzo[b]thien-2-yl-2,4-dimethoxyphenyl)-1-oxo-2-propenyl]-benzoic acid

4-[(2E)-3-(5-benzo[b]thien-2-yl-2,4-dimethoxyphenyl)-1-oxo-2-propenyl]-benzoic acid from Ex-3 was suspended in MTBE and aged for 12-15 h under blacklight irradiation. The reaction was then concentrated and filtered to remove any remaining starting material. The mother liquor was then evaporated to dryness, the resulting solid was slurried in EtOAc, and filtered affording 4-[(2Z)-3-(5-benzo[b]thien-2-yl-2,4-dimethoxyphenyl)-1-oxo-2-propenyl]-benzoic acid; mp 176-178° C. ¹H NMR (300 MHz, DMSO-d₆) δ 13.2 (s, 1H), 7.99 (s, 4H), 7.87 (m, 2H), 7.76 (dd, 1H, J=7.7, 1.5 Hz), 7.56 (s, 1H), 7.28 (m, 2H), 7.18 (d, 1H, J=12.5 Hz), 6.80 (d, 1H, J=12.5 Hz), 6.71 (s, 1H), 3.95 (s, 3H), 3.77 (s, 3H).

Examples 1, 2, and 4-65 can be isomerized to their Z isomer or to mixtures of their E and Z isomers. This is preferably accomplished by exposure to light.

### Example 67

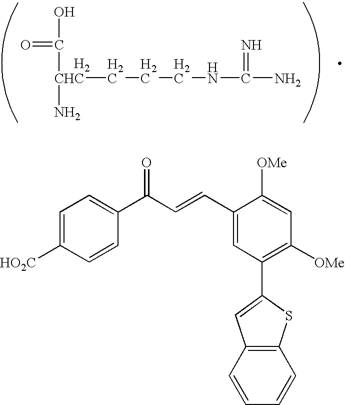

4-[(2E)-3-(5-benzo[b]thien-2-yl-2,4-dimethoxyphenyl)-1-oxo-2-propenyl]-benzoic acid, L-arginine salt

L-Arginine (16.72 g, 96 mmol) and 4-[(2E)-3-(5-benzo[b]thien-2-yl-2,4-dimethoxyphenyl)-1-oxo-2-propenyl]-benzoic acid (42.80 g, 96 mmol) from Ex-3 were dissolved in a 1/1 mixture of water and ethanol (500 mL). The solution was concentrated to dryness under reduced pressure and the resulting oily residue treated with EtOH (500 mL). The mixture was again concentrated to dryness under reduced pressure. The solid residue was triturated for 6 hours in EtOH (500 mL) before the solvent was removed under reduced pressure thus affording the desired product (58 g) as a yellow powder. ¹H NMR (300 MHz, DMSO-d₆) δ 8.39 (s, 1H), 8.12 (m, 3H), 8.03 (m, 2H), 7.99 (m, 3H), 7.95 (d, 2H, J=9.5 Hz), 7.86 (d, 1H, J=7.1 Hz), 7.35 (m, 2H), 6.89 (s, 1H), 4.06 (s, 3H), 4.03 (s, 3H) 3.30 (m, 1H), 3.09 (m, 2H)1.73 (m, 2H), 1.61 (m, 2H).

From the foregoing description, one skilled in the art can easily ascertain the essential characteristics of this invention, and without departing from the spirit and scope thereof, can make various changes and modifications of the invention to adapt it to various usages and conditions.

We claim:

1. A process of manufacturing a chalcone that includes reacting a carbon-linked heteroaryl or heterocyclic substituted benzaldehyde with an acetophenone in a solvent or mixture of solvents in the presence of LiOMe.

2. The process of manufacturing a compound of Formula I or salts thereof of claim 1, 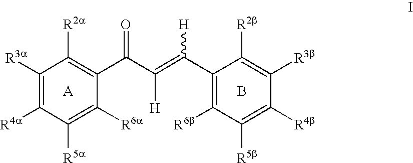
wherein
R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR¹ wherein y is 1, 2, 3, 4, 5, or 6, C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², C(O)NHSO₂R², thiol, —SC(R¹)₂C(O)OH, —SC(R¹)₂C(O)OR², —SCR₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;
wherein at least one of R^{2α}, R^{3α}, R^{4α}, R^{5α}, and R^{6α} must be selected from the group consisting of cyano, tetrazol-5-yl, C(O)OH, C(O)OR², (CH₂)_{y}C(O)OR¹ wherein y is 1, 2, 3, 4, 5, or 6, C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R¹)₂, —C(O)NHSO₂NR⁷R⁸—C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;
R^{2β}, R^{3β}, R^{4β}, R^{5β}, and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, lower alkyl S(O)-lower alkyl, lower alkyl-S(O)₂-lower alkyl, arylsulfinyl lower alkyl, arylsulfonyl lower alkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alditol, carbohydrate, polyol alkyl, alkoxy, lower alkoxy, alkoxy alkoxy alkoxy, —(O(CH₂)₂)₁₋₃-O-lower alkyl, polyoxyalkylene, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, and —(CH₂)_{y}C(O)OH, wherein y is 1, 2, 3, 4, 5, or 6, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂ and —C(O)N(R²)₂;
R⁷ and R⁸ are independently selected from the group consisting of alkyl, alkenyl and aryl and linked together forming a 4- to 12-membered monocyclic, bicylic, tricyclic or benzofused ring;
wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 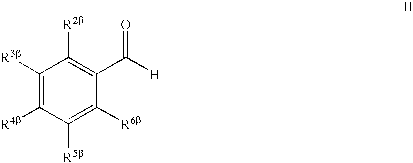
with an acetophenone of Formula III 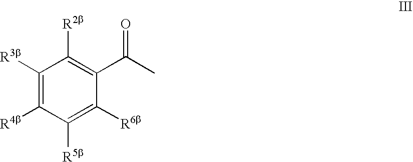
wherein R^{2α}, R^{3α}, R^{4α}, R^{5α}, R^{6α}, R^{2β}, R^{3β}, R^{4β}, R^{5β}, and R^{6β} for Formulas I and III are as defined above;
in a solvent or mixture of solvents in the presence of LiOMe.

3. The process of claim 1, wherein the chalcone is of formula I, 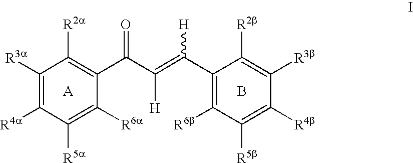
wherein
R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR¹ wherein y is 1, 2, 3, 4, 5, or 6, C(R²)₂C(O)OR¹, —C(O)NH₂, —C(O)NHR, —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²), —C(O)NHSO₂NR⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸;
wherein at least one of R^{2α}, R^{3α}, R^{4α}, R^{5α}, and R^{6α} must be selected from the group consisting of cyano, tetrazol-5-yl, C(O)OH, C(O)OR², (CH₂)_{y}C(O)OR¹ wherein y is 1, 2, 3, 4, 5, or 6, C(R¹)₂C(O)OR¹, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NHSO₂N(R²)₂, —C(O)NHSO₂NR⁷R⁸, —C(O)NHC(O)R², —C(O)NHSO₂R², thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;
R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;
R⁷ and R⁸ are independently selected from the group consisting of alkyl, alkenyl and aryl and linked together forming a 4- to 12-membered monocyclic, bicylic, tricyclic or benzofused ring;
wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 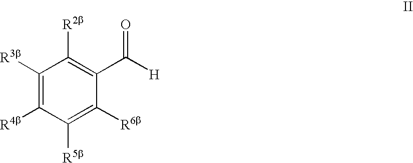
with an acetophenone of Formula III 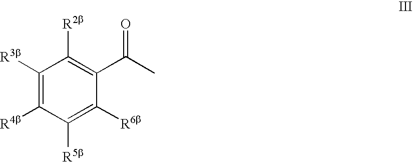
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

4. The process of claim 1, wherein the chalcone is of Formula I or a salt thereof 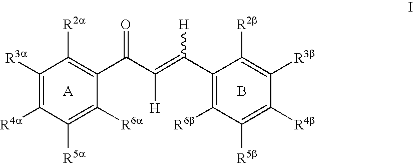
wherein
R^{2α}, R^{3α}, R^{4α}, R^{5α}, and R^{6α} are independently selected from the group consisting of hydrogen, cyano, tetrazol-5-yl, C(O)OH, C(O)OR², (CH₂)_{y}C(O)OR¹ wherein y is 1, 2, 3, 4, 5, or 6, C(R¹)₂C(O)OR¹;
wherein at least one of R^{2α}, R^{3α}, R^{4α}, R^{5α}, and R^{6α} must be selected from the group consisting of cyano, tetrazol-5-yl, C(O)OH, C(O)OR², (CH₂)_{y}C(O)OR¹ wherein y is 1, 2, 3, 4, 5, or 6, C(R¹)₂C(O)OR¹;
R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;
R⁷ and R⁸ are independently selected from the group consisting of alkyl, alkenyl and aryl and linked together forming a 4- to 12-membered monocyclic, bicylic, tricyclic or benzofused ring;
wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 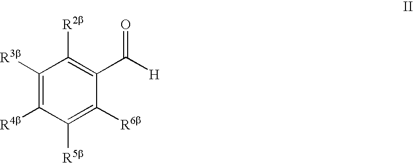
with an acetophenone of Formula III 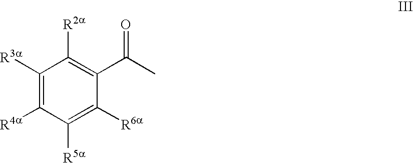
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

5. The process of claim 4 wherein:
R^{2α}, R^{3α}, R^{4α}, R^{5α}, and R^{6α} are independently selected from the group consisting of hydrogen, C(O)OH, C(O)OR², (CH₂)_{y}C(O)OR¹ wherein y is 1, 2, 3, 4, 5, or 6, C(R¹)₂C(O)OR¹;
wherein at least one of R^{2α}, R^{3α}, R^{4α}, R^{5α}, and R^{6α} must be selected from the group consisting of C(O)OH, C(O)OR², (CH₂)_{y}C(O)OR¹ wherein y is 1, 2, 3, 4, 5, or 6, C(R¹)₂C(O)OR¹;
R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, cycloalkyl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, —C(O)NR⁷R⁸, and —C(O)N(R²)₂;
R² is independently selected from the group consisting of alkyl, lower alkyl, cycloalkyl, aryl, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, —C(O)NR⁷R⁸, —C(O)NH₂, and —C(O)N(R²)₂;
R⁷ and R⁸ are independently selected from the group consisting of alkyl, alkenyl and aryl and linked together forming a 5- to 7-membered monocyclic, bicylic, tricyclic or benzofused ring;
wherein R⁷ and R⁸ can be optionally substituted with one or more selected from the group consisting of alkyl, lower alkyl, cycloalkyl, acyl, hydroxy, hydroxyalkyl, heterocyclic, amino, aminoalkyl, —NR⁷R⁸, alkoxy, oxo, cyano, —C(O)NR⁷R⁸, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 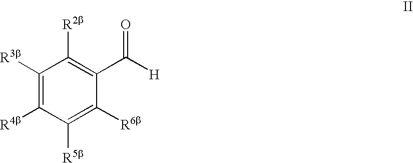
with an acetophenone of Formula III 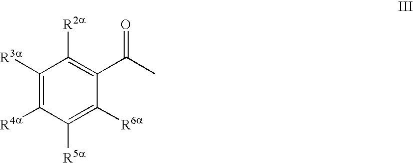
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

6. The process of claim 5 wherein:
R^{2α}, R^{3α}, R^{4α}, R^{5α}, and R^{6α} are independently selected from the group consisting of hydrogen, C(O)OH, and C(O)OR²;
wherein at least one of R^{2α}, R^{3α}, R^{4α}, R^{5α}, and R^{6α} must be selected from the group consisting of C(O)OH and C(O)OR²;
R^{2β}, R^{3β}, R^{4β}, R^{5β}, and R^{6β} are independently selected from the group consisting of hydrogen, halo, alkoxy, alkoxy, heteroaryloxy, heterocyclic, and heteroaryl, all of which can be optionally substituted by one or more selected from the group consisting of halo, alkyl, lower alkyl, cycloalkyl, acyl, hydroxy, hydroxyalkyl, heteroaryl, heterocyclic, alkoxy, oxo, carboxy, carboxyalkyl, alkoxycarbonyl, and —C(O)N(R²)₂;
R² is independently selected from the group consisting of alkyl, lower alkyl, cycloalkyl, arylalkyl, and heteroarylalkyl, wherein all may be substituted by one or more selected from the group consisting of halo, alkyl, lower alkyl, cycloalkyl, acyl, hydroxy, heterocyclic, alkoxy, oxo, —C(O)NH₂, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 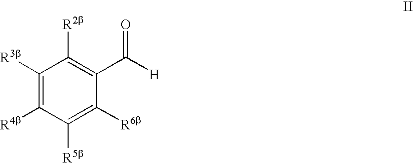
with an acetophenone of Formula III 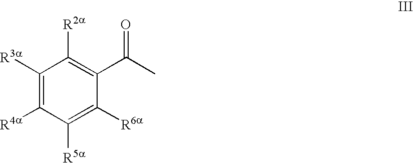
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

7. The process of claim 6 wherein:
R^{2α}, R^{3α}, R^{4α}, R^{5α}, and R^{6α} are independently selected from the group consisting of hydrogen and C(O)OH;
wherein at least one of R^{2α}, R^{3α}, R^{4α}, R^{5α}, and R^{6α} must be C(O)OH;
R^{2β}, R^{3β}, R^{4β}, R^{5β}, and R^{6β} are independently selected from the group consisting of hydrogen, halo, alkoxy, alkoxy, heterocyclic, and heteroaryl, all of which can be optionally substituted by one or more selected from the group consisting of lower alkyl, hydroxy, hydroxyalkyl, heteroaryl, heterocyclic, alkoxy, alkoxycarbonyl, and —C(O)N(R²)₂;
R² is independently selected from the group consisting of alkyl, lower alkyl, arylalkyl, and heteroarylalkyl, wherein all may be substituted by one or more selected from the group consisting of lower alkyl, heterocyclic, alkoxy, —C(O)NH₂, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 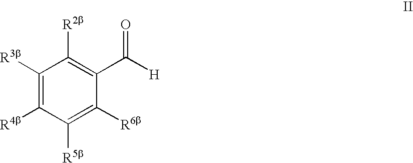
with an acetophenone of Formula III 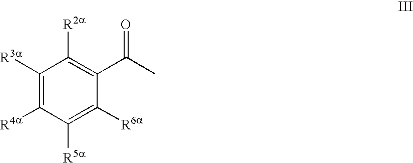
wherein R^{2α}, R^{3α}, R^{4α}R^{5α}, R^{6α}, R^{2β}, R^{3β}, R^{4β}, R^{5β} and R^{6β} for Formula II and III are as defined above;
in a solvent or mixture of solvents in the presence of LiOMe.

8. The process of claim 4 wherein the compound to be manufactured is selected from the group consisting of
4-(3E-{4-Methoxy-2-[2-(2-methoxyethoxy)ethoxy]-5-thiophen-2-yl-phenyl}-acryloyl)-benzoic acid;
4-{3E-[4-(1-Carboxy-1-methyl-ethoxy)-2-methoxy-5-thiophen-2-yl-phenyl]-acryloyl}-benzoic acid;
4-[(2E)-3-(5-Benzo[b]thien-2-yl-2,4-dimethoxyphenyl)-1-oxo-2-propenyl]-benzoic acid;
4-[3E-(2,4-Dimethoxy-5-thiophen-2-yl-phenyl)-acryloyl]-benzoic acid;
4-[3E-(2,6-Dimethoxy-4-thiophen-2-yl-phenyl)-acryloyl]-benzoic acid;
4-{3E-[2,4-Dimethoxy-5-(5-methyl-thiophen-2-yl)-phenyl]-acryloyl}-benzoic acid;
4-[3E-(4-Methoxy-3-thiophen-2-yl-phenyl)-acryloyl]-benzoic acid;
4-[3E-(3-Thiophen-2-yl-phenyl)-acryloyl]-benzoic acid;
3-[3E-(2,4-Dimethoxy-5-thiophen-2-yl-phenyl)-acryloyl]-benzoic acid;
4-[3E-(3-Benzo[b]thiophen-2-yl-2,4-dimethoxy-phenyl)-acryloyl]-benzoic acid;
4-[3E-(2-Methoxy-5-thiophen-2-yl-phenyl)-acryloyl]-benzoic acid;
4-[3E-(2,4-Dimethoxy-5-pyrazin-2-yl-phenyl)-acryloyl]-benzoic acid;
4-(3E-{2-Methoxy-4-[2-(2-methoxy-ethoxy)-ethoxy]-5-thiophen-2-yl-phenyl}-acryloyl)-benzoic acid;
4-{3E-[4-(3-Hydroxy-2-hydroxymethyl-propoxy)-2-methoxy-5-thiophen-2-yl-phenyl]-acryloyl}-benzoic acid;
5-{5-[3-(4-Carboxy-phenyl)-3-oxo-E-propenyl]-2,4-dimethoxy-phenyl}-thiophene-2-carboxylic acid methyl ester;
4-[3E-(4-Ethoxy-2-methoxy-5-thiophen-2-yl-phenyl)-acryloyl]-benzoic acid;
4-[3E-(4-Hydroxy-2-methoxy-5-thiophen-2-yl-phenyl)-acryloyl]-benzoic acid;
4-[3E-(2,4-Dimethoxy-5-thiazol-2-yl-phenyl)-acryloyl]-benzoic acid;
2-{5-[3-(4-Carboxy-phenyl)-3-oxo-E-propenyl]-2,4-dimethoxy-phenyl}-pyrrole-1-carboxylic acid tert-butyl ester;
4-[3E-(2-Hydroxy-4-methoxy-5-thiophen-2-yl-phenyl)-acryloyl]-benzoic acid;
4-{3E-[2-(1-Carboxy-1-methyl-ethoxy)-4-methoxy-5-thiophen-2-yl-phenyl]-acryloyl}-benzoic acid;
4-{3E-[4-Methoxy-2-(2-morpholin-4-yl-ethoxy)-5-thiophen-2-yl-phenyl]-acryloyl}-benzoic acid, hydrochloride;
4-{3E-[5-(1H-Indol-2-yl)-2,4-dimethoxy-phenyl]-acryloyl}-benzoic acid;
4-{3E-[2-(3,5-Dimethyl-isoxazol-4-ylmethoxy)-4-methoxy-5-thiophen-2-yl-phenyl]-acryloyl}-benzoic acid;
4-[3E-(2-Pyrrolidin-1-yl-S-thiophen-2-yl-phenyl)-acryloyl]-benzoic acid;
4-{3E-[2-(3-Hydroxy-2-hydroxymethyl-propoxy)-4-methoxy-5-thiophen-2-yl-phenyl]-acryloyl}-benzoic acid;
4-{3E-[2-(3-Morpholin-4-yl-propoxy)-5-thiophen-2-yl-phenyl]-acryloyl}-benzoic acid, hydrochloride;
4-{3E-[4-Methoxy-2-(3-morpholin-4-yl-propoxy)-5-thiophen-2-yl-phenyl]-acryloyl}-benzoic acid, hydrochloride;
4-[3E-(2-Dimethylcarbamoylmethoxy-4-methoxy-5-thiophen-2-yl-phenyl)-acryloyl]-benzoic acid;
4-[3E-(4-Methoxy-2-{2-[2-(2-methoxy-ethoxy)-ethoxy]-ethoxy}-5-thiophen-2-yl-phenyl)-acryloyl]-benzoic acid;
4-{3E-[2,4-Dimethoxy-5-(2-methyl-thiazol-4-yl)-phenyl]-acryloyl}-benzoic acid;
4-{3E-[5-(1H-Benzoimidazol-2-yl)-2,4-dimethoxy-phenyl]-acryloyl}-benzoic acid;
4-[3E-(2-Carbamoylmethoxy-4-methoxy-5-thiophen-2-yl-phenyl)-acryloyl]-benzoic acid;
4-{3E-[4-Methoxy-2-(2-morpholin-4-yl-2-oxo-ethoxy)-5-thiophen-2-yl-phenyl]-acryloyl}-benzoic acid;
4-(3E-{4-Methoxy-2-[2-(1-methyl-pyrrolidin-2-yl)-ethoxy]-5-thiophen-2-yl-phenyl}-acryloyl)-benzoic acid, hydrochloride;
4-{3E-[2,4-Dimethoxy-5-(1H-pyrazol-4-yl)-phenyl]-acryloyl}-benzoic acid;
4-{3E-[2,4-Dimethoxy-5-(2H-tetrazol-5-yl)-phenyl]-acryloyl}-benzoic acid;
4-{3E-[5-(3H-Imidazo[4,5-b]pyridin-2-yl)-2,4-dimethoxy-phenyl]-acryloyl}-benzoic acid;
4-{3E-[2,4-Dimethoxy-5-(1-methyl-1H-indol-2-yl)-phenyl]-acryloyl}-benzoic acid;
4-[(2E)-3-(5-Benzofuran-2-yl-2,4-dimethoxyphenyl)-1-oxo-2-propenyl]-benzoic acid;
4-{3E-[5-(2-Cyclopropyl-1H-imidazol-4-yl)-2,4-dimethoxy-phenyl]-acryloyl}-benzoic acid, hydrochloride; and
4-{3E-[5-(4-Isobutyl-4H-[1,2,4]triazol-3-yl)-2,4-dimethoxy-phenyl]-acryloyl}-benzoic acid.

9. The process of claim 1, wherein the chalcone is of Formula I or a salt thereof 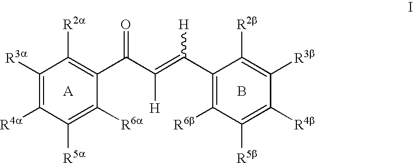
wherein
R^{2α}, R^{3α}, R^{4α}, R^{5α}, and R^{6α} are independently selected from the group consisting of hydrogen, thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR²—SO₂NHC(O)NHR², —SO₂NHC(O)N(R²)₂—SO₂NHC(O)NR⁷R⁸;
wherein at least one of R^{2α}, R^{3α}, R^{4α}, R^{5α}, and R^{6α} must be selected from the group consisting of thiol, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, and —SO₂NHC(O)NR⁷R⁸;
R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, cycloalkyl, aryl, heteroaryl, heterocyclic, arylalkyl, heteroarylalkyl, and heterocyclicalkyl, wherein all may be optionally substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;
R⁷ and R⁸ are independently selected from the group consisting of alkyl, alkenyl and aryl and linked together forming a 4- to 12-membered monocyclic, bicylic, tricyclic or benzofused ring;
wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 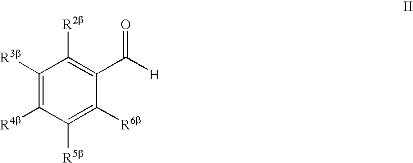
with an acetophenone of Formula III 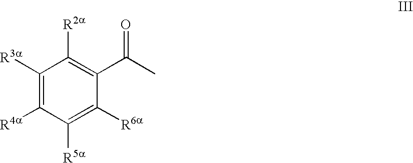
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

10. The process of claim 9 wherein
R^{2α}, R^{3α}, R^{4α}, R^{5α}, and R^{6α} are independently selected from the group consisting of hydrogen, —SO₂NH₂, —SO₂NHR², —SO₂N(R²)₂, SO₂NR⁷R⁸, —SO₂NHC(O)R², —SO₂NHC(O)NHR², —SO₂NHC(O)N(R²)₂, —SO₂NHC(O)NR⁷R⁸;
wherein at least one of R^{2α}, R^{3α}, R^{4α}, R^{5α}, and R^{6α} must be selected from the group consisting of —SO₂NH₂, —SO₂NHR², —SO₂N(R²)₂, SO₂NR⁷R⁸, —SO₂NHC(O)R², —SO₂NHC(O)NHR², —SO₂NHC(O)N(R²)₂, and —SO₂NHC(O)NR⁷R⁸;
R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, cycloalkyl, aryl, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, —C(O)NR⁷R⁸, —C(O)NH₂ and —C(O)N(R²)₂;
R⁷ and R⁸ are independently selected from the group consisting of alkyl, alkenyl and aryl and linked together forming a 5- to 7-membered monocyclic, bicylic, tricyclic or benzofused ring;
wherein R⁷ and R⁸ can be optionally substituted with one or more selected from the group consisting of alkyl, lower alkyl, cycloalkyl, acyl, hydroxy, hydroxyalkyl, heterocyclic, amino, aminoalkyl, —NR⁷R⁸, alkoxy, oxo, cyano, —C(O)NR⁷R⁸, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 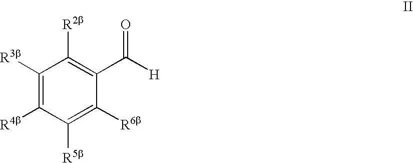
with an acetophenone of Formula III 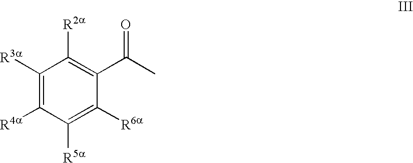
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

11. The process claim 10 wherein
R^{2α}, R^{3α}, R^{4α}, R^{5α}, and R^{6α} are independently selected from the group consisting of hydrogen, —SO₂NH₂, —SO₂NHR², —SO₂N(R²)₂, and SO₂NR⁷R⁸;
wherein at least one of R^{2α}, R^{3α}, R^{4α}, R^{5α}, and R^{6α} must be selected from the group consisting of —SO₂NH₂, —SO₂NHR², —SO₂N(R²)₂, SO₂NR⁷R⁸, and —SO₂NHC(O)R²;
R^{2β}, R^{3β}, R^{4β}, R^{5β}, and R^{6β} are independently selected from the group consisting of hydrogen, halo, alkoxy, heteroaryloxy, heterocyclic, and heteroaryl, all of which can be optionally substituted by one or more selected from the group consisting of halo, alkyl, lower alkyl, cycloalkyl, acyl, hydroxy, hydroxyalkyl, heteroaryl, heterocyclic, alkoxy, oxo, cyano, carboxy, carboxyalkyl, alkoxycarbonyl, and —C(O)N(R²)₂;
R² is independently selected from the group consisting of alkyl, lower alkyl, cycloalkyl, arylalkyl, and heteroarylalkyl wherein all may be substituted by one or more selected from the group consisting of halo, alkyl, lower alkyl, cycloalkyl, acyl, hydroxy, heterocyclic, alkoxy, oxo, —C(O)NH₂, and —C(O)N(R²)₂;
R⁷ and R⁸ are independently selected from the group consisting of alkyl, alkenyl and aryl and linked together forming a 5- to 7-membered monocyclic, bicylic, tricyclic or benzofused ring;
wherein R⁷ and R⁸ can be optionally substituted with one or more selected from the group consisting of lower alkyl, hydroxy, hydroxyalkyl, heterocyclic, amino, aminoalkyl, and cyano;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 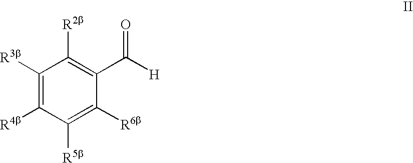
with an acetophenone of Formula III 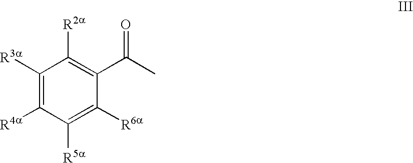
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

12. The process of claim 11 wherein
R^{2α}, R^{3α}, R^{4α}, R^{5α}, and R^{6α} are independently selected from the group consisting of hydrogen and —SO₂NH₂;
wherein at least one of R^{2α}, R^{3α}, R^{4α}, R^{5α}, and R^{6α} must be —SO₂NH₂;
R^{2β}, R^{3β}, R^{4β}, R^{5β}, and R^{6β} are independently selected from the group consisting of hydrogen, halo, alkoxy, heterocyclic, and heteroaryl, all of which can be optionally substituted by one or more selected from the group consisting of lower alkyl, hydroxy, hydroxyalkyl, heteroaryl, heterocyclic, alkoxy, cyano, alkoxycarbonyl, and —C(O)N(R²)₂;
R² is independently selected from the group consisting of alkyl, lower alkyl, arylalkyl, and heteroarylalkyl wherein all may be substituted by one or more selected from the group consisting of lower alkyl, heterocyclic, alkoxy, —C(O)NH₂, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 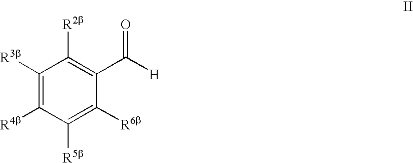
with an acetophenone of Formula III 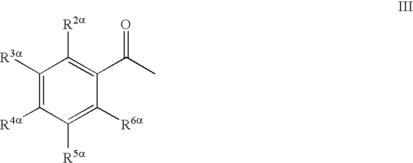
wherein R^{2α}, R^{3α}, R^{4α}, R^{5α}, R^{6α}, R^{2β}, R^{3β}, R^{4β}, R^{5β} and R^{6β} for Formula II and III are as defined above;
in a solvent or mixture of solvents in the presence of LiOMe.

13. The process of claim 9 wherein the compound is selected from the group consisting of:
4-[3E-(5-Benzo[b]thiophen-2-yl-2,4-dimethoxy-phenyl)-acryloyl]-benzenesulfonamide;
4-{3E-[4-Methoxy-2-(2-morpholin-4-yl-ethoxy)-5-thiophen-2-yl-phenyl]-acryloyl}-benzenesulfonamide;
2-{5-Methoxy-2-[3-oxo-3-(4-aminosulfonyl-phenyl)-E-propenyl]-4-thiophen-2-yl-phenoxy}-2-methyl-propionic acid;
2-{2,4-Dimethoxy-5-[3-oxo-3-(4-aminosulfonyl-phenyl)-E-propenyl]-phenyl}-indole-1-carboxylic acid tert-butyl ester;
4-{3E-[5-(1H-Indol-2-yl)-2,4-dimethoxy-phenyl]-acryloyl}-benzenesulfonamide;
4-{3E-[4-Methoxy-2-(3-morpholin-4-yl-propoxy)-5-thiophen-2-yl-phenyl]-acryloyl}-benzenesulfonamide;
4-{3E-[2-(3-Hydroxy-2-hydroxymethyl-propoxy)-4-methoxy-5-thiophen-2-yl-phenyl]-acryloyl}-benzenesulfonamide;
4-{3E-[4-Methoxy-2-(1H-tetrazol-5-ylmethoxy)-5-thiophen-2-yl-phenyl]-acryloyl}-benzenesulfonamide;
4-{3E-[2,4-Dimethoxy-5-(1-methyl-1H-indol-2-yl)-phenyl]-acryloyl}-benzoic acid;
4-{3-[3E-(2,3-Dihydro-furan-2-yl)-phenyl]-acryloyl}-benzenesulfonamide;
4-{3E-[5-(2,5-Dihydro-furan-2-yl)-2,4-dimethoxy-phenyl]-acryloyl}-benzenesulfonamide;
4-{3E-[4-Methoxy-2-(6-methyl-pyridin-2-yloxy)-5-thiophen-2-yl-phenyl]-acryloyl}-benzenesulfonamide;
4-[3E-(2,4-Dimethoxy-5-pyridin-3-yl-phenyl)-acryloyl]-benzenesulfonamide;
4-{3E-[5-(2-Cyclopropyl-1H-imidazol-4-yl)-2,4-dimethoxy-phenyl]-acryloyl}-benzoic acid, hydrochloride;
4-{3E-[4-(3-Hydroxy-2-hydroxymethyl-propoxy)-2-methoxy-5-thiophen-2-yl-phenyl]-acryloyl}-benzenesulfonamide;
4-{3E-[2,4-Dimethoxy-5-(1-methyl-1H-indol-2-yl)-phenyl]-acryloyl}-benzenesulfonamide;
4-{3E-[5-(4-Isobutyl-4H-[1,2,4]triazol-3-yl)-2,4-dimethoxy-phenyl]-acryloyl}-benzenesulfonamide;
4-{3E-[5-(4-Isobutyl-4H-[1,2,4]triazol-3-yl)-2,4-dimethoxy-phenyl]-acryloyl}-benzoic acid;
4-{3E-[5-(2-Cyclopropyl-1H-imidazol-4-yl)-2,4-dimethoxy-phenyl]-acryloyl}-benzenesulfonamide;
4-{3E-[5-(3H-Imidazo[4,5-b]pyridin-2-yl)-2,4-dimethoxy-phenyl]-acryloyl}-benzenesulfonamide;
4-{3E-[2-(1H-Benzoimidazol-2-ylmethoxy)-4-methoxy-5-thiophen-2-yl-phenyl]-acryloyl}-benzenesulfonamide;
4-{3E-[4-Methoxy-2-(pyridin-2-ylmethoxy)-5-thiophen-2-yl-phenyl]-acryloyl}-benzenesulfonamide; and
4-{3E-[2-(Benzotriazol-1-ylmethoxy)-4-methoxy-5-thiophen-2-yl-phenyl]-acryloyl}-benzenesulfonamide.

14. The process of claim 1, wherein the chalcone is of Formula I or a salt thereof 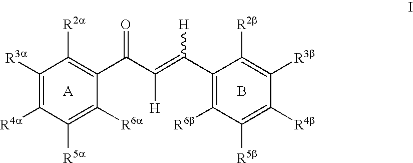
wherein
R^{2α}, R^{3α}, R^{4α}, R^{5α}, and R^{6α} are independently selected from the group consisting of hydrogen, —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NHSO₂NHR², —C(O)NH SO₂N(R²), —C(O)NHSO₂NR⁷R⁸, —C(O)NHC(O)R², —C(O)NHSO₂R²;
wherein at least one of R^{2α}, R^{3α}, R^{4α}, R^{5α}, and R^{6α} must be selected from the group consisting of —C(O)NH₂, —C(O)NHR², —C(O)N(R²)₂, —C(O)NR⁷R⁸, —C(O)NHC(O)NHR², —C(O)NHC(O)N(R²)₂, —C(O)NHC(O)NR⁷R⁸, —C(O)NRSO₂NHR², —C(O)NHSO₂N(R²)₂, —C(O)NHSO₂NR⁷R⁸, —C(O)NHC(O)R², —C(O)NHSO₂R²;
R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, cycloalkyl, aryl, heteroaryl, heterocyclic, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, —C(O)NH₂, and —C(O)N(R²)₂;
R⁷ and R⁸ are independently selected from the group consisting of alkyl, alkenyl and aryl and linked together forming a 4- to 12-membered monocyclic, bicylic, tricyclic or benzofused ring;
wherein R⁷ and R⁸ can be optionally substituted with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 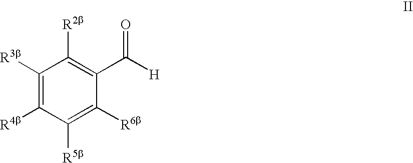
with an acetophenone of Formula III 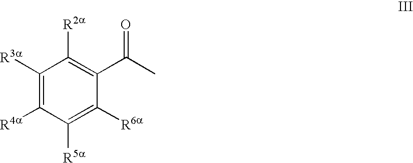
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

15. The process of claim 14 wherein
R^{2α}, R^{3α}, R^{4α}, R^{5α}, and R^{6α} are independently selected from the group consisting of hydrogen, —C(O)NH₂, —C(O)NHR², —C(O)N(R²)₂, —C(O)NR⁷R⁸, —C(O)NHSO₂NR⁷R⁸, —C(O)NHC(O)R², —C(O)NHSO₂R²;
wherein at least one of R^{2α}, R^{3α}, R^{4α}, R^{5α}, and R^{6α} must be selected from the group consisting of —C(O)NH₂, —C(O)NHR², —C(O)N(R²)₂, —C(O)NR⁷R⁸, —C(O)NHSO₂NR⁷R⁸, —C(O)NHC(O)R², —C(O)NHSO₂R²;
R^{2β}, R^{3β}, R^{4β}, R^{5β}, and R^{6β} are independently selected from the group consisting of hydrogen, halo, alkoxy, alkoxy alkoxy alkoxy, amino, NR⁷R⁸, heteroaryloxy, heterocyclic, and heteroaryl, all of which can be optionally substituted by one or more selected from the group consisting of halo, alkyl, lower alkyl, alkenyl, cycloalkyl, acyl, hydroxy, hydroxyalkyl, heteroaryl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, cycloalkyl, aryl, arylalkyl, heteroarylalkyl, and heterocyclicalkyl, wherein all may be substituted by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, —C(O)NR⁷R⁸, —C(O)NH₂ and —C(O)N(R²)₂;
R⁷ and R⁸ are independently selected from the group consisting of alkyl, alkenyl and aryl and linked together forming a 5- to 7-membered monocyclic benzofused ring;
wherein R⁷ and R⁸ can be optionally substituted with one or more selected from the group consisting of alkyl, lower alkyl, cycloalkyl, acyl, hydroxy, hydroxyalkyl, heterocyclic, amino, aminoalkyl, alkoxy, cyano, —C(O)NR⁷R⁸, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 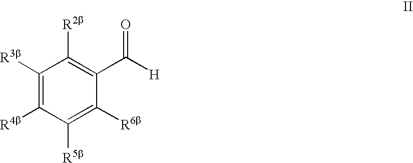
with an acetophenone of Formula III 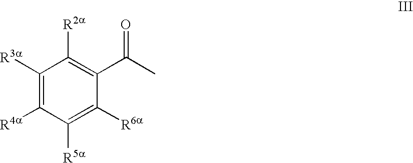
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

16. The process of claim 15 wherein
R^{2α}, R^{3α}, R^{4α}, R^{5α}, and R^{6α} are independently selected from the group consisting of hydrogen, —C(O)NH₂, —C(O)NHR², —C(O)NHC(O)R², —C(O)NHSO₂R²;
wherein at least one of R^{2α}, R^{3α}, R^{4α}, R^{5α}, and R^{6α} must be selected from the group consisting of —C(O)NH₂, —C(O)NHR², —C(O)NHC(O)R², —C(O)NHSO₂R²;
R^{2β}, R^{3β}, R^{4β}, R^{5β}, and R^{6β} are independently selected from the group consisting of hydrogen, halo, alkoxy, heteroaryloxy, heterocyclic, and heteroaryl, all of which can be optionally substituted by one or more selected from the group consisting of halo, alkyl, lower alkyl, cycloalkyl, acyl, hydroxy, hydroxyalkyl, heteroaryl, heterocyclic, alkoxy, oxo, carboxy, carboxyalkyl, alkoxycarbonyl, and —C(O)N(R²)₂;
R² is independently selected from the group consisting of alkyl, lower alkyl, cycloalkyl, arylalkyl, and heteroarylalkyl, wherein all may be substituted by one or more selected from the group consisting of halo, alkyl, lower alkyl, cycloalkyl, acyl, hydroxy, heterocyclic, alkoxy, oxo, —C(O)NH₂, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 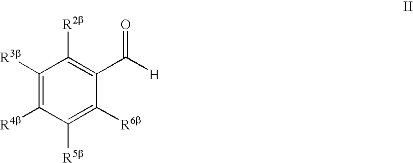
with an acetophenone of Formula III 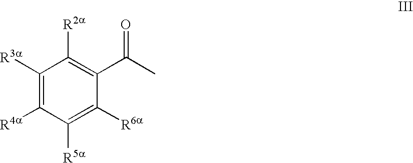
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

17. The process of claim 16 wherein
R^{2α}, R^{3α}, R^{4α}, R^{5α}, and R^{6α} are independently selected from the group consisting of hydrogen, —C(O)NH₂, —C(O)NHR², —C(O)NHC(O)R², —C(O)NHSO₂R²;
wherein at least one of R^{2α}, R^{3α}, R^{4α}, R^{5α}, and R^{6α} must be selected from the group consisting of —C(O)NH₂, —C(O)NHR², —C(O)NHC(O)R², —C(O)NHSO₂R²;
R^{2β}, R^{3β}, R^{4β}, R^{5β}, and R^{6β} are independently selected from the group consisting of hydrogen, halo, alkoxy, heterocyclic, and heteroaryl, all of which can be optionally substituted by one or more selected from the group consisting of lower alkyl, hydroxy, hydroxyalkyl, heteroaryl, heterocyclic, alkoxy, oxo, alkoxycarbonyl, and —C(O)N(R²)₂;
R² is independently selected from the group consisting of lower alkyl, arylalkyl, and heteroarylalkyl, wherein all may be substituted by one or more selected from the group consisting of lower alkyl, heterocyclic, alkoxy, —C(O)NH₂, and —C(O)N(R²)₂;
wherein at least one of R^{2β}, R^{3β}, R^{4β}, R^{5β}, and R^{6β} must be an optionally substituted carbon-carbon linked heterocyclic or heteroaryl;
comprising:
reacting a carbon-linked heteroaryl or heterocyclic substituted benzaldehyde of Formula II 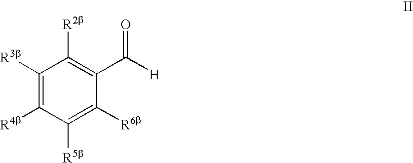
with an acetophenone of Formula III 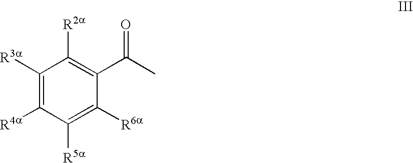
wherein R^{2α}, R^{3α}, R^{4α}, R^{5α}, R^{6α}, R^{2β}, R^{3β}, R^{4β}, R^{5β}, and R^{6β} for Formula II and III are as defined above;
in a solvent or mixture of solvents in the presence of LiOMe.

18. The process of claim 14 wherein the compound is selected from the group consisting of
4-{3E-[4-Methoxy-2-(2-morpholin-4-y 3-ethoxy)-5-thiophen-2-y3-phenyl]-acryloyl}-benzamide;
4-[3E-(5-Benzo[b]thiophen-2-yl-2,4-dimethoxy-phenyl)-acryloyl]-benzamide; and
4-{3E-[4-Methoxy-2-(3-morpholin-4-yl-propoxy)-5-thiophen-2-yl-phenyl]-acryloyl}-benzamide.

19. A process of manufacturing 4-[(2E)-3-(5-benzo[b]thien-2-yl-2,4-dimethoxyphenyl)-1-oxo-2-propenyl]-benzoic acid, 4-[(2Z)-3-(5-benzo[b]thien-2-yl-2,4-dimethoxyphenyl)-1-oxo-2-propenyl]-benzoic acid, or mixtures thereof comprising:
reacting the compound of Formula IV 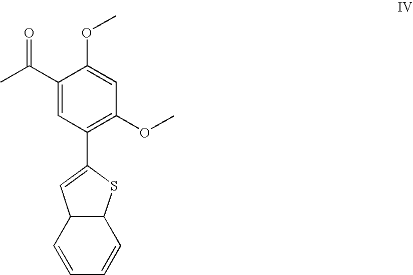
with the compound of Formula V 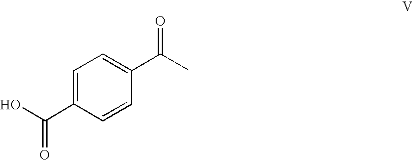
in a solvent or mixture of solvents in the presence of LiOMe.

20. The process of claim 19 wherein said method is the method of manufacturing 4-[(2E)-3-(5-benzo[b]thien-2-yl-2,4-dimethoxyphenyl)-1-oxo-2-propenyl]-benzoic acid.

21. The process of claim 19 further comprising:
isolating 4-[(2E)-3-(5-benzo[b]thien-2-yl-2,4-dimethoxyphenyl)-1-oxo-2-propenyl]-benzoic acid;
isomerizing said 4-[(2E)-3-(5-benzo[b]thien-2-yl-2,4-dimethoxyphenyl)-1-oxo-2-propenyl]-benzoic acid to form 4-[(2Z)-3-(5-benzo[b]thien-2-yl-2,4-dimethoxyphenyl)-1-oxo-2-propenyl]-benzoic acid.

22. A process of manufacturing 4-[(2E)-3-(5-benzofuran-2-yl-2,4-dimethoxyphenyl)-1-oxo-2-propenyl]-benzoic acid, 4-[(2Z)-3-(5-benzofuran-2-yl-2,4-dimethoxyphenyl)-1-oxo-2-propenyl]-benzoic acid, or mixtures thereof comprising:
reacting the compound of Formula VI 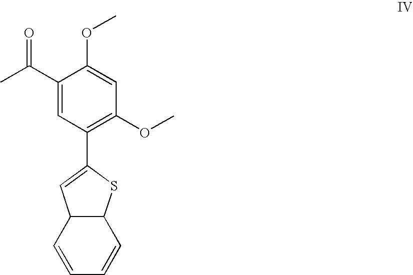
with the compound of Formula V 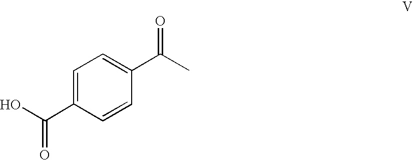
in a solvent or mixture of solvents in the presence of LiOMe.

23. The process of claim 22 wherein said method is the method of manufacturing 4-[(2E)-3-(5-benzofuran-2-yl-2,4-dimethoxyphenyl)-1-oxo-2-propenyl]-benzoic acid.

24. The process of claim 21 further comprising:
isolating 4-[(2E)-3-(5-benzofuran-2-yl-2,4-dimethoxyphenyl)-1-oxo-2-propenyl]-benzoic acid;
isomerizing said 4-[(2E)-3-(5-benzofuran-2-yl-2,4-dimethoxyphenyl)-1-oxo-2-propenyl]-benzoic acid to form 4-[(2Z)-3-(5-benzofuran-2-yl-2,4-dimethoxyphenyl)-1-oxo-2-propenyl]-benzoic acid.

25. A compound selected from the group consisting of 4-[(2E)-3-(5-benzo[b]thien-2-yl-2,4-dimethoxyphenyl)-1-oxo-2-propenyl]-benzoic acid L-arginine salt, 4-[(2Z)-3-(5-benzo[b]thien-2-yl-2,4-dimethoxyphenyl)-1-oxo-2-propenyl]-benzoic acid L-arginine salt, and mixtures thereof.

26. A compound of the formula

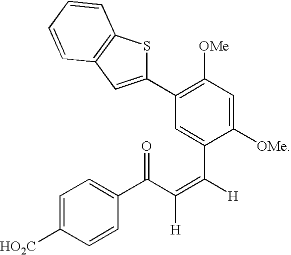

27. A pharmaceutical composition comprising a therapeutically effective amount of a compound selected from the group consisting of 4-[(2E)-3-(5-benzo[b]thien-2-yl-2,4-dimethoxyphenyl)-1-oxo-2-propenyl]-benzoic acid L-arginine salt, 4-[(2Z)-3-(5-benzo[b]thien-2-yl-2,4-dimethoxyphenyl)-1-oxo-2-propenyl]-benzoic acid L-arginine salt, and mixtures thereof together with one or more pharmaceutically acceptable diluent or carrier.

28. A pharmaceutical composition comprising a therapeutically effective amount of 4-[(2Z)-3-(5-benzo[b]thien-2-yl-2,4-dimethoxyphenyl)-1-oxo-2-propenyl]-benzoic acid together with one or more pharmaceutically acceptable diluent or carrier.

29. A compound selected from the group consisting of 4-[(2E)-3-(5-benzofuran-2-yl-2,4-dimethoxyphenyl)-1-oxo-2-propenyl]-benzoic acid, 4-[(2Z)-3-(5-benzofuran-2-yl-2,4-dimethoxyphenyl)-1-oxo-2-propenyl]-benzoic acid, and mixtures thereof.

30. A pharmaceutical composition comprising a therapeutically effective amount of a compound selected from the group consisting of 4-[(2E)-3-(5-benzofuran-2-yl-2,4-dimethoxyphenyl)-1-oxo-2-propenyl]-benzoic acid, 4-[(2Z)-3-(5-benzofuran-2-yl-2,4-dimethoxyphenyl)-1-oxo-2-propenyl]-benzoic acid, and mixtures thereof together with one or more pharmaceutically acceptable diluent or carrier.

31. A method for the treatment or prophylaxis of an inflammatory disorder, comprising administering an effective amount of a compound of claim 24, 25 or 28.

32. The method of claim 31, wherein the disorder is arthritis.

33. The method of claim 31, wherein the disorder is rheumatoid arthritis.

34. The method of claim 31, wherein the disorder is asthma.

35. The method of claim 31, wherein the treatment is disease modifying for the treatment of rheumatoid arthritis.

36. The method of claim 31, wherein the disorder is allergic rhinitis.

37. The method of claim 31, wherein the disorder is chronic obstructive pulmonary disease.

38. The method of claim 31, wherein the disorder is atherosclerosis.

39. The method of claim 31, wherein the disorder is restinosis.
